(19) United States
(12) Patent Application Publication
(10) Pub. No.: US 2010/0010357 A1
(43) Pub. Date: Jan. 14, 2010

(54) Disposable air bag for a blood pressure measuring device and a method of making the same

(76) Inventor: Morris Ostrowiecki, Tokyo (JP)

Correspondence Address: MORRIS OSTROWIECKI, TSUSHIMA BLD. 4F 5-10-20 MINAMI AOYAMA, MINATO-KU, TOKYO, 107-0062, JP

(21) Appl. No.: 12/216,661

(22) Filed: Jul. 9, 2008

(51) Int. Cl. A61B 5/022

(52) U.S. Cl. 600/499

(57) ABSTRACT

An disposable air bag for measuring blood pressure having a diaphragm and a nipple mounted therein, the diaphragm having a plurality of waves for allowing the diaphragm to expand easily out of the air bag and a clip for disconnecting and connecting the nipple to and from one end of an air hose the other end of which is connected to an electronic blood pressure measuring device.

Also disclosed is a blood pressure measuring device having ABS storing means for storing the stretching characteristics of an air bag as a function of the air volume in the air bag, AABPOA calculating means for calculating the actual pressure on the artery, and systolic/diastolic algorithm calculating means for determining the systolic and diastolic blood pressures partially based on the AABPOA calculating means.

### BACKGROUND OF THE INVENTION

Presently, when measuring the blood pressure of patients in hospitals, a nurse goes from patient to patient and applies an armband that is inflated and then used in conjunction with an electronic device to measure and display the blood pressure. The armband and the electronic device are permanently connected to each other by a flexible air hose. However, since the armband is not sterilized when transferring the armband from patient to patient, it may lead to viruses being passed from one patient to another and may result in death of some patients as a consequence. Further, this method requires much time to apply and remove the armband each time by the nurse as well as creates a lot of discomfort to the patient. Furthermore, the process of applying a large arm band may cause some patients to become aggravated, agitated, frightened, resulting in the patients blood pressure going up and, accordingly, providing the wrong information about the patients actual physical condition. Furthermore, many times the patient may be asleep when the nurse comes around to check his or her blood pressure and accordingly, will have to wake up the patient, which is very undesirable. Furthermore, the nurse must spend a lot of time applying and removing the arm bandage from the patients arm, time she could use to do other important things for patients.

### SUMMARY OF THE INVENTION

A major object of the present invention is to overcome the drawbacks mentioned above.

Another object of the present invention is to provide a disposable air bag for measuring blood pressure according to the present invention;

Another object of the present invention is to provide a disposable air bag which doubles up as a patient identification band;

Another object of the present invention is to provide an air bag connecting means for connectively-disconnecting an air bag from one end of an air hose, the other end of the hose being connected to an electronic blood pressure measuring device;

Another object of the present invention is to provide an air bag connecting means which is simple in structure and easy to hermetically connect and disconnect from the air bag:

Another object of the present invention is to provide an air bag connecting means which is made of only one part;

Another object of the present invention is to provide an air bag connecting means which does not disturb or wake up the patient when connected or disconnected to an air bag on the wrist of the patient:

Another object of the present invention is to provide an air bag connecting means which is in the form of a clip, the clip being made of one piece of resilient plastic:

Another object of the present invention is to provide an air bag connecting means which is in the form of a clip having an upper surface thereof in the shape of a cartoon figure, such as snoopy, mickey mouse a frog or any other friendly looking character, which will cause the patient to relax rather then tense up when their blood pressure is about to be measured.

Another object of the present invention is to provide a disposable air bag having patient identification means for identifying the patient the air bag is attached to;

Another object of the present invention is to provide a disposable air bag having an air valve formed therewith, said air valve having air hose attaching means for attaching an air hose thereto so as to enable the pressurization of said air bag through said air valve. Another object of the present invention is to provide a disposable air bag having at least two layers of air bags, so that no matter how tight or loose the patient mounts the air bag, the correct systolic and diastolic blood pressure measurements can be achieved.

Another object of the present invention is to provide a disposable air bag which is light, cheap, simple and is easy to manufacture:

Another object of the present invention is to provide an air bag for measuring blood pressure, the air bag comprising two or more different materials each of which exhibit different desired characteristics for facilitating the measurement of blood pressure.

Another object of the present invention is to provide an air bag for measuring blood pressure, the air bag having no protruding parts on either the inner or outer surface thereof, so that it is very comfortable for the user to wear.

Another object of the present invention is to provide an air bag for measuring blood pressure having a strap around an air bag, the strap being bendable but not stretchable, whereby, when the air bag is inflated, the air bag expands inwardly in a radial direction only, so that no stiff or hard cover is required to be placed around the outer surface thereof.

Another object of the present invention is to provide an air bag for measuring blood pressure, the air bag comprising one layer comprising one material which is easily bendable having a plurality of protrusions formed along the surface thereof, so that certain protrusions in said protrusions positioned over the radial artery can press towards the radial artery with minimal effort according to the present invention.

Another objective of the present invention is to provide an electronic blood pressure measuring device having;

an air bag stretching characteristics table stored therein for storing the air pressure in the air bag as a function of the air volume in the air bag (hereinafter referred to as ABAV-ABAP stretching characteristics or ABS characteristics); and means for calculating the actual air pressure the air in the air bag exerts on the radial artery (hereinafter referred to as AAPOA), so that regardless of how tight or lose the user of the air bag mounts the air bag around his or her wrist, the correct systolic and diastolic pressures can be obtained.

Another object of the present invention is to provide a diaphragm for an air bag which is very thin at a central portion thereof and gradually increases in thickness outwardly from the central portion of the diaphragm, so that when the diaphragm is inflated, the thinnest part of the diaphragm presses against the radial artery first, and accordingly, the blood pressure (hereinafter referred to as BP) as well as changes in blood pressure due to blood pulses (hereinafter referred to as BPP) inside the radial artery (hereafter collectively referred to as blood pressure signature or BPS) are faithfully converted to corresponding air pressure (hereinafter referred to as AP) and air pressure pulses (hereinafter referred to as APP) inside the air bag in which the diaphragm is mounted in (hereinafter collectively referred to as air pressure signature or APS), whereby, blood pressure signature BPS is very faithfully converted (i.e. transformed) to air pressure signature APS.

Another object of the present invention is to provide a diaphragm for an air bag which is very thin at a central portion thereof and gradually increases in thickness outwardly from the central portion of the diaphragm, so that when the diaphragm is inflated, the thinnest part of the diaphragm presses against the radial artery first and the outer part of the diaphragm BLOCKS the central portion of the diaphragm from moving (i.e. escaping) in the lateral direction (hereinafter referred to as diaphragm lateral escape prevention means or DLEPM) and only allows the central portion to move in the radial direction towards the radial artery.

Another object of the present invention is to provide an air bag for measuring blood pressure, the air bag covering an area of the a persons hand which is substantially only over the artery, so that when the air bag is being inflated with air, the air bag only presses down on the artery.

Another object of the present invention is to provide an air bag which provides a low AV/AVRTDSBP ratio, so that the largest possible APP to ABAV can be achieved.

Another object of the present invention is to provide a relatively small air bag which requires a relatively small volume of air to stop the blood flowing in the radial artery (i.e., the systolic blood pressure). (hereinafter referred to as “air volume required to determine systolic blood pressure” or AVRTDSBP), whereby the APP to AVRTDSBP ratio is relatively large. In other words, by using a small air bag to press down on the radial artery, the APP amplitude is relatively large when compared to the total air inside the air bag which is required to press down on the radial artery to a point where the blood in the radial artery stops flowing, i.e. systolic blood pressure, and, accordingly, provides a better APS as compared to if the air bag was big.

Another object of the present invention is to provide a diaphragm for an air bag for measuring blood pressure which has at least one oval shaped WAVE like protrusion so that when the diaphragm is inflated, the wave unfurls itself at a relatively low pressure, so that any slack between the diaphragm and a persons arm are taken up by the underling action of the wave;

Another object of the present invention is to provide a diaphragm for an air bag for an electronic blood pressure measuring device which when inflated, does not form any wrinkles along the surface thereof;

Another object of the present invention is to provide an electronic blood pressure measuring device having:

air bag stretching (hereinafter referred to as ABS) characteristics storing means for storing the air pressure required to inflate the air bag as a function of the air volume in the air bag; and

actual air bag pressure applied on the artery (hereinafter referred to as AABPOA) calculating means for calculating the actual pressure the air bag exerts on the artery

Another objective of the present invention is to provide an electronic blood pressure measuring device for an air bag which has a learning function incorporated therewith, whereby, the presently measured ACTUAL BLOOD PRESSURE are being compared with blood pressure measurements made in the past for the same patient which are stored in a RAM, and if three consecutive measurements are the same as or fall within a given range of previously made measurements, the air bag is instantly deflated, and the corresponding systolic and diastolic blood pressures value previously measured and stored in the RAM are displayed on the LCD of the blood measuring device, thereby eliminating any unnecessary discomfort by the patient, especially during the night.

Another object of the present invention is to provide a patient identification means for electronically identifying the patient to which the blood pressure measuring device is attached to so that not only the systolic and diastolic blood pressures are displayed on the electronic blood pressure measuring device but also the name of the respective name of the patient, thereby ensuring that no errors occur by the nurse in identifying the patient and registering information. Accordingly, each blood pressure measurement for each patient can be both stored in the electronic pressure measuring device, as well as transmitted to a central computer in the hospital. The stored and/or transmitted information can include include the name of the patient, the time, date, systolic, and diastolic blood pressures, etc.,

### BRIEF DESCRIPTION OF THE DRAWINGS

FIG. 1 shows a perspective view of a disposable air bag 10 according to the present invention mounted on a persons arm;

FIGS. 2A-2G show the parts and the assembly steps for making the air bag 10 of FIG. 1;

FIG. 2I shows a front view of an air bag 10A having a pocket for inserting a patient I.D. according to another embodiment of the present invention;

FIG. 2J shows a front view of an air bag 10B having male and female locking portions formed at the extending ends thereof according to another embodiment of the present invention;

FIG. 2K shows a side view of the male locking portion 1000m;

FIG. 2H shows a side view of ends of the air bags 10x-10y being folded;

FIGS. 3A-3E show a perspective view, a side view, a top view, a bottom view and a side cross sectional view at line II-II of FIG. 3C of a nipple 11 used in the air bag 10 of FIG. 1 according to the present invention;

FIG. 3F shows a side cross sectional view of another nipple 11B according to the present invention;

FIG. 4A-4D show a side view, a top view, a bottom view and a cross sectional view at line II-II of FIG. 4B of a nipple 111 according to another embodiment of the present invention;

FIGS. 5A and 5B show a front view and a side view of a diaphragm 12 according to a first embodiment of the present invention;

FIGS. 6A-6C show a top view, and cross sectional views at lines II-II and III-III in FIG. 6A of a diaphragm 120 according to another embodiment of the present invention;

FIG. 7 shows a schematic view of an air bag mounted on a persons wrist;

FIG. 8A-8D show a top view, a bottom view and cross sectional views at lines II-II and lines III-III of FIG. 8A of a diaphragm 1200 according to another embodiment of the present invention;

FIG. 9A-9D show a top view, a bottom view and cross sectional views at lines II-II and lines III-III of FIG. 9A according to another embodiment of a diaphragm 12000 according to the present invention;

FIG. 10A-10D show a top view, a bottom view and cross sectional views at lines II-II and lines III-III of FIG. 10A according to another embodiment of a diaphragm 12000 according to the present invention;

FIGS. 11A-11K show the parts and the manufacturing steps required to manufacture a disposable air bag 100000 according to another embodiment of the present invention;

FIG. 11L show top view of a disposable air bag 100000A according to another embodiment of the present invention;

FIG. 12A-12E show a perspective view, a side view, a top view, a bottom view, and a cross sectional view at line II-II of FIG. 12C of an air valve 110 according to another embodiment of the present invention;

FIG. 13A-13E shows a side view, a top view, a bottom view, a side cross sectional view at line II-II in FIG. 13C of an air valve 1100 according to another embodiment of the present invention;

FIG. 13G shows a bottom view of the nipple 1100B according to another embodiment of the present invention;

FIG. 14A-14E show a perspective view, a side view, a top view, a bottom view and a side cross sectional view at line II-II in FIG. 14C of a nipple 11000 according to another embodiment of the present invention;

FIG. 15A-15E show a perspective view, a side view, a top view, a bottom view, and a side cross sectional view at line II-II of FIG. 15C of a connector means 13 (hereinafter referred to as air valve connector 13 or clip 13) for hermetically connectively/disconnecting an air hose 14 to the nipple 11 or nipple 111 (shown in FIGS. 3 and 4) according to the present invention;

FIG. 15F shows a side view of an air hose 14 mounted on the clip 13;

FIGS. 16A-16E show a perspective view, a side view, a top view, a bottom view and a cross sectional view at line II-II of FIG. 16C of a clip 130 according to another embodiment of the present invention;

Numeral 16F shows a side view of the clip 130 having a hose 14 connected thereto;

FIGS. 17A-17E show a perspective view, a side view, a top view, a bottom view, and a side cross sectional view at line II-II of FIG. 17C of a clip 1300 according to another embodiment of the present invention;

FIG. 17F shows a side cross sectional view of a clip 1300B according to another embodiment of the present invention;

FIG. 18A-18D show a perspective view, a side view, a top view and a bottom view of a clip 1300F according to anther embodiment of the present invention;

FIG. 19A shows a perspective view of parts of a clamp 130000 according another embodiment of the present invention;

FIGS. 19B-19E show a side view with the clamp 130000 in the normally open position, a side view with the clamp 130000 in the closed position, a top view, and a bottom view of the clamp 130000;

FIGS. 20A-20E show a perspective view, a side view, a top view, a bottom view, and a cross sectional view at line II-II in FIG. 20C of a clip 13000 according to another embodiment of the present invention;

FIG. 20F shows a cross sectional view at line II-II in FIG. 20C of the clip 13000 with the nipple 11000 mounted therein;

FIG. 20G-20H show an end view and an end cross sectional view at line III-III in FIG. 20B of the clip 13000;

FIG. 20I shows an end cross sectional view at line III-III in FIG. 20B of the clip 13000 with the nipple 11000 mounted therein;

FIG. 21A shows a perspective view of a clip 13RF (hereinafter referred to as RF clip 13RF or clip 13RF) having a radio frequency reader (hereinafter referred to as RFR 130000RF) mounted therein for sending patient identification information to the electronic blood pressure measuring device to which the clip 13RF is connected to according to the present invention;

FIG. 21B-21E show a side view, a top view, a bottom view and a side cross sectional view at line II-II in FIG. 21C of the RF clip 13RF;

FIG. 21F, shows a front view of the RF clip 13RF;

FIG. 21G shows a cross sectional view at line III-III of FIG. 21B of the RF clip 13RF;

FIG. 21H shows a front view of the RF clip 13RF with the RFR 13000O RF mounted therein;

FIG. 21I shows a perspective view of a RFR 13000RF according to the present invention;

FIG. 21J shows a side cross sectional view at line II-II in FIG. 21C of the RF clip 13RF with the RFR 130000RF mounted therein;

FIG. 21K shows a plastic coupling device 15 for connecting the air hose 14 and the electrical wires 130000w to the electronic blood pressure measuring device (not shown) according to the present invention;

FIGS. 22A-22F show perspective view, a side view, a top view, a bottom view, a side cross sectional view at line II-II of FIG. 22C and a cross and a cross sectional view at line III-III of FIG. 22B of a unidirectional nipple 110000 according to another embodiment of the present invention;

FIGS. 23A show a perspective view of a stainless steel clip 26 according to another embodiment of the present invention;

FIG. 23B shows a front view of a sheet of steel 26P before being bent into the shape of the clip 26;

FIGS. 23C-23E show a side view, a top view and a bottom view of the clip 26 FIGS. 23F-23G show side view of the clip 26 in the open and closed states with a air hose attaching means 15 mounted therein and with an air valve 1100 mounted therein;

FIGS. 24A-24D show a a perspective view, a side view, a top view and a bottom view of an air hose/clip connector according to the present invention;

FIG. 25A shows a table of the measured air pressure inside an air bag as a function of the volume of air inside the air bag;

FIG. 25B shows a graph representative of the rubber diaphragm ABS characteristics values in the table of FIG. 25A;

FIGS. 26A-26D show front views of all parts needed to make the single decker air bag 100A according to another embodiment of the present invention;

FIGS. 26E-26H show the steps required to manufacture the single decker air bag 100A according to the present invention;

FIG. 26I shows a side cross sectional view at line II-II in FIG. 26H of the single deck air bag 100A;

FIGS. 27A-27F show front views of all the parts needed to make the double decker air bag 100B according to another embodiment of the present invention;

FIGS. 27G-27L show the steps required to manufacture the double decker air bag 100B according to the present invention;

FIG. 27M shows a schematic view of a double-deck air bag 100B shown in FIG. 27L wound around a persons wrist;

FIG. 27N shows a side cross sectional view of the air bag 100B at lines II-II of FIG. 27L;

FIGS. 28A-28C show the parts used in the manufacture of a stretchable air bag 1000A and the steps to manufacture the same according to another embodiment of the present invention;

FIGS. 28D and 28E show cross sectional views of the air bag 1000A at line II-II in FIG. 28C with no air and with air therein, respectively;

FIGS. 28F, 28G and 28H show three more embodiments of air bags 1000B, 1000C and 1000D according to the present invention;

FIGS. 28I and 28J show side views of the air bags 1000B-1000D with no air and with air inside the air bags 1000B-1000D, respectively;

FIG. 28K shows a side cross sectional view of an air bag 1000E according to another embodiment of the present invention;

FIG. 28L shows a side cross sectional view of an air bag 1000F according to another embodiment of the present invention;

FIG. 29A show a front view of a rectangular shaped film sheet of plastic material L5 having a plurality of flexible semi round protrusions B1;

FIG. 29B shows a side cross sectional view at line II-II in FIG. 29A of the sheet L5;

FIG. 29C shows a front view of a double decker air bag 100C having the bubble sheet L5 as the outermost sheet;

FIG. 30A shows a perspective view of a disposable air pressure belt 17 having a diaphragm 12000N and electronic pressure measuring device 18 mounted therein according to an embodiment of the present invention;

FIG. 30B-30E show a side view, a top view, a bottom view and a side cross sectional view at line II-II in FIG. 30C of a bendable but not stretchable band 17 for a blood pressure measuring device according to the present invention;

FIG. 30F shows a cross sectional view of the belt 17 at line II-II in FIG. 30C having a light emitting diode LED 71 and a photo sensor 72 mounted therein;

FIG. 30G shows a cross sectional view of the belt 17 at line II-II of FIG. 30C further having a diaphragm 1200ON and an electronic blood pressure measuring device 18 mounted therein;

FIGS. 30H, 30I show a front view and a back view of the disposable air pressure belt 17 having a diaphragm 12000N and electronic pressure measuring device 18 mounted therein;

FIG. 30J shows a side cross sectional view of the belt at line II-II of FIG. 30H having a diaphragm 12000N and an electronic blood pressure measuring device 18 mounted therein, the belt 17 being bent in a circle;

FIG. 31A-31D show a front view, a back view and cross sectional views at lines II-II and III-III of a diaphragm 12000N according to another embodiment of a diaphragm according to the present invention;

FIG. 32A, 32B show side views of a conventional light emitting diode LED 71 and a photo sensor 72;

FIG. 33A-33D show a front view, a back view, an side view and a cross sectional view at line II-II in FIG. 33B of a plastic box for containing an electronic blood pressure measuring device;

FIG. 34A shows a perspective view of a band 170 having a blood pressure measuring device 18, a diaphragm 12000N and a manual air pressure pump 180 mounted therein according to another embodiment of the present invention;

FIGS. 34B-34C show, a front view and a side view of the bendable but not stretchable band 170 shown in FIG. 34A;

FIG. 34D shows a side cross sectional view of the band 170 having a blood pressure measuring device 18, a diaphragm 12000N and a manual air pressure pump 180 mounted therein, the band 17 being in a wound state;

FIGS. 34E-34G show a perspective view, and side cross sectional views at lines II-II and III-III in FIG. 35E of a manual rubber air pump 180 according to the present invention;

FIG. 35A-35D show a side view, a front view, a back view and a side cross sectional view at line II-II in FIG. 35C of a pre-stretch diaphragm 77 according to another embodiment of the present invention;

FIG. 36A and FIG. 36B show a front view and a side view of an oval shaped ring 78;

FIG. 37A shows a side cross sectional view of the diaphragm 77 mounted on the ring 78;

FIG. 37B shows a side cross sectional partial view of the band 17 where the cavity 17c is formed with no diaphragm inserted therein;

FIG. 37C shows a side cross sectional view of the diaphragm 77 mounted on the ring 78 which are then together mounted inside the oval groove 17g in the cavity 17C in the band 17;

FIGS. 39A-39D show a side view, a top view, a bottom view and a cross sectional view at line II-II in FIG. 39C of a nipple 39 for an air bag according to another embodiment of the present invention;

FIGS. 39E-39H show a side view, a top view, a bottom view and a cross sectional view at line II-II in FIG. 39G of a connector 49 for connecting and disconnecting an air hose 14 to and from the nipple 39 according to another embodiment of the present invention;

FIGS. 39I-39K show a side view, a top view and a bottom view of a rubber cap 59 for blocking water from entering through the nipple 39;

FIG. 39L shows a side cross sectional view of the connector 49 mounted in the nipple 39;

FIG. 39M shows the nipple 39 mounted in an air bag 100000A according to another embodiment of the present invention;

FIG. 39N-39R show a perspective view, a side view, a top view, a bottom view and a cross sectional view at line II-II of FIG. 39P of a nipple 79 according to another embodiment of the present invention;

FIG. 39S shows a cross sectional view of the nipple 79 shown in FIG. 39P having the rubber cap 59 mounted thereon;

FIG. 39T shows a cross sectional view of the nipple 79 shown in FIG. 39P having the rubber cap 59 mounted thereon and the connector 49 mounted therein;

FIG. 40 shows a block diagram of an electronic blood pressure measuring device 101 according to the present invention;

FIG. 41A, 41B show a FLOW CHART 1 and FLOW CHART 2 for determining the systolic and diastolic blood pressures as a function of the air bag stretching characteristics;

FIG. 42 shows a block diagram of an electronic blood pressure measuring device 102 according to another embodiment of the present invention;

FIGS. 43A and 43B show another embodiment of a FLOW CHART 3 and FLOW CHART 4 for determining the systolic and diastolic blood pressures according to the present invention;

FIGS. 44A, 44B and 44C show subroutines for “RELEASE AIR IN THE AIR BAG MODE”, “VACUUM AIR BAG MODE” and “PUMP MODE” of operation;

FIG. 45 shows FLOW CHART 8 for measuring the systolic and diastolic blood pressure while not requiring the air volume measuring device 34;

FIGS. 46A-46F show the parts and the steps to manufacture a multi-air-bag-band 333 according to another embodiment of the present invention;

FIG. 47A, 47B show perspective views of an air hose 140 comprising three air hoses 140A, 140B and 140C integrally formed with each other;

FIG. 48 shows a front view of a multi-air-bag-band 333A according to another embodiment of the present invention;

FIG. 49A shows a perspective view of a multi-clip 133 according to the present invention;

FIGS. 49B-49D show a bottom view, a back view and a front view of the multi clip 133;

FIG. 50 shows a block diagram of a multi-air-bag electronic blood pressure measuring device 103 according to another embodiment of the present invention; and

FIG. 51 shows a graph of measured air pressure (MAP) in the air bags 31A, 31B and 31C as a function of time.

### DETAILED DESCRIPTION OF THE PREFERRED EMBODIMENTS

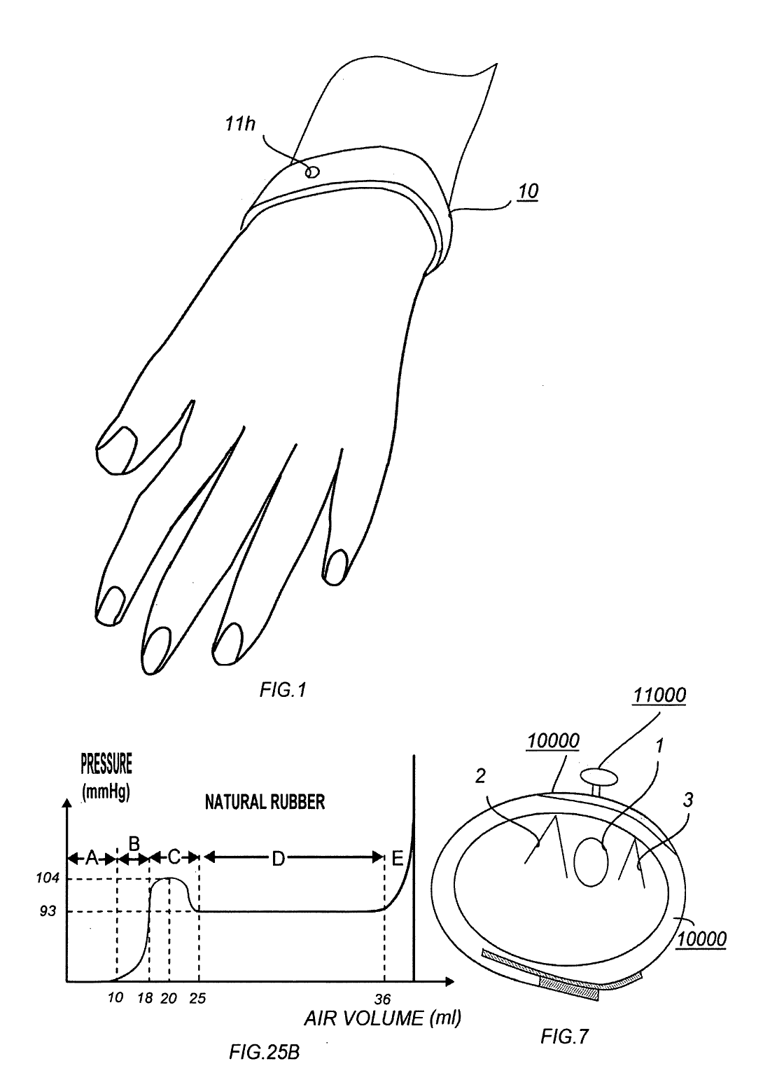

FIG. 1 shows a perspective view of a disposable air bag according to the present invention mounted on a persons arm.

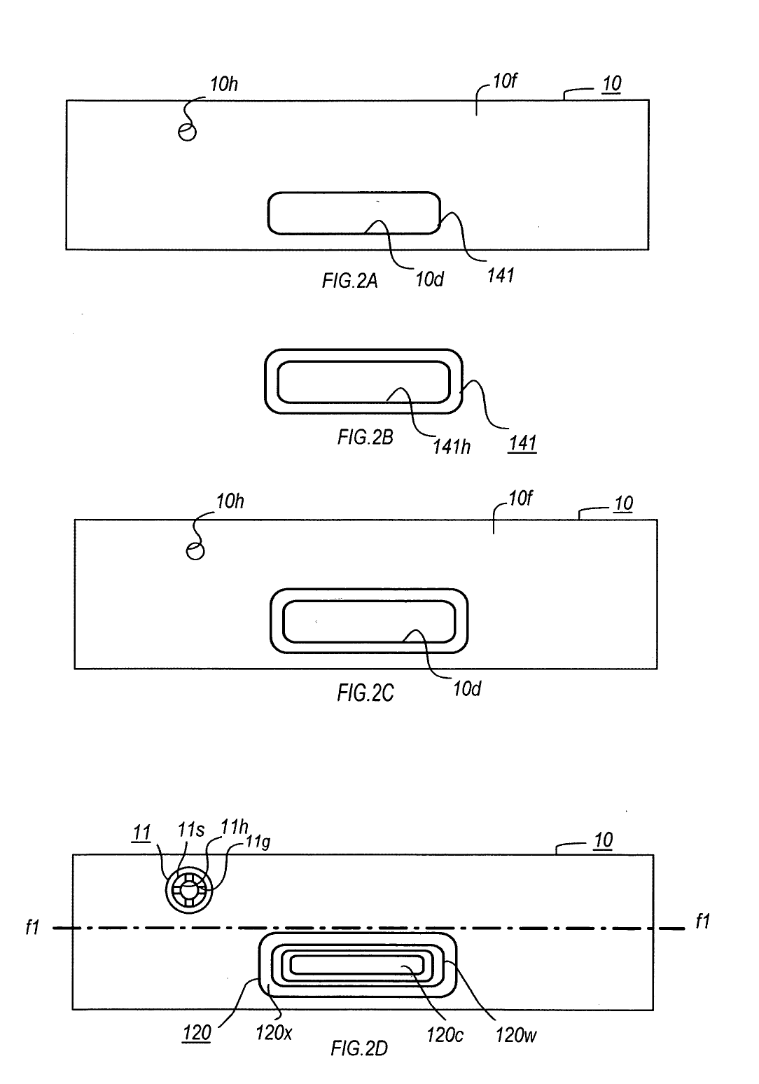

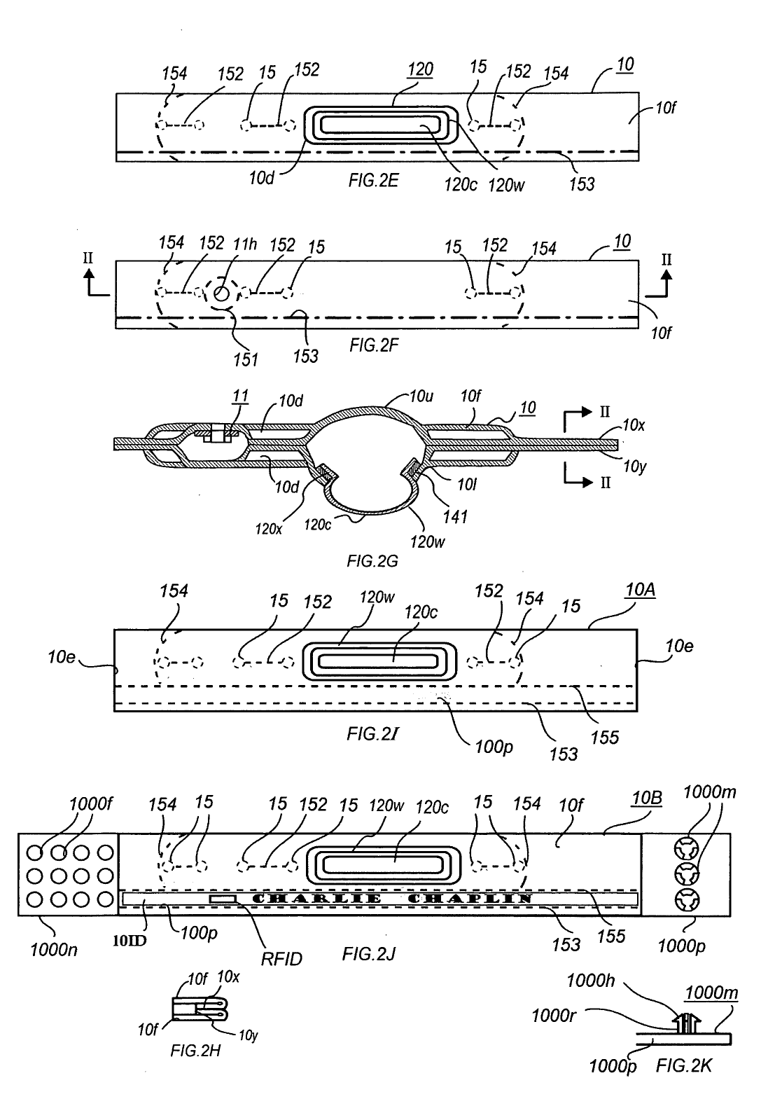

FIGS. 2A-2G show the parts and the assembly steps for making the air bag 10 of FIG. 1.

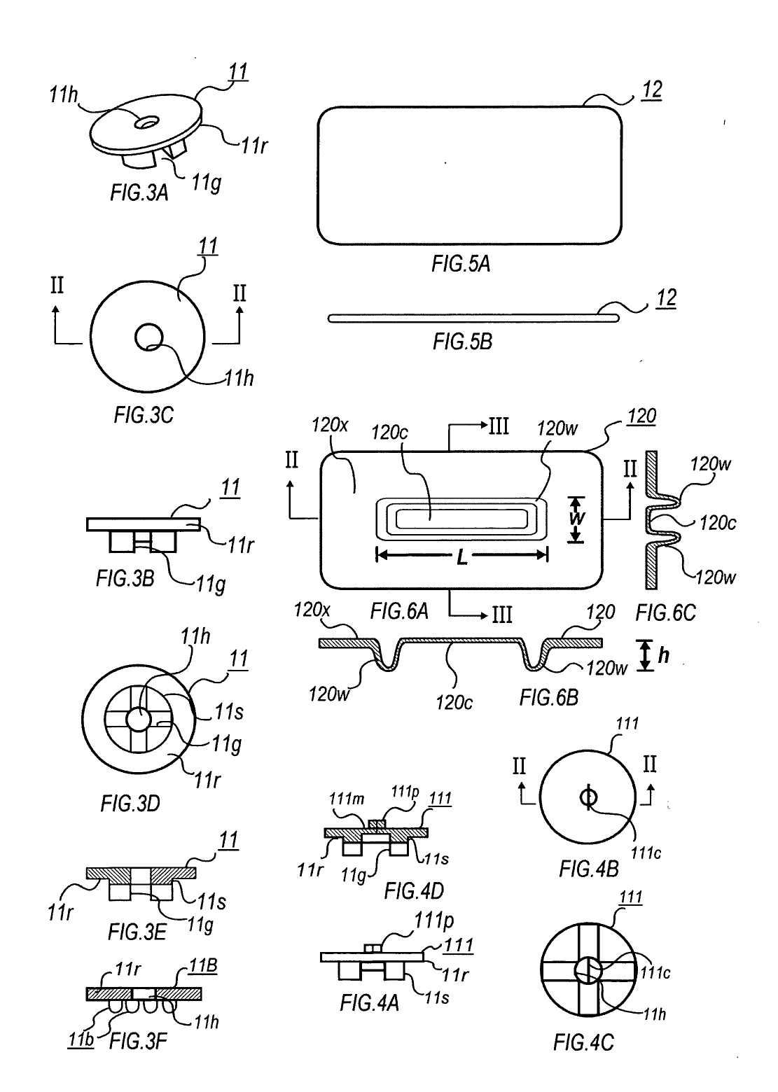

FIGS. 3A-3E show a perspective view, a side view, a top view, a bottom view and a side cross sectional view at line II-II of FIG. 3C of a nipple 11 used in the air bag 10 of FIG. 1 according to the present invention.

Referring to FIGS. 3A-3E, numeral 11 generally designates an air valve (hereinafter referred to as a nipple 11) according to the present invention. Numeral 11s designates a round shaft having a hole 11h formed through the center thereof in the axial direction of the shaft 11. Numeral 11r designates a thin ring integrally formed with the shaft 11s along the outer periphery of the shaft and along an upper end thereof. Numeral 11g designates four radial grooves extending from the hole 11h to the periphery of the shaft 11s formed along a lower end of the shaft 11.

FIG. 4A-4D show a side view, a top view, a bottom view and a cross sectional view at line II-II of FIG. 4B of a nipple 111 according to another embodiment of the present invention. The nipple 111 is similar to the nipple 11 and only the differences therebetween will be described herebelow.

Referring to FIGS. 4A-4D, numeral 111m designates a thin membrane integrally formed with the nipple 111 along the upper end thereof which hermetically seals the hole 11h along the upper end of the nipple 111. Numeral 111p designates a round protrusion formed at the center of the membrane 111m. The protrusion 111p

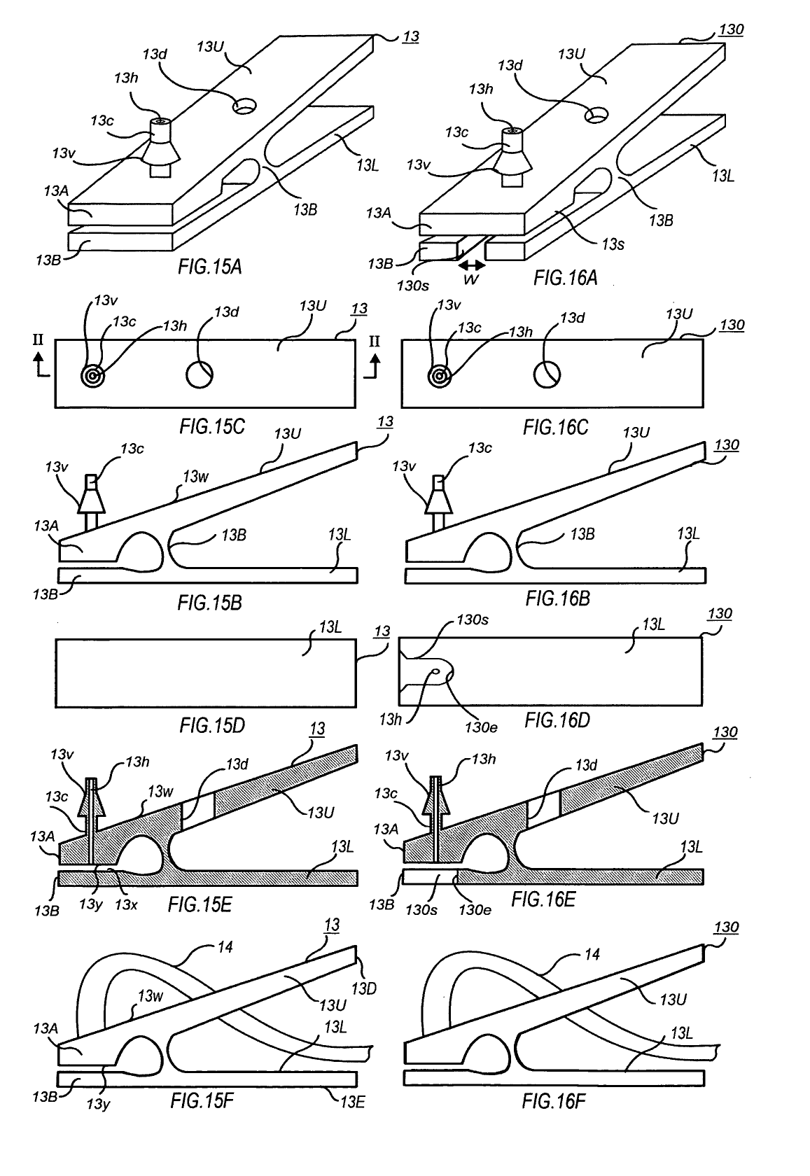

protrudes above the upper surface of the nipple 111. The nipple 111 is formed using conventional injection molding techniques and is made of rubber, latex, polypropelene, enca vynil, or any other flexible material. Numeral 111c designates a cut made by a sharp cutter along the center of the nipple 111. The protrusion 111p normally prevents water from flowing through the nipple 111, thereby preventing water from entering the air bag in which it is mounted in. When a clip (i.e. clip 13 shown in FIG. 15) is mounted on the nipple 111, the protrusion 111p is pressed downwards into the hole 11h and slightly opens the hole 11h thereby allowing air to flow through the nipple 111. The outer diameter of the protrusion 111p is smaller than the diameter of the hole 11h, so that it may fit inside the hole 11h when pressed down by a clip 13 (as will be described herebelow). Furthermore, the length of the cut 111c is the same as the inner diameter of the hole 11h.

The reason for the grooves 11g is to make sure that air flow through the nipples 11 and 111 is not interrupted during the inflation or deflation of air in the air bag in which the nipple 11 and 111 are mounted in (i.e. due to the bottom surface of the shaft 11s coming into contact with the inner wall of the film used to make the air bag in which the nipple 11, 111 are mounted in).

FIG. 3F shows a side cross sectional view of another nipple 11B according to the present invention. The nipple 11B is similar to the nipple 11 but does not have the shaft 11s. Referring to the FIG. 3F, the nipple 11B comprises a thin disc 11r having a round hole 11h formed through the center thereof and a plurality of round bumps 11b formed on the bottom surface thereof. This nipple 11B is much easier to manufacture and is thinner while still providing the same characteristics as the nipple 11.

Another embodiment of the nipple 11, 111 can, instead of having the grooves 11g formed in the bottom of the shaft 11s, have the bottom surface of the shaft formed with small bumps (i.e. the bottom of the shaft 11s should not be a smooth surface)

FIGS. 5A and 5B show a front view and a side view of a diaphragm 12 according to a first embodiment of the present invention.

Referring to FIGS. 5A, 5B, numeral 12 generally designates a diaphragm which comprises a rectangular piece of stretchable material which is made of silicone, rubber, latex or any other elastic material. The diaphragm 12 is much more stretchable than the rest of the air bag in which it is mounted. For example, when the diaphragm 12 is made from natural rubber (i.e. bendable and stretchable material) and the rest of the air bag (as will be described herebelow) is made from polyethylene (i.e. bendable but not stretchable), the air bag and diaphragm will first fill up with air and then the diaphragm 12 will stretch outwardly towards the radial artery, whereby a much more accurate systolic and diastolic measurement can be achieved. However, even with this combination of rubber for the diaphragm and polyethylene for the air bag, if the patient wears the air bag loosely around their wrist, to measure the systolic and diastolic blood pressures, the rubber diaphragm must be inflated sufficiently to reach out towards the radial artery and then apply sufficient pressure against the radial artery. Accordingly, two sets of forces are required, one to stretch the diaphragm 12 towards the radial artery and the other to press the diaphragm 12 against the radial artery. Accordingly, it is not possible to correctly measure the systolic and diastolic blood pressures, since it is not possible to know exactly how much the diaphragm is being stretched (i.e. the diaphragm will be stretched less for tightly mounted air bags and stretch more for loosely mounted air bags). In order to overcome this drawback, a diaphragm requiring relatively little or no force to expand outwardly when filled with air is desirable. One way to solve this problem is to make one or more concentric waves along the surface of the diaphragm 12, so that as air is pumped into the diaphragm, the waves unfurl and allow the diaphragm to move effortlessly outwards towards the radial artery, and, accordingly, only the force to press against the radial artery is required. This problem is further compounded by the fact that to reach the radial artery 1, the diaphragm being used must navigate around the radius 2 and the digital tendon 3, which basically dictates that the diaphragm must not only be bendable but also very stretchable to be able to maneuver around these obstacles or barriers (i.e. the radius 2 and the digital tendon 3). Since the average distance between the radius 2 and the digital tendon 3 is about 10 mm, it does not leave much room to maneuver.

FIGS. 6A-6C show a top view and cross sectional views at lines II-II and III-III in FIG. 6A of a diaphragm 120 according to another embodiment of the present invention. The diaphragm 120 is similar to the diaphragm 12 and only the differences therebetween will be described herebelow Numeral 120w designates a wave-like elliptical protrusion (hereinafter referred to as a wave portion 120w or wave 120w) formed along a central part of the diaphragm 120. Numeral 120c designates the portion of the diaphragm inside the wave 120w (hereinafter referred to as central portion 120c) and numeral 120x designates the portion of the diaphragm outside the wave 120w (hereinafter referred to as the outer portion 120x). The length L and width W of the wave 120w are preferably 50 mm and 20 mm, respectively, so that the wave 120w and central portion 120c can be easily positioned over the radial artery 1. The height h of the wave 120w is preferably 5 mm and the pitch of the wave should preferably be about 1-5 mm., so that the central portion 120c, inside the elliptical wave 120h, when inflated, can easily move outwards of the air bag that it is mounted in, as will be explained in more detail herebelow.

The wave portion 120w, the central portion 120c and the outer portion 120x are integrally formed with each other using conventional molding techniques, or dipping techniques (i.e. the way condoms are manufactured).

Although only one wave 120w is shown, a plurality of concentric waves can be formed around each other to allow the central portion 120c to move outwardly easily when the air bag to which the diaphragm 120 is attached to is inflated with air. The outer portion 120x is used for mounting the diaphragm to an air bag as will be described herebelow.

The diaphragm 120 is made of rubber, latex, silicon or any other stretchable material using conventional injection molding techniques or dipping techniques as is commonly used in the manufacture of balloons and condoms.

Preferably, the thickness of the diaphragm 120 should be thinnest along the center of the central portion 120c of the diaphragm 120 and the thickness should gradually increase from the center of the diaphragm 120c to the wave 120w. By gradually increasing the thickness of the diaphragm 120 from the inner central portion 120c outwards, when the diaphragm 120 is inflated, the central part 120c will expand outwardly first followed by the wave 120w (i.e. the wave 120w will unfurl). This unfurling action of the wave 120w will take up any slack between the air bag in which the diaphragm 120 is mounted in and the patients arm. Furthermore, as the diaphragm 120 continues to be filled with air, when the central portion 120c of the diaphragm 120 starts pressing against a persons arm over the location where the radial artery is located, as the air pressure increases inside the air bag in which the diaphragm 120 is mounted, the wave 120w will also press against the persons skin around the central portion 120c, thereby providing a physical barrier (i.e. like a elliptical dam) preventing the central portion 120c from expanding laterally sideways along the persons arm (hereinafter referred to as diaphragm lateral escape blocking means or DLEBM), and thereby making sure that the central portion 120c of the diaphragm 120 can only expand (i.e. press) radially outwards towards the radial artery.

Preferably, the central portion 120c and the wave portions 120w should be formed in an elliptical shape having a length with the elliptical central portion being about 3-5 cm. long and 2-4 cm. wide, so that it can easily be positioned over the radial artery 1. Furthermore, preferably, the thickness of the oval central portion 120c should have a central oval area inside the oval central portion 120c which is uniform in thickness, the central oval area being thinner than the rest of the central portion 120c, so that when the diaphragm 120 is inflated with air the central area inside the central portion 120c does not expand outwardly as a round shaped ball, but like an American style football, thereby providing a diaphragm 120 which is less “POSITION SENSITIVE” when mounting the diaphragm over the radial artery. Accordingly, regardless of the POSITION which the diaphragm is mounted around the radial artery 1, as long as any of the central part of the central portion 120c is located over the radial artery 1, the same systolic and diastolic measurements should be obtained, thereby making the diaphragm “less position sensitive”. The thickness of the central portion 120c around the central area of the central portion 120c should uniformly gradually increase towards the wave portion 120w. The central oval area should preferably be about 80 percent the size of the central portion 120c.

FIGS. 2A-2G show the parts required to make a disposable air bag 10 and a method of making the same according to a first embodiment of the present invention.

FIG. 2A shows a front view of thin film 10f which is made from a material which is easily bendable but not stretchable, such as polyethylene, etc. having a thickness of about 0.03-0.1 mm. The film 10f is cut into a rectangular shape having a length of about 60 cm. (i.e. long enough to fit around a persons arm) and a width of about 6 cm. The film 10f has an air passage hole 10h and a diaphragm hole 10d cut or punched therethrough. The holes 10h and 10d are formed on opposite sides of the film 10f with respect to the width thereof, so that when the film 10f is folded in half along the length of the film 10f (i.e., as shown by the dot and dash line f1-f1 in FIG. 2D), the holes 10h and 10d are on opposite sides of the folded film 10f. Furthermore, the hole 10d is made around the center of the film 10f with respect to the length thereof, while the hole 10h is made near one end (i.e. about 4 cm) from the end of the film with respect to the length of the film 10f.

FIG. 2B shows a top view of a double sided tape 141 (hereinafter referred to as DST 141). The outer size and shape of the DST 141 is the same as the outer dimensions of the diaphragm 120. The DST 141 further has a central through hole 141h which is the same size and shape as the hole 10d in the film 10f.

FIG. 2C shows the DST 141 glued to the inner side of the film 10f around the diaphragm hole 10d.

FIG. 2D shows a front view of the thin film 10f further having the nipple 11 and the diaphragm 120 mounted in the holes 10h and 10d, respectively. The nipple 11 is connected to the film 10f by heat sealing (shown by a round dash line 151 in FIG. 2F) the nipple 11 to the film 10f along the periphery of the ring 11r. Alternatively, the upper surface of the nipple 11 may be mounted on the film 11 using double sided tape. (not shown) similarly to the way the diaphragm 120 is mounted to the film 12f.

Next, the double sided tape 141 shown in FIG. 2B is mounted on the film 10f with the holes 141h and 10d aligned on top of each other (i.e. as shown in FIG. 2C).

Next, as shown in FIG. 2D, the diaphragm 120 is mounted inside the hole 10d in the film 10f and the outer portion 120x of the diaphragm 120 is bonded to the upper surface of the double sided tape 141. The central portion 120c and the wave 120w are positioned above the hole 10d in the film 10f, so that the wave 120w and central portion 120c are free to move out of the hole 10d in the film 10f. The wave 120w preferably faces upwards, so that when the film 10f is folded in half along the length thereof, the wave 120w faces towards the film portion of the film 10f where the nipple 11 is mounted (hereinafter referred to as the outer portion of the film 10f).

The hole 10d should have the same shape and the same size as the outer diameter of the wave 120w, so that the wave 120w can freely expand (i.e. stretch) outwards of the film 10f when the air bag 10 is inflated through the nipple 11.

Next, the film 10f is folded in half along the length thereof (i.e. along line f1-f1 in FIG. 2D) and then heat sealed along dash lines 152-154 as shown in FIG. 2F.

Numerals 15 designate round heat seals (hereinafter referred to as air pressure spots 15) which are respectively formed at the respective ends of each of the heat welds 152. These pressure spots 15 serve to distribute the stress at the ends of the heat welds 152 due to air pressure inside the air bag 10.

FIG. 2G shows cross sectional view of the disposable air bag 10 at lines II-II of FIG. 2F, in the air bag 10 inflated mode. Referring to the Fig., it can be seen that the central part of the air bag 10 where the diaphragm 120 is mounted has the largest diameter, and the parts of the bag 10 where the heat welds 152 are made are smaller in diameter. Each of the welds 152 causes the bag 10 to inflate in the shape of two hotdogs on either side of the weld 152.

FIG. 2H shows a partial view at line II-II in FIG. 2G. Referring to the FIG. 2G, it can be seen that the ends 10x, 10y of the film 10f are first folded inwards before the heat sealing 153 is done (i.e. the heat seal 153 hermetically joins four layers of film 10f). In this way, there are no sharp edges present in the air bag 10 which, otherwise, may annoy or irritate the patient that the air bag 10 is mounted on.

FIG. 2I shows a front view of an air bag 10A according to another embodiment of the present invention. The air bag 10A is similar to the air bag 10 and only the differences therebetween will be described herebelow.

The air bag 10A further includes a heat weld 155 which extends the whole length of the folded film 10f about 10 mm above the heat seal 153 and runs parallel therewith. These heat welds 153 and 155 create a pocket 100p which allows a name tag (i.e. a piece of paper with the name CHARLIE CHAPLIN printed on it as an example of a patient) to be slid into the pocket 100p through either side of the pocket between the heat welds 153 and 155,

The ends of the air bags 10e can have male and female Velcro parts joined thereto using double sided tape, so that the air bag can be wound around a patients arm and then the Velcro parts jointed to each other.

Another method of joining the ends of the air bag 10e around a patients arm is by having a nurse wind the air bag 10 or 10A around the patients arm and then using a heat sealing device (not shown but well known in the art of sealing plastic bags by electrically heating a micron wire) to join the ends 10, 10A to each other. In this way, the patient cannot remove the air bag from his arm, unless it is cut off. With this method of heat sealing and joining the ends of the air bags 10 or 10A around a patients arm, the paper having his name printed on it is permanently sealed inside the pocket 100p. The film 10f must be transparent in this case, so that the patients name is visible from outside the air bag 10, 10A.

FIG. 2J shows another embodiment of an air bag 10B according to the present invention. The air bag 10B is similar to the air bag 10 and only the differences therebetween will be described herebelow.

Referring to FIG. 2J, numerals 1000n and 1000p designate two rectangular strips of bendable but not stretchable plastic material such as polyethylene which are thicker than the film 10f. One end of each of the strips 1000n and 1000p are respectively heat sealed to a respective end of the air bag 10B. Numeral 1000f and 1000m designate a plurality of female (i.e. 12 are shown in the Fig.) and plurality of male (i.e. 3 are shown in the Fig.) locking portions which are formed in the strips 1000n and 1000p according to the present invention. The male and female locking portions permanently lock into each other when they are pressed together. The female portions comprise a plurality of rows of three holes adjacent to each other.

FIG. 2K shows a side view of the male locking portion 1000m. Referring to the Fig., the male portion comprises three self locking portions integrally formed therewith. Each of the male locking protrusions comprises three legs 1000r each having an outwardly facing triangular head 1000h integrally formed therewith along the extending end thereof. When the row of male portions 1000m is pressed into a row of female portion 1000f, the legs 1000r bend inwards to allow the arrow portions 1000h to pass through the holes 1000f and then to permanently lock the legs 1000r in the holes 1000f. The row of female portions 100f chosen for the row of male portions 1000m is the row that allows the air bag 10B to loosely fit around a patients arm. The reason for having rows of three male and female locking portions is to make sure that the pulling stresses generated when the air bag 10B is inflated on the male and female portions 1000m, 1000f are evenly distributed along the width of the air bag 10B.

Diaphragm Tow Waves No Wall

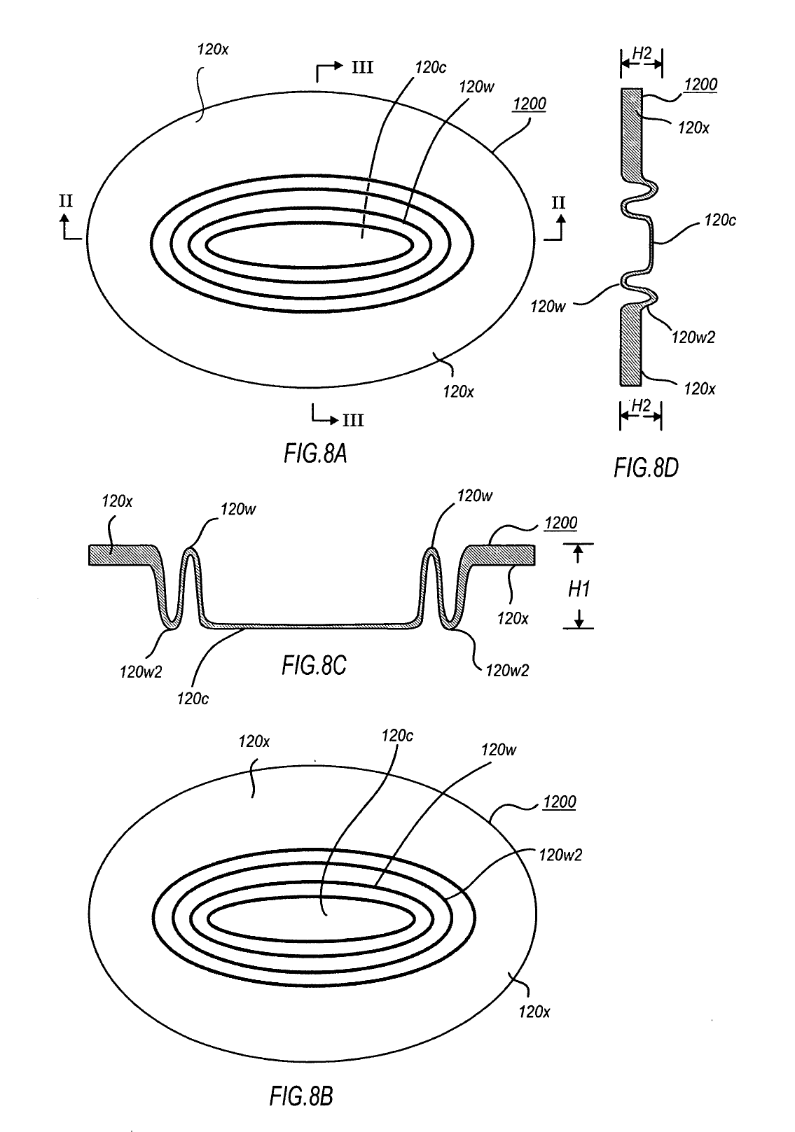

FIG. 8A-8D show a top view, a bottom view and cross sectional views at lines II-II and lines III-III of FIG. 8A according to another embodiment of the diaphragm 1200 according to the present invention. The diaphragm 1200 is similar to the diaphragm 120 and only the differences therebetween will be described herebelow.

Referring to FIGS. 8A-8D, numeral 120w2 designates an outer wave concentrically formed around the inner wave 120w, and numeral 120c designates a central oval portion. With this diaphragm 1200, having the waves 120w and 120w2 allows the central portion 120c to move out even further with relatively little air pressure inside the air bag in which the diaphragm 1200 is mounted in. Accordingly, with this diaphragm 1200, even larger amounts of slack between the diaphragm and a patient hand can be compensated for. Preferably, the waves 120w and 120w2 are made 5 mm high. In this case when the waves 120w, 120w2 unfurl, the central portion 120c protrudes 5×3=15 mm out of the diaphragm.

It should be noted that the height of the waves 120w and 120w2 and the pitch of the waves 120w and 120w2 can be varied along the length thereof (i.e. as shown by numeral H1 and H2 in FIGS. 8C and 8D, respectively), so that as the diaphragm 120 is inflated the diaphragm easily adjusts to the contour of the persons hand on which it is mounted on, so that no wrinkles form along the central portion 120c of the diaphragm 120.

Diaphragm Two Rings and Wall

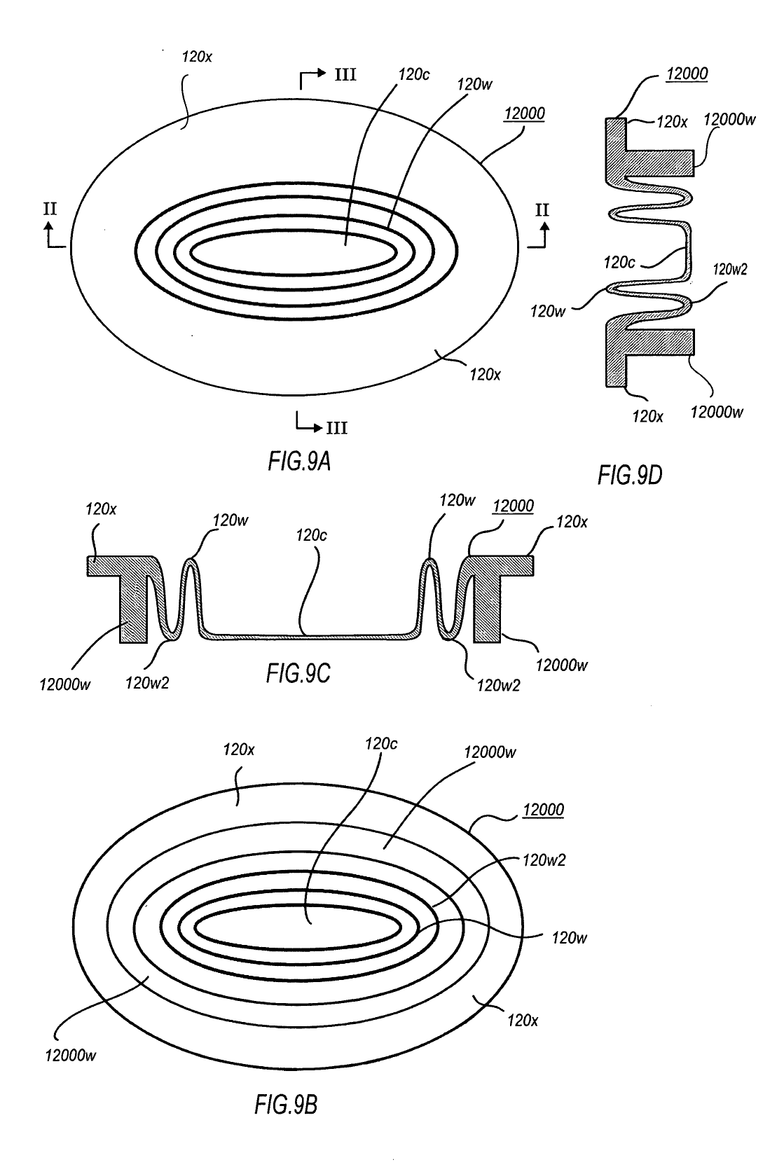

FIG. 9A-9D show a top view, a bottom view and cross sectional views at lines II-II and lines III-III of FIG. 9A according to another embodiment of a diaphragm 12000 according to the present invention. The diaphragm 12000 is similar to the diaphragm 1200 and only the differences therebetween will be described herebelow.

Referring to FIGS. 9A-9D, numeral 12000w designates a wall formed around the outer wave 120w2. The height of the wall 12000w is the same as or slightly higher than the height of the waves 120w and 120w2.

Preferably, the thickness of the diaphragm 12000 should be thinnest along the center of the central part 120c and gradually increasing in thickness from the central portion 120c to the inner most wave 120w and then to the outer wave 120w2 of the diaphragm 12000. By gradually increasing the thickness of the diaphragm 1200 from the inner central part 120c outwards, when the diaphragm is inflated, the central part 120c will expand outwardly first followed by the inner wave 120w and then the outer wave 120w2 (i.e. the waves will unfurl). Furthermore, as the diaphragm 12000 continues to be filled with air, when the central part 120c of the diaphragm 12000 starts pressing against a persons arm (i.e. skin) over the location where the radial artery 1 is located, as the air pressure is increased inside the air bag in which the diaphragm 12000 is mounted in, the waves 120w2 and 120w begin to press against the persons skin around the central part 120c of the diaphragm 12000, thereby providing a physical barrier (i.e. like a round dam) preventing the central part 120c from expanding laterally sideways (hereinafter referred to as diaphragm lateral escape blocking means or DLEBM), and thereby making sure that the central part 120c of the diaphragm 12000 can only expand and, accordingly, press radially outwards toward the radial artery 1.

The wall 12000w serves three major functions.

1. When the air bag in which the diaphragm 12000 is deflated (i.e. the air bag 10), the waves 120w and 120w2 begin to furl up (i.e. like an accordion) and the upper and lower walls 10U and 10L (in FIG. 2F) start to move towards each other. The wall 12000w ensures that the waves 120w and 120w2 can neatly furl up into the air bag 10 without bumping into the upper wall 10u (i.e. shown in FIG. 2G). Otherwise, the waves 120w2 and 120w and the central part 120c of the diaphragm 12000 may cause discomfort and irritate the skin of the patient wearing the air bag 10, since, without the wall 12000w, the waves 120w and 120w2 may be all ruffled up and partially sticking out of the air bag 10.

2. The wall 12000w substantially restrict the axial and radial movement of the outer portion 120x of the diaphragm 12000 from moving outwardly of the wall 12000w when the air bag in which the diaphragm 12000 is mounted in is being inflated with air. However, the central part 120c and the waves 120w and 120w2 are free to move outwardly towards the radial artery. This will provide extra support around the hole 10d of the air bag in which the diaphragm is mounted in. For example, in the case of the air bag 10, the hole 10d in the film 10f, provided for the diaphragm 12000, will have less radial and axial stresses exerted on it as the waves 120w and 120w2 of the diaphragm 12000 expand outwardly and increase in size as the air bag 10 is inflated with air.

3. Since the wall 12000w is relatively stiff (i.e. due to its thickness t1 of the wall 12000w being much greater than the thickness of the central portion 120c and waves 120w2 and 120w), when the surface of the film 10f of the air bag 10 in which the diaphragm 12000 is mounted in is pressed towards the skin above the radial artery, the radius bone 2 and the digital tendon 3 (hereinafter referred to as two pillars or pillars 2,3) will prevent the wall 12000w from moving towards the radial artery 1 (i.e. the wall 12000w will act like a bridge supported on the pillars 2,3), whereby, the pillars, rather than being a hindrance or a block for the diaphragm 12000, the pillars 2 and 3 act as pillars to support the wall 12000w while allowing the most flexible and stretchable central part 120c to expand and penetrate into the hand between the pillars 2 and 3 towards the radial artery 1. Furthermore, the wall 12000w, prevents the film 10f from pressing down towards the radial artery 1, which otherwise, would interfere with (i.e., partially block) the blood flow in the radial artery 1, and since the film 10f is not stretchable (and therefore not as sensitive as the central portion 120c of the diaphragm 12000 is to changes in blood pressure BPP inside the radial artery 1), the film 10f would interfere with the central portion 120c providing a faithful and true replication of the blood pulse BPP in the radial artery. In other words, with this structure of the diaphragm 12000, it is guaranteed that the best transformation of the blood pressure (hereinafter referred to as blood pressure or BP) and changes in blood pressure due to blood pulses inside the radial artery (hereinafter referred to as blood pressure pulses or BPP) to corresponding air pressure (hereinafter referred to as air pressure or AP) and changes in air pressure (hereinafter referred to as air pressure pulses or APP) inside the air bag 10 will be provided at a very high resolution.

Diaphragm with 2 Waves, Wall and Ribs and Lip

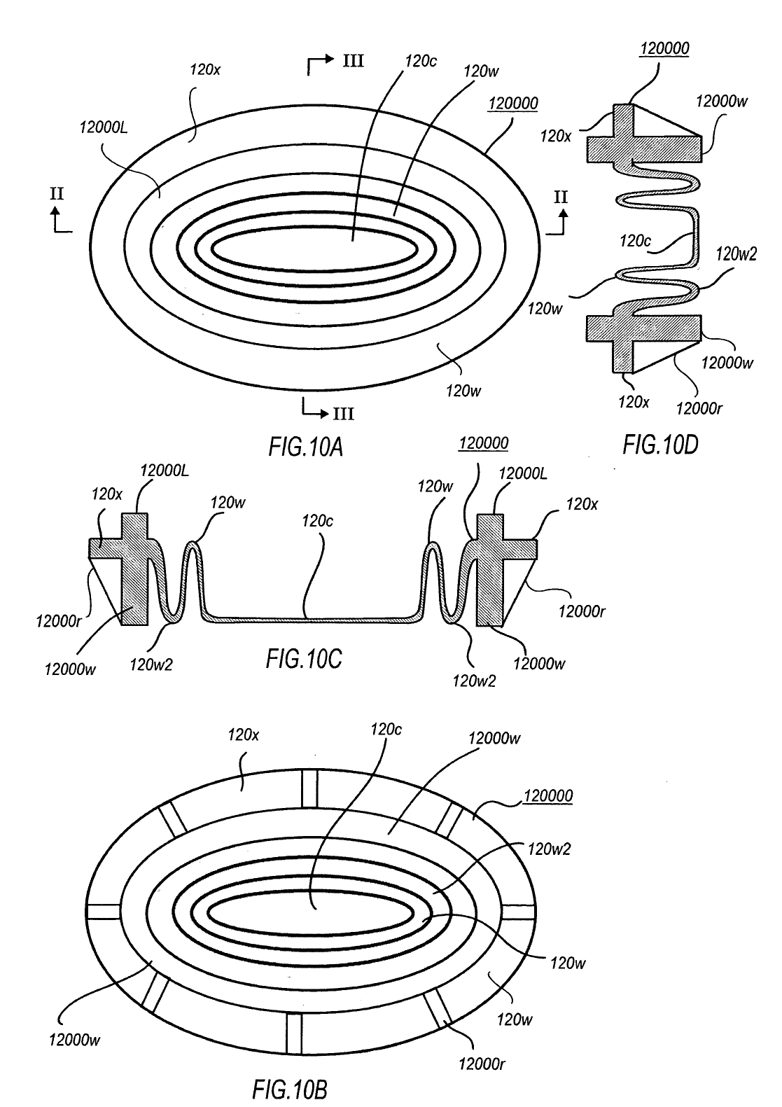

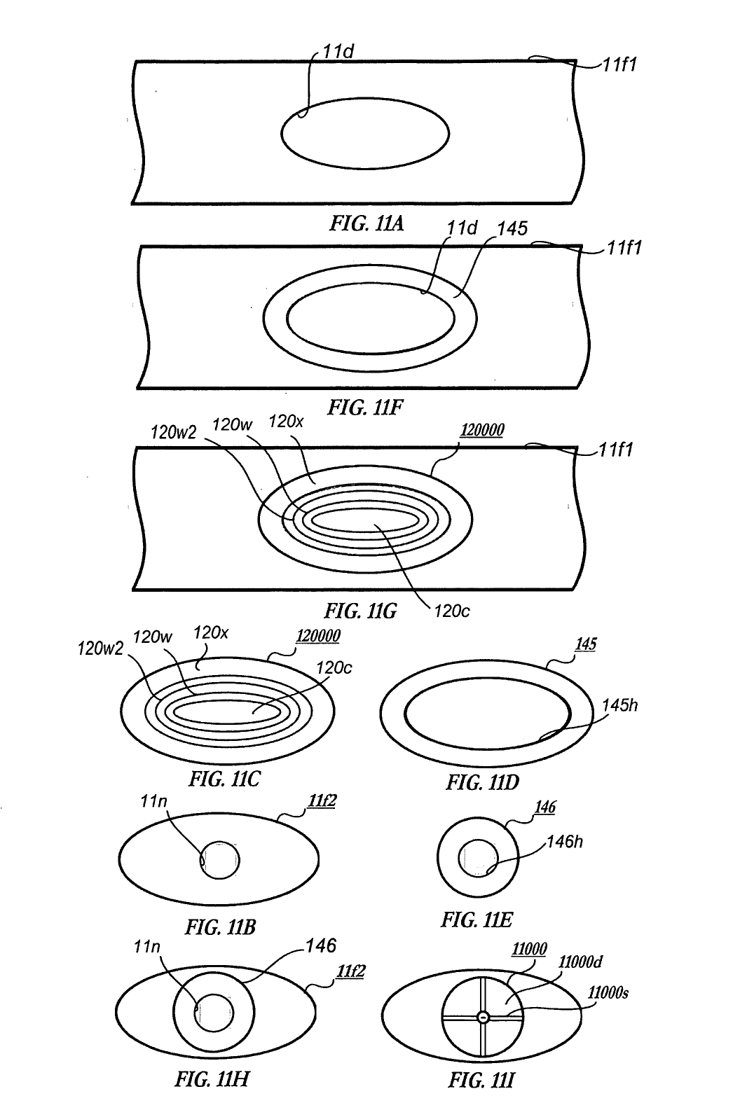

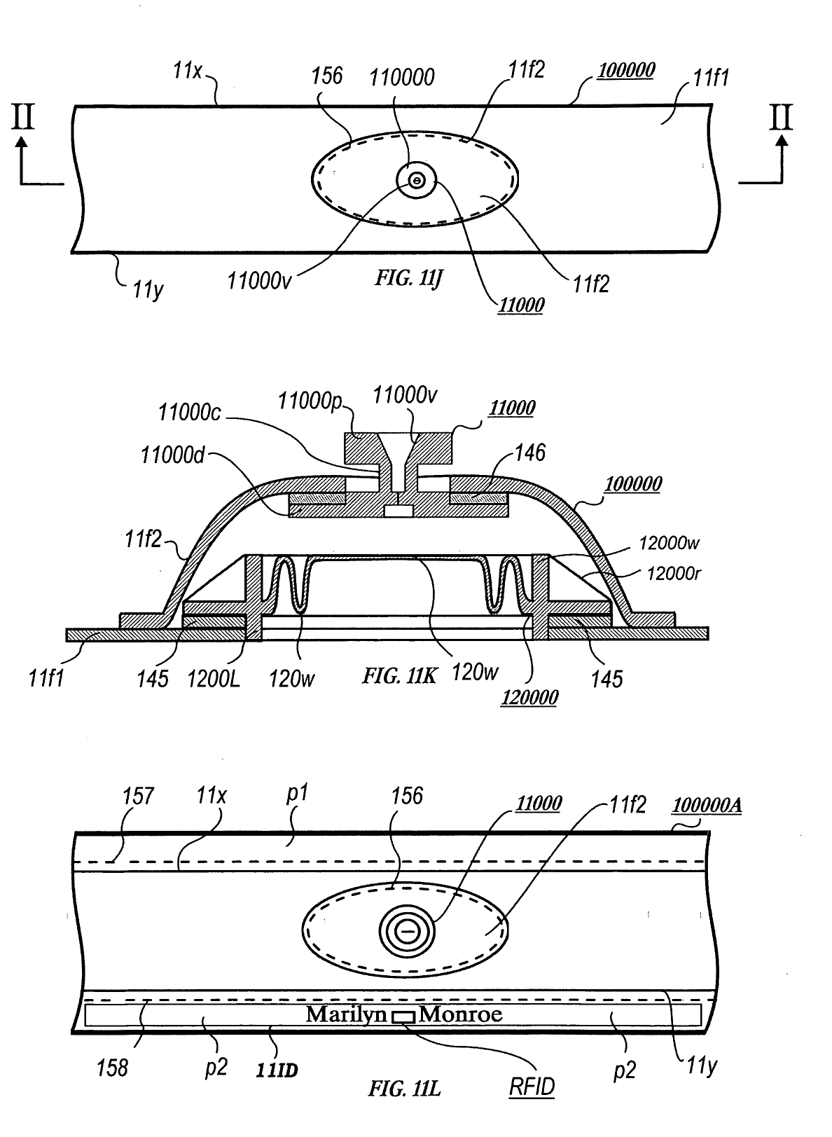

FIG. 10A-10D show a top view, a bottom view and cross sectional views at lines II-II and lines III-III of FIG. 10A according to another embodiment of a diaphragm 12000 according to the present invention. The diaphragm 12000 is similar to the diaphragm 12000 and only the differences therebetween will be described herebelow.

Numeral 12000r designates a plurality of ribs, each of which extends from the outer side of the wall 12000w in a perpendicular direction to the wall 12000w outwardly towards the edge of the diaphragm 12000. The height of the ribs 12000r decreases from the same height as the wall 12000w at the point each of the ribs 12000r adjoins the wall to 0 height along the periphery of the diaphragm 12000.

The wall 12000w and ribs 12000r substantially restrict the axial and radial movement of the outer portion 120x of the diaphragm 12000 outwardly of the wall 12000w when the air bag in which the diaphragm 12000 is mounted in is being inflated with air. However, the central part 120c and the waves 120w and 120w2 are free to move outwardly towards the radial artery. This will provide extra support around the hole of the air bag in which the diaphragm is mounted in. For example, in the case of the air bag 10, the hole 10d in the film 10f, provided for the diaphragm 12000, will have less radial and axial stresses exerted on it (i.e. due to the diaphragm 12000 expanding outwardly and increasing in size) as the air bag 10 is inflated with air.

Numeral 12000L designates an elliptical upwardly facing lip which is integrally formed with the diaphragm 12000. The lip 12000L is integrally formed with the diaphragm 12000 on the opposite side of the wall 12000w and lies just above the wall 12000w (i.e. on the outwardly facing surface of the diaphragm 12000). This lip 12000w serves two purposes;

1. To further block (i.e. act as a dam to prevent the central portion 120c and the waves 120w and 120w2 from moving horizontally along the persons arm; and

2. To prevent the edges of the film 10f around the lip 12000L from coming into contact with the patients skin and thereby preventing skin irritation due to the sharp edge of the film 10f.

Preferably, a small amount of talcum powder should be inserted into the air bag 10 through the nipple 11, to further ensure the proper folding of the diaphragm 12, 120,1200 and 12000, when the respective air bags are deflated.

Air Bag 1 Layer Non Stretchable with Diaphragm

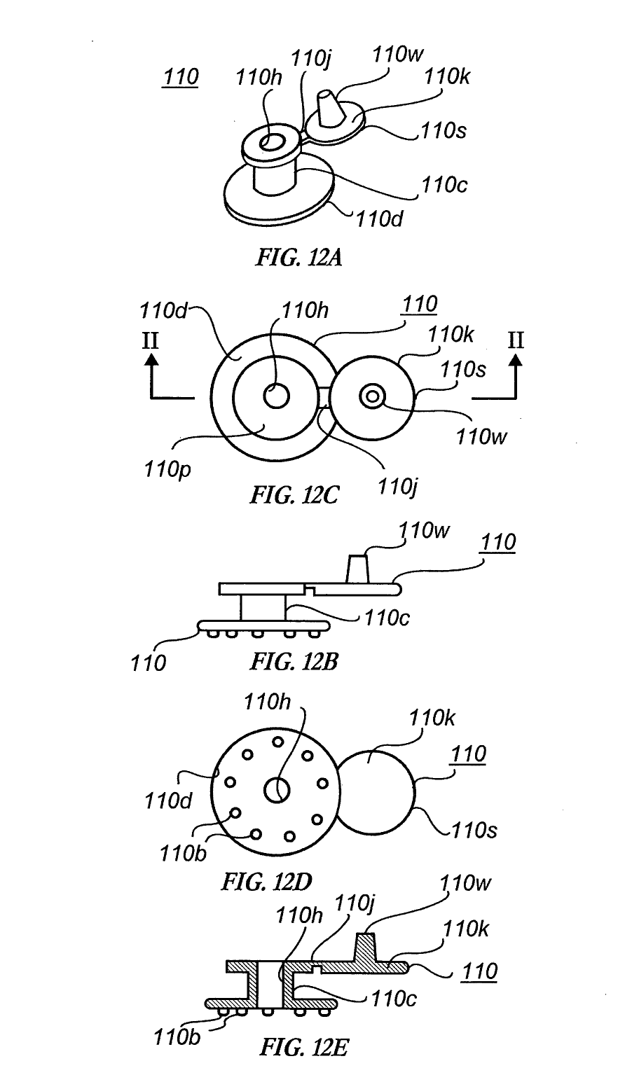

FIGS. 11A-11K show the parts and the manufacturing steps required to manufacture a disposable air bag 100000 according to another embodiment of the present invention.

FIG. 11A shows a top view of a first thin film 11f1. The film 11f1 is long enough to go around a persons hand (i.e. about 30 cm. long) and is about 3 to 5 cm. wide. The film 11f1 is made of thin preferably, clear film and is easily bendable but not stretchable. Numeral 11d designates an oval shaped hole formed along a central portion of the film 11f1, using a punch or a cutting tool.

The oval shaped hole 11h1 is about 5 cm long and 2 to 3 cm. wide and is provided for accommodating the rings 120w, 120w2 and the central portion 120c of the diaphragm 120000 therein.

FIG. 11B shows a top view of a second thin film 11f2. The film 11f2 is oval in shape and has a central hole 11n formed through the center thereof for accommodating the nipple 11000 therein. The film 11f2 is slightly bigger than the hole 11d in the film 11f1.

FIGS. 11D and 11E show top views of double sided tapes 145 and 146.

The DST 145 has an oval shaped hole 145h cut out or punched out of the central portion thereof. The hole 145h in the DST 145 and the hole 11d in the film 11f1 are identical in size and shape. The outer diameter of the oval shaped DST 145 has the same size and shape as the outer diameter of the diaphragm 120000.

The DST 146 has a round shaped hole 146h cut out or punched out of the central portion thereof. The hole 146h in the DST 146 and the hole 11n in the film 11f2 are identical in size and shape. The outer diameter of the round shaped DST 146 has the same outer diameter as the outer diameter of the base portion 11000d of the nipple 11000.

FIG. 11C shows a top view of the diaphragm 120000 described above (shown in FIGS. 10A-10D).

To manufacture the air bag 100000, first the film 11f1 has an elliptically shaped diaphragm hole 11d punched or cut out of the film 11f1 along a central portion thereof. Next, the film 11f2 has a round hole 11n punched or cut out of the central portion thereof for accommodating the nipple 11000 therein. Next, as shown in FIG. 11F, one side of a DST 145 is bonded to the film 11f1 with the hole 11d and 145h aligned with each other. Next, as shown in FIG. 11H one side of a DST 146 is bonded to the film 11f2 with the holes 11n, 146h aligned with each other. Next, as shown in FIG. 11G, the outer portion 120x of the diaphragm 120000 is bonded to the other side of the DST 145. Next, as shown in FIG. 11I, the head portion 11000p of the nipple 11000 is passed through the hole 11n and then the base portion 11000d is bonded to the other side of the DST 146. Next, as shown in FIG. 11J, the film 11f2 is placed on top of the film 11f1 with the nipple 11000 being positioned over the center of the diaphragm 120000 and with the head portion 11000p of the nipple 11000 facing outwards. Next, the films 11f2 is heat sealed (i.e. shown by dash lines 156)

to the film 11f1 along the periphery of the film 11f2 using conventional heat sealing techniques

The width of the films 11f1, 11f2 should be around 30 mm., which is a width comfortable for a person to wear around their wrist. The length of the film 11f1 should be long enough to go around a persons' wrist, i.e. about 30 cm. The length of the film 11f2 should be around 5 cm., i.e. long enough to fit over the radial artery and the surrounding area.

The films 11f1 and 11f2 are made of bendable but not stretchable material such as polyethylene.

It should be noted that instead of the DST 145, the diaphragm 120000 and the film 11f1 may be bonded to each other using glue, heat sealing, ultra sound microwaves, or any other conventional bonding techniques. Similarly, instead of the DST146, the nipple 1100 and the film 11f2 may be bonded to each other using glue, heat sealing, or any other conventional bonding techniques.

The ends of the film 11f1 can have any of the above joining means mounted or attached thereto. For example, male and female Velcro parts may be joined to the ends of the film 11f1 using double sided tape. Alternatively, the strips 1000n and 1000p may be attached to respective ends of the film 11f1 using conventional heat sealing techniques. Or, the disposable air bag 10000 can be wound around a patients' wrist and then the ends of the film 11f1 can be heat sealed to each other at a point where the air bag 10000 loosely fits around the patients writs loosely, but not too loose so that it cannot be pull off the hand of a patient.

FIG. 11L shows a top view of a disposable air bag 100000A according to another embodiment of the present invention.

The air bag 100000A is substantially the same as the air bag 100000 and only the differences therebetween will be described herebelow.

The film 11f1 is cut a little wider (about 40 mm wider) than the film 11f2. After the heat sealing step (shown by dash lines 156) described above, the side edges 11x and 11y of the film 11f1 are folded over and then heat sealed along dash lines 157, 158 to form two pockets p1, p2 along both sides of the air bag 100000A. These pockets p1, p2 can be used to store patient information. One such example is a piece of paper having the name Marilyn Monroe printed on it slid into the pocket p2. Other I.D. devices may include a bar code or a radio frequency I.D. (RFID such as disclosed in U.S. Pat. No. 7,042,346 entitled passive device having information stored therein the subject matter of which is incorporated herewith) device inserted into the pocket p1 and/or p2.

Accordingly, the disposable air bags 100000, 100000A provide a simple, light, comfortable, cheap, extremely high resolution, not painful, irritable or uncomfortable when inflated solution to a needy problem. Furthermore, the air bag 100000,100000A doubles up as a name ID bracelet for patients in hospitals. The air bag 100000, 100000A can be mass produced for less than 1 cent each and accordingly would be an attractive solution for hospitals in third world countries.

Nipple 110

FIG. 12A-12E show a perspective view, a side view, a top view, a bottom view, and a cross sectional view at line II-II of FIG. 12C of an air valve 110 (hereinafter referred to as a nipple 110) according to another embodiment of the present invention. Referring to the Figs., the nipple 110 comprises a cylindrical shaft portion 110c having a radially extending disc like base portion 110d (hereinafter referred to as the base portion 110d) integrally formed with said shaft portion 110c along one end thereof and a radially extending round protrusion 110p (hereinafter referred to as the head portion 110p) integrally formed with said shaft portion 110c along the other end thereof. The nipple 110 has a through hole 110h formed through the center thereof (i.e. through the center of the head portion 110p, through the shaft portion 110c and through the base portion 110d for allowing air to pass therethrough. The nipple 110 further comprises a sealing portion 110s integrally formed therewith for sealing the air bag 100, so as to prevent water from entering into the bag during bathing, or washing hands, etc. The sealing portion 110s comprises a narrow flexible strip 110j, one end of which is integrally formed along an outer edge of the head portion 110p and the other end of which has a round cap portion 110k having a round shaft 110w formed at the center thereof, the round shaft 110w being cone shaped and having an outer diameter at the base thereof which is slightly larger than the inner diameter of the through hole 110h, so that the shaft 110w snugly fits inside the hole 110h when the air bag to which the nipple is coupled to is not being used (i.e. the shaft 110w frictionally fits inside the hole 110h in the head 110p, so as to formed a hermetic water seal therebetween). The base 110d has a larger outer diameter than the head 110p, so that the hole in the air bag in which the head 110p fits through can be smaller than the base portion 110d (i.e. the head portion 110p can fit through the hole provided in the air bag being used while the base portion 110d cannot), so that the nipple 110d can be joined to the air bag by either heat sealing the periphery of the base portion 110d or by using double sided tape.

The nipple 110 may be formed of any flexible plastic material such as polypropylene, nylon, polyethylene, silicon rubber, etc.

Nipple 1100

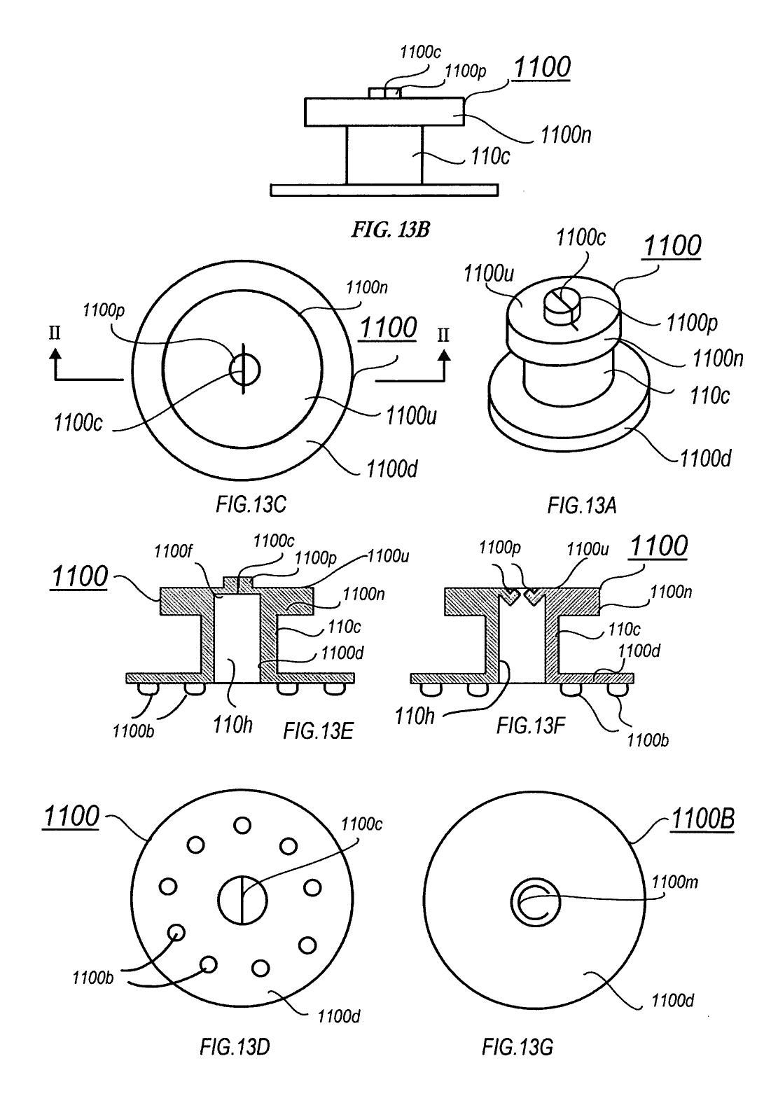

FIG. 13A-13E shows a side view, a top view, a bottom view, a side cross sectional view at line II-II in FIG. 13C of an air valve 1100 (hereinafter referred to as nipple 1100) according to another embodiment of the present invention. The nipple 1100 is similar to the nipple 110 described above and only the differences therebetween will be described herebelow. Referring to the Figs., numeral 1100f designates a flap portion integrally formed at the outwardly facing cylinder end 110c (i.e. in the head portion 1100n) in the radial direction thereof using conventional injection molding techniques. Numeral 1100p designates a round protrusion formed on the outwardly facing surface of the flap 1100f along a central portion thereof. The outer diameter of the round protrusion 1100p is less than the inner diameter of the hole 110h in the cylinder 110c. Numeral 1100c designates a straight cut which is formed using a knife or cutter. The cut 1100c is formed after the injection molding step of the nipple 1100. The cut 1100c is made directly through the center of the protrusion 1100p in the axial direction of the nipple 1100 and the length of the cut is the same as the inner diameter of the hole 110h. Accordingly, the nipple 1100 is normally sealed (i.e. closed position) by the flap 1100f and thereby prevents any water from getting into the air bag in which the nipple 1100 is mounted in. However, when pressure is applied downwards on the protrusion 1100p (i.e. as when a clip 130 shown in FIG. 16 is mounted on the nipple 1100), the protrusion 1100p is pressed downwards causing the flap 1100f to partially open (i.e. as shown in FIG. 13F), thereby allowing air to pass through the nipple 1100, while providing a hermetic seal between the upper surface 1100u of the head portion 1100n of the nipple 1100 and the lower surface 13L of the upper arm 13U of the clip 130 (shown in FIG. 16). After the force on the nipple 1100 is removed (i.e. the clip 130 is removed), the protrusion 1100p returns to its original position, due to the resilient nature of the material of which the nipple 1100 is made of.

FIG. 13G shows a bottom view of the nipple 1100B according to another embodiment of the present invention. The nipple 1100B is substantially the same as the nipple 1100 and the only difference therebetween is that instead of the straight cut 1100c the cut is semicircular 1100m. The cut 1100m has a diameter which is the same as or smaller than the inner diameter of the cylinder 1100d but larger than the protrusion 1100p. The semi-circular cut 1100m is made using a sharp punch having a blade in the shape of a semi-circle. The cut 1100m is formed after the injection step of the nipple 1100. A round punch (not shown) has about one or two mm. of its cutting edge filed of, so that when the punch is used to cut the cut 1100m, it leaves the flap 1100f partially attached to the nipple 1100 along a small portion thereof, whereby the attached portion acts as a spring, due to the resilient properties of the material used to form the nipple 1100, to normally keep the nipple 1100 in a closed position, thereby preventing water from entering into the bag to which the nipple 1100 is attached to.

When the clip 130 is mounted on the nipple 1100, the lower surface 13L of the upper arm 13 presses down on the protrusion 1100p causing the protrusion 1100p to deform downwardly and to open the air valve 1100 to allow air to pass therethrough. The outer diameter of the round protrusion 1100p is smaller than the inner diameter 110h of cylinder 110c, so that pressing down on the protrusion 1100p causes the cap (and 1100f) to move downwards into the cylinder 110c and to open the nipple 1100 to allow air to pass therethough.

At the same time, since the bottom surface 13L of the arm 13U of the clip 130 presses down on the top surface 1100u of the head portion 1100n of the nipple 1100, the clip 130 and the nipple 1100 form a hermetic seal therebetween. Accordingly, pressurized air can pass through the clip 130 and into the nipple 1100 without having any air leak between them (i.e. at the point where the lip 1100p contacts the bottom surface 13L of the clip 130.

Nipple 11000

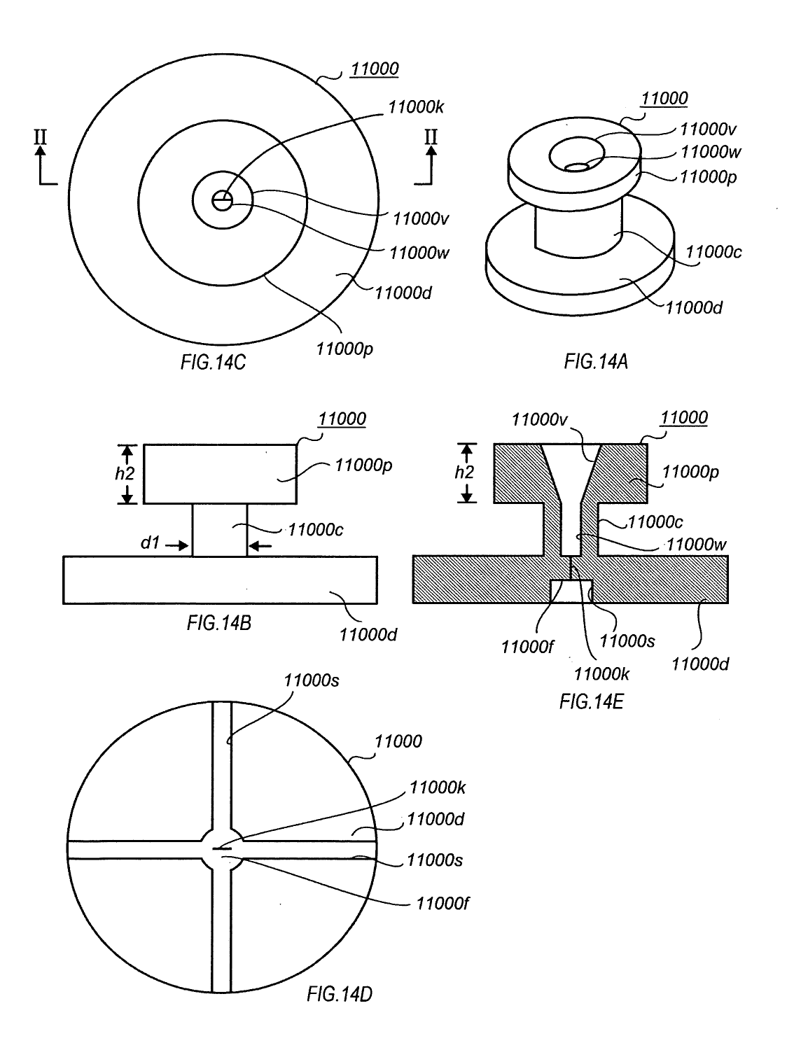

FIG. 14A-14E show a perspective view, a side view, a top view, a bottom view and a side cross sectional view at line II-II in FIG. 14C of a nipple 11000 according to another embodiment of the present invention. Referring to the Figs., numeral 11000c designates a round shaft, numeral 11000d designates a round disc like base portion (hereinafter referred to as base portion 11000d or base 11000d) integrally formed with the shaft 11000c along a bottom end thereof, numeral 11000p designates a round head portion (hereinafter referred to as head portion 11000p or head 11000p) integrally formed with the shaft portion 11000c along the other end thereof. Numeral 11000v designates a cone shaped cavity formed through the center of the head portion 11000p. The cone shaped cavity 11000v extends from the top surface of the head portion 11000p to a central point therein. Numeral 11000w designates a round cylindrical hole which extends through the center of the shaft portion 11000c from the inner end of the cone shaped cavity 11000v to a central point of the base portion 11000d. Numeral 11000s designate two diagonal slots formed along the bottom surface of the base portion 11000d. Numeral 11000f designates a round flap integrally formed with the nipple 11000 and extends from the inner end of the hole 1000w to the top of the slot 11000s. The nipple 11000 is formed from flexible rubber, nylon, enca vynil, polypropylene, silicone or any other suitable flexible material using conventional injection molding techniques.

Numeral 11000k designates a straight cut made through the center of the flap 11000f by a sharp object like a knife or punch. The length of the cut 1000k is the same as the inner diameter of the hole 11000w. Normally, the flap 11000f blocks water from flowing through the nipple 11000, namely, through the cone shaped cavity 11000v and the hole 11000w to the slots 11000s in the nipple 11000.

Clip 13

FIG. 15A-15E show a perspective view, a side view, a top view, a bottom view, and a side cross sectional view at line II-II of FIG. 15C of a connector means 13 (hereinafter referred to as air valve connector 13 or clip 13) for hermetically connectively/disconnecting an air hose 14 to the nipple 11 or nipple 111 (shown in FIGS. 3 and 4) according to the present invention. FIG. 15F shows a side view of the clip 13 having an air hose 14 connected thereto.

Referring to FIGS. 15A-15F, the clip 13 comprises an upper rectangular arm portion 13U, a lower rectangular arm portion 13L and a rectangular bar portion 13B, the respective ends of the bar portion 13B being integrally formed with said arm portions 13U and 13L along central portions of said arm portions. The clip 13 is formed of a plastic which is flexible such as acryl, polypropylene, etc and preferable in made of a clear plastic using conventional injection molding techniques. Accordingly, when the back ends 13D and 13E are manually pressed toward each other, the front ends 13A and 13B move away from each other and when the clip is released, the front ends 13A, 13B and back ends 13D, 13E return to their original positions, due to the elastic nature of the clip 13. Numeral 13c designates a cylindrical portion integrally formed with the clip 13. The cylindrical portion 13c is formed on the top surface 13u of the upper arm 13U along the center of the front part of the upper arm 13U and is perpendicular to the top surface 13u of the upper arm portion 13U. Numeral 13v designates a cone shaped protrusion integrally formed with the cylindrical portion 13c along a central part thereof. Numeral 13h designates a through hole formed inside the cylinder 13c and extends all the way to the bottom surface 13y of the upper arm 13U.

Numeral 14 designates a silicon tube (or any other suitable flexible material) one end of which is frictionally mounted on the cylinder 13h. The hose 14 is prevented form slipping off the cylinder 13c by the protrusion 13v. Numeral 13d designates a hole formed through the center of the upper arm 13U. The hole 13d is used for allowing the air hose 14 to pass therethrough, so that the hose 14 does not interfere with the manual operation of the clip 13.

To use the clip 13 with the air bag 10, the back ends 13D, 13E of the clip 13 are pressed towards each other, causing the front ends 13A and 13B to move away from each other. Next, the air bag 10 is slid into the space between the front ends 13A, 13B until the hole 11h in the nipple 11 is aligned with the hole 13h in the clip 13 and then the clip 13 is released causing the clip 13 to clamp down on the air bag 10, thereby allowing air to flow through the hose 14, the clip 13 the nipple 11 and into and out of the air bag 10, while maintaining a hermetic seal between the clip 13 and the nipple 11. Namely, the pressure provided by the clip 13 on the nipple 11 causes the top and bottom surfaces 13y, 13x of the arms 13U and 13L to press the bottom surface 13y of the upper arm 13U against the top surface of the nipple 11 to form a hermetic seal therebetween.

For the case where the nipple 111 is used instead of the nipple 11 in the air bag 10, the bottom surface 13y of the upper arm 13U of the clip 13 would press down on the protrusion 111p and cause the nipple 111 to open and allow air to pass therethrough.

It should be noted that the hole 13h in the clip 13 should be smaller than the outer diameter of the protrusion 111p in the nipple 111 for the clip 13 to effectively press down on the protrusion 111p and open the nipple 111 to allow air flow therethrough.

Clip 130

FIGS. 16A-16E show a perspective view, a side view, a top view, a bottom view and a cross sectional view at line II-II of FIG. 16C of a clip 130 according to another embodiment of the present invention. FIG. 16F shows a side view of the clip 130 having a hose 14 connected thereto.

The clip 13 and 130 are very similar to each other and only the differences therebetween will be described herebelow.

Referring to FIGS. 16A-16F, the bottom arm 13L has a slot 130s formed therein. The slot 130s extends from a front end 13B of said lower arm portion 13, the width of said slot being the same as the outer diameter of said shaft portion of said nipple, and the length of said slot being substantially the same as the size of the outer diameter of said heat portion of said nipple, whereby said shaft portion of said nipple can slide into said slot in said lower arm portion and said head portion of said nipple can be clamped between said upper and lower arm portions to form a hermetic seal therebetween.

More specifically, the slot 130s extends from the front end 13B of the bottom arm to a point 130e which is partially past the bottom of the hole 13h in the upper arm 13U. The width W of the slot 130s is the same as or slightly bigger than the diameter of the shafts 110c of the nipples 110, 1100 (or the shaft 11000c of the nipple 11000), so that the nipples shafts 110c can slide into the slot 130s in the clip 130. When the shaft 110c is slid all the way into the slot 130s until the shaft 110c buts up against the back end 130e of the slot 130s, the holes 13h in the upper arm 13U and the hole 110h in the nipple 110, or the hole 110h in the nipple 1100 are lined up so that pressurized air in the hose 14 can flow through the nipple 110 or 1100 and into the air bag in which these nipples 110, 1100 are mounted in. The hole 13h in the top arm 13U of the clip 130 is smaller than the round protrusion 1100p in the nipple 1100, so that when the clip 130 is mounted on the nipple 1100, the bottom surface 13y of the top arm 13U pushes the protrusion 1100p into the hole 110h in the nipple 1100 (i.e. as shown in FIG. 13F) to open the nipple 1100 to allow air to flow therethrough.

Clip 1300

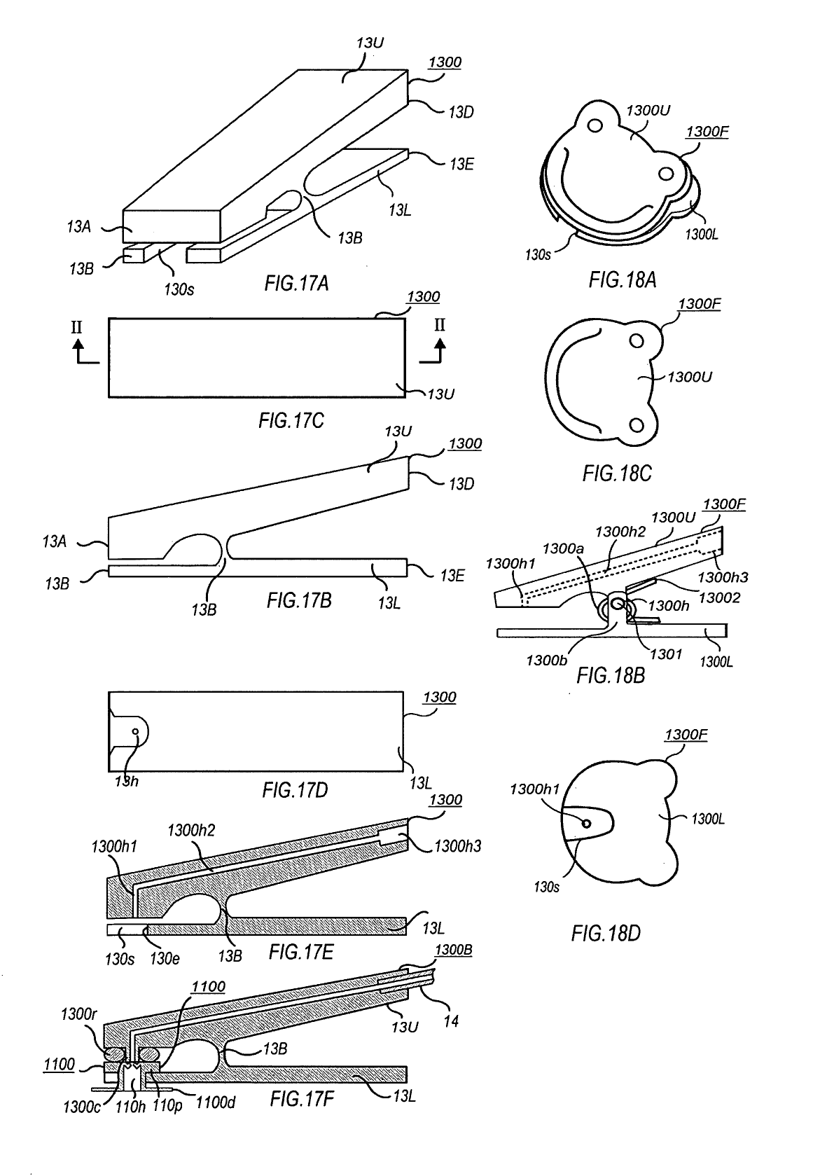

FIGS. 17A-17E show a perspective view, a side view, a top view, a bottom view, and a side cross sectional view at line II-II of FIG. 17C of a clip 1300 according to another embodiment of the present invention. The clip 1300 is similar to the clip 130 and, accordingly, only the differences therebetween will be described herebelow. Referring to the Figs., it can be seen that the cylinder 13c and the hole 13h of the clip 130 are not used in the clip 1300. Instead of the cylinder 13c and the hole 13h, the upper arm 13U comprises a vertical hole 1300h1 and horizontal holes 1300h2h, 1300h3 which pass through the inside of the upper arm portion 13U from the front, bottom surface 13y of the upper arm 13U to the back end 13D of the upper arm portion 130U. The diameter of the horizontal hole 1300h3, near the back end 13D, is enlarged to allow one end of the hose 14 to frictionally fit therein.

Numeral 130s designates a slot formed in the lower arm portion 13L. The slot 130s extends from the front end 130B of the bottom arm portion 13L to a point which is just past the hole 1300h1. The width of the slot 130s should be the same as the outer diameter of the cylindrical portion 110c of the nipple 1100, so that the cylindrical portion 110c can slide into the slot 130s until the cylinder portion 110c buts up against the end of the end 130e of the slot 130s at which time the hole 1300h1 should be lined up with the hole 110h in the nipple 1100 (i.e. the hole 1300h1 of the clip 1300 should be right on top of the protrusion 100c of the nipple 1100). The hole 1300h1 should be smaller than the outer diameter of the protrusion 1100p, so that the protrusion 1100p can be pushed down by the upper arm 13U when the clip 1300 is mounted on the nipple 1100.

FIG. 17F shows a side cross sectional view of a clip 1300B according to another embodiment of the present invention. In the Fig., the clip 1300B is mounted on the nipple 1100.

The clip 1300B is the similar to the clip 1300 and only the differences therebetween will be described herebelow.

Referring to FIG. 17F numeral 1300c designates a cylindrical portion integrally formed along the bottom surface 13y of the upper arm 13U. The central hole in the cylindrical portion 1300c and the hole 1300h1 in the upper arm 13U have the same diameters and are aligned with each other. The outer diameter of the cylindrical portion 1300c is the same as the outer diameter of the protrusion 1100p in the nipple 1100. Numeral 1300r designates a round rubber O ring mounted on the cylindrical portion 1300c. The O ring 1300r provides a better air seal between the clip 1300B and the top surface of the nipple 1100.

Face Clip 1300F

FIG. 18A-18D show another embodiment of a clip 1300F according to another embodiment of the present invention. Referring to the Figs., numeral 1300U designates an upper arm, numeral 1300L designates a lower arm, numerals 1300a and 1300b each designate a pair of legs integrally formed on the lower and upper surfaces of the upper and lower arms 1300U and 1300L, respectively. Each of the legs 1300a and 1300b have a round hole 1300h formed therein. The holes are aligned with each other and a pin 1301 is inserted therein, thereby locking the upper arm 1300U and the lower arm 1300L with each other. Accordingly, the upper arm 1300U and the lower arm 1300L can partially swivel with respect to each other.

To assemble the clip 1300F, first the holes 1300h in the legs 1300a, and 1300b of the upper arm 1300U and the lower arm 1300L are aligned with each other. Next the middle portion of a round spring 13002 is placed between the legs 1300a and 1300b and then the pin 13001 is inserted into the holes 1300h in the legs 1300a and 1300b. The respective ends of the spring but up against the inner surfaces of the upper arm 1300U and the lower arm 1300L. Accordingly, the spring 1301 keeps a constant force on the front leg portions 1300U and 1300L. To open the clip 1300 pressure must be applied to the back end of the clips 1300U and 1300L. This structure is commonly used in the art of clips.

The upper arm portion 1300U and lower arm portion 1300L are each shaped in the shape of a happy face, so that when this clip is mounted on a nipple of an air bag which is on a patient, the visual effect on the patient will have a relaxing and happy effect on the patient about to have their blood pressure measured.

The upper arm 1300U has the holes 1300h1, 1300h2 and 1300h3 formed therein similar to the upper arm 13U of the clip 1300. The air hose 14 can be mounted in the hole 1300h3. The lower arm 1300L has the slot 130s formed therein. Accordingly, the clip 1300F (hereinafter referred to as the happy face clip 1300U) is the same in function as the clip 1300.

One major object of providing happy face clip is to bring a smile to the child or adult having their blood pressure measured, so that the measured blood pressure will be more accurate, since the patient stays relaxed and does not tense up. The whole operation of measuring the blood pressure of a patient using the happy face clip with any of the air bags 10, 100, 1000, etc., should be a comfortable fast and happy experience, thereby providing a more accurate blood pressure measurement.

The present invention is not limited to the shape of this particular happy face clip and any Walt Disney character or other character can be used without departing from the spirit of the present invention.

Clip 13000

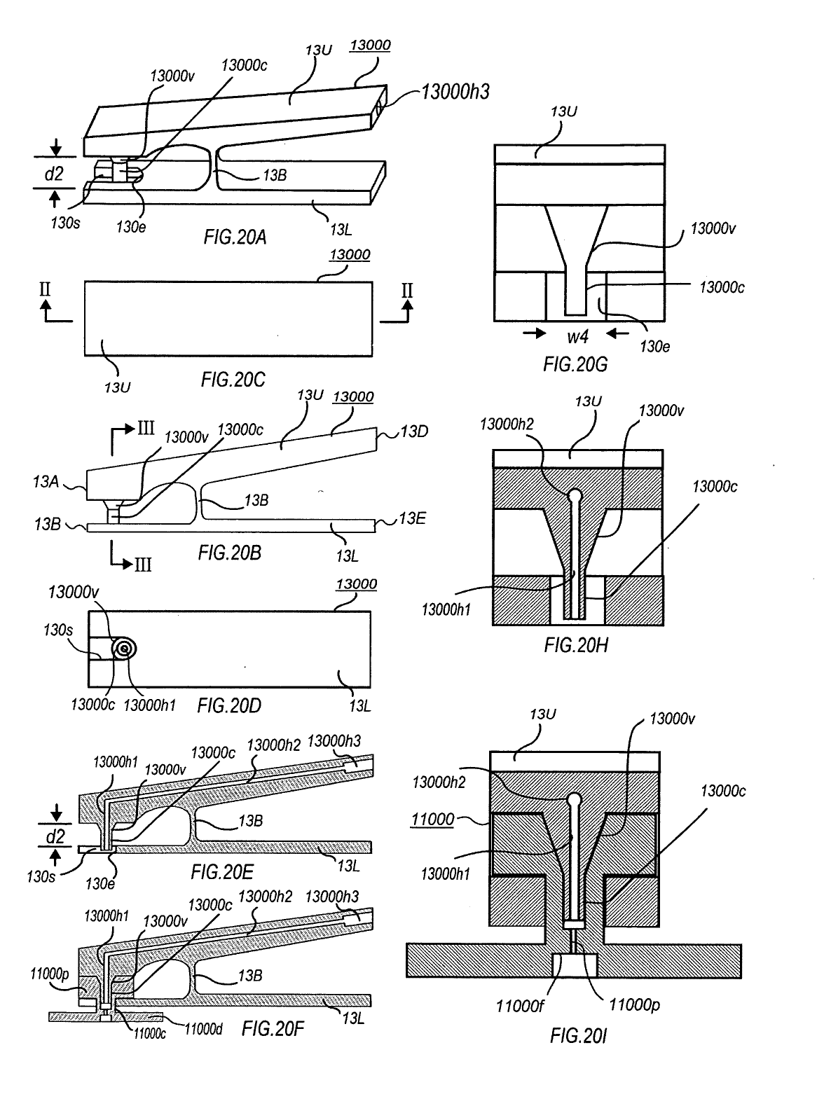

FIGS. 20A-20E show a perspective view, a side view, a top view, a bottom view, and a cross sectional view at line II-II in FIG. 20C of the clip 13000 according to the present invention. FIG. 20F shows a cross sectional view at line II-II in FIG. 20C of the clip 13000 with the nipple 11000 mounted therein. FIG. 20G-20H show and end view and an end cross sectional view at line III-III in FIG. 20B of the clip 13000. FIG. 20I shows an end cross sectional view at line III-III in FIG. 20B of the clip 13000 with the nipple 11000 mounted therein.

Referring to FIGS. 20A-20I, numeral 13U designates an upper rectangular shaped arm, numeral 13L designates a lower rectangular shaped arm, and numeral 13B designates a rectangular shaped leg. The respective ends of the leg 13B are integrally formed with the bottom side of the upper arm 13U along a central portion of the upper arm 13U and the top side of the lower arm 13L along a central portion of the lower arm 13L. The width w1 of the rectangular leg 13B is much thinner than the width w2, w3 of the upper and lower arms 13U, 13L, so that when the back ends 13D, 13000y are manually pressed towards each other, the front ends 13A, 13B of the arms 13U, 13L move away from each other due to the flexible nature of the leg 13B. Furthermore, when the back ends 13D, 13E of the arms 13U, 13L are released, the arms 13U and 13L return to their original closed position, due to the flexible nature of the leg 13B. Since the arms 13U and 13L are wider (i.e. thicker) than the leg portion 13B, the arms portions 13U, 13L are much stiffer then the leg 13B and, accordingly, bend very little when the back ends 13D, 13E of the arms 13U, 13L are manually pressed towards each other.

Numeral 130s designates a rectangular slot formed in the front bottom arm 13L. The slot 130s extends from the front portion of the lower arm 13L to a central point in the front portion of the bottom arm 13L.

Numeral 13000v designates a cone shaped protrusion integrally formed along the bottom central front portion of the upper arm 13U and numeral 13000c designates a round shaft one end of which is integrally formed with the extending end of the cone shaped protrusion 13000v.

The cone shaped protrusion 13000v and the shaft 13000c are perpendicular to the lower surface of the upper arm 13U and the extending end of the round shaft 13000c extends to a point central to the slot 130s formed in the front portion of the bottom arm 13L.

Numeral 13000h2 designates a horizontal hole formed in the center of the upper arm 13U. The hole 13000h2 extends from the back end of the upper arm 13U to a central point of the front portion of the upper arm 13U. Numeral 13000h1 designates a vertical hole formed in the upper arm 13U, the center of the cone shaped portion 13000v and the shaft portion 13000c. The horizontal and vertical holes 13000h2 and 13000h1 are connected to each other inside the upper arm portion 13U. The diameter of the horizontal hole 13000h3 at the back end of the upper arm 13U is slightly bigger, so that it can accommodate one end of a flexible air hose 14, made of silicone, nylon, or any other suitable material, therein.

The length of the cone shaped protrusion 13000v and the shaft 13000c of the clip 13000 are the same as the respective length's of the cone shaped cavity 11000v and round hole 11000w formed in the nipple 11000.

The width w4 of the slot 130s in the front portion 13B of the bottom arm 13L is the same as or slightly bigger than the outer diameter d1 of the shaft 11000c of the nipple 11000, so that the shaft portion 11000c of the nipple 11000 can easily slide through the slot 130s in the clip 13000.

Furthermore, in the normal state of the clip 13000 (i.e. when no pressure is applied to the arms 13D and 13E) the distance d2 between the lower and upper surfaces of the front end arm portions 13U and 13L is slightly less then the height h2 of the round protrusion 11000p, so that the back ends 13D, 13E of the legs 13U, 13L need only be moved a relatively small amount in order to be able to slide the round shaft 11000c and the head portion 11000p between the upper and lower leg portions 13U and 13L, thereby allowing for easy mounting of the nipple 11000 in the clip 13000, while still providing sufficient pressure between the lower surface of the arm 13U and the upper surface of the round protrusion 11000p, to prevent any air escaping therebetween. At this time (i.e. when the round shaft 13000c and cone shaped protrusion 13000v are respectively inside round hole 11000w and the cone shaped cavity 11000v) the shaft portion 11000c of the nipple 11000w is adjacent to the end of the slot 130s in the clip 13000. Accordingly, with this clip 13000 and nipple 11000, very easy alignment can be achieved during the mounting of the clip 13000 on the nipple 11000.

The outer diameter of the cone shaped protrusion 13000v and the round shaft 13000c are slightly bigger than the respective inner diameters of the cone shaped cavity 11000v and round hole 1000w. Accordingly, when the clip 13000 is mounted on the nipple 11000, the round shaft 13000c and cone shaped protrusion 13000v of the clip 13000 penetrate the round shaped hole 11000w and the cone shaped cavity 11000v, respectively, and cause the cone shaped cavity 11000v and the round hole 11000w to slightly spread (i.e. the cone shaped cavity 11000v and round shaft 11000w are slightly stretched), thereby providing a very good air seal therebetween. Furthermore, due to the spreading of the round hole 11000w in the nipple 11000 by the round shaft 13000c of the clip 13000, the flap 11000f spreads apart at the cut 11000x, causing a gap 11000p to open up in the flap 11000f, whereby air passing through the holes 13000h2 and 13000h1 of the clip 13000 is able to pass through the gap 11000p and through the nipple 11000 into the air bag in which the nipple 11000 is mounted in.

Clamp Normally Open

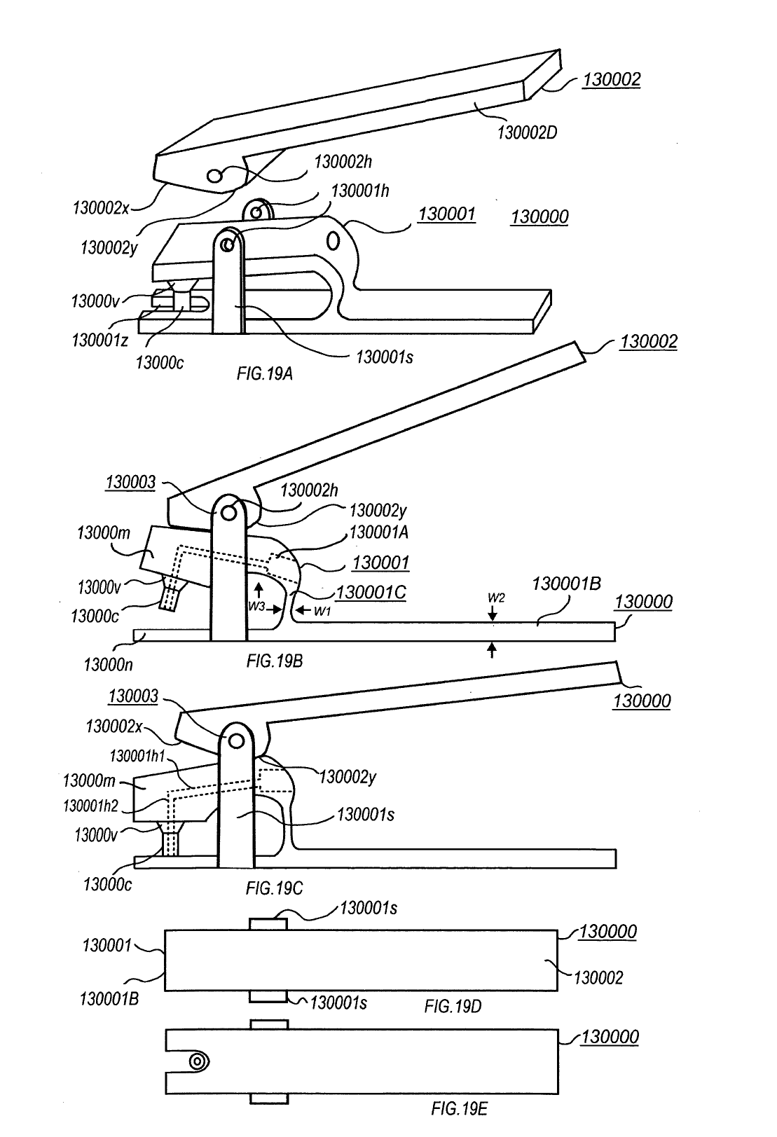

FIG. 19A shows a perspective view of a clamp 130000 according another embodiment of the present invention. FIGS. 19B-19E show a side view with the clamp 130000 in the normally open position, a side view with the clamp 130000 in the closed position, a top view, and a bottom view of the clamp 130000. The clamp 130000 is used as a means for connectively disconnecting an air bag to and from an electronic pressure measuring device according to the present invention.

Referring to FIGS. 19A-19E, numerals 130001, 130002, 130003 generally designate a first, second and third part required to make the clamp 130000 according to the present invention.

First, the first part 130001 of the clamp 130000 will be described. Numeral 130001A designates an upper rectangular shaped arm, numeral 130001B designates a lower rectangular shaped arm, and numeral 130001C designates a rectangular shaped leg. The respective ends of the leg 130001C are integrally formed with the bottom surface of the upper arm 130001A along a central portion of the upper arm 130001A and the top surface of the lower arm 130001B at the back end of the lower arm 130001B.

Numeral 130001s designates a pair of identical side legs, each of the legs 130001 having one end thereof integrally formed with the bottom arm 130001B along respective sides of the bottom arm 130001B at a central portion of the bottom arm with respect to the length thereof. The side supports 130001s extend upwards past the sides of the upper arm 130001A. Numeral 130001h designates a through hole formed in each of the supports 130001s.

Next, the second part of the clamp 130002 will be described herebelow. Numeral 130002 designates a clamp locking arm for locking and opening the clamp 130000. The locking arm 130002 comprises a rectangular shaped arm having a round hole 130002h formed therethrough at one end thereof

The third part 130003 of the clamp 130000 comprises a round shaft for mounting the locking arm 130002 to the first part 130001.

The hole 130002h is formed in the end of the arm 130002 at a point which is off-center with respect to the edges 130002X and 130002Y of the sides of the end of the arm 130002. The edges 130002X, 130002Y are each flat surfaces which provide two stable states, a clamp open state and a clamp locked state, as will be explained more fully herebelow.

To assemble the locking arm 130002 in the first part 130001, the holes 130001h and 130002h are aligned with each other and the round shaft 1300003 is pushed into the holes 130001h and 130002h. The length of the shaft 130003 is set to be the same as the length of the holes 130001h and 130002h when assembled.

The locking arm 130002 is free to partially rotate with respect to the first part 130001.

When the locking handle 130002 is in the unlocked (i.e. up position as shown in FIG. 19B), the surface 130002x of locking handle 130002 is pressing against the top surface of the upper arm 130001A of the first part 130001.

When the locking handle 130002 is turned from the unlocked position described above to the locked position, the locking handle 130002 is swiveled downwards (i.e. manually pushed down) towards the back portion of the top arm 130002A (i.e. using the index and thumb fingers) to cause the surface 130002Y to come into contact with the top surface of the top arm 130002A to cause the front portions 130001A and 130001B to come closer together to lock the nipple in the clamp 130000.

The width w1 of the rectangular leg 130001C is much thinner than the width w2, w3 of the upper and lower arms 130001A, 130001B, so that when the back ends 130001x of the first part 130001 and the back end 130002y of the locking arm 130002 are manually pressed towards each other, the front ends 13A, 13B of the arms 13U, 13L move towards each other due to the flexible nature of the leg 13B.

When the locking handle 130002 is turned from the locked position described above to the unlocked position, the locking handle 130002 is swiveled upwards (i.e. manually pushed up) away from the back portion of the top arm 130002A to cause the surface 130002X to come into contact with the top surface of the top arm 130001A to cause the front portions 130001A and 130001B to move away from each other to unlock the nipple in the clamp 130000, (i.e. due to the flexible nature of the leg 130001C.

Numeral 130001z designates a rectangular slot formed in the front bottom arm 130001B. The slot 130001z extends from the front portion of the lower arm 130001B to a central point in the front portion of the bottom arm 130001B. q

Numeral 130001v designates a cone shaped protrusion integrally formed along the bottom central front portion of the upper arm 130001A and numeral 130001w designates a round shaft one end of which is integrally formed with the extending end of the cone shaped protrusion 130001v.

The cone shaped protrusion 130001v and the shaft 130001w are perpendicular to the lower surface of the upper arm 130001A and the extending end of the round shaft 130001w extends to a point central to the slot 130001z formed in the front portion of the bottom arm 130001B.

Numeral 130001h1 designates a horizontal hole formed in the center of the upper arm 130001A. The hole 130001h1 extends from the back end of the upper arm 130001A to a central point of the front portion of the upper arm 130001A. Numeral 130001h2 designates a vertical hole formed in the upper arm 130001A, the center of the cone shaped portion 130001v and the shaft portion 130001w. The horizontal and vertical holes 130001h1 and 130001h2 are connected to each other inside the upper arm portion 130001A. The diameter of the horizontal hole 130001 at the back end of the upper arm is slightly bigger, so that it can accommodate one end of a flexible air hose made of silicone, nylon, or any other suitable material.

The length's of the cone shaped protrusion 130001v and the shaft 130001w of the clamp 130000 are the same as the respective length's of the cone shaped cavity 11000v and round hole 11000w formed in the nipple 11000.

The width w1 of the slot 130001w is the same or slightly bigger than the outer diameter d1 of the shaft 11000c of the nipple 11000, so that the shaft portion 11000c of the nipple 11000 can easily slide through the slot 130001z in the clamp 130000.

Furthermore, in the open unlocked state of the clamp 130000, the distance d2 between the lower and upper surfaces of the front end arm portions 130001A and 130001B is slightly bigger then the height h2 of the round protrusion 11000p, so that the legs 130001x, 130001y need only be moved a relatively small amount in order to be able to lock and hermetically seal the round shaft 130001w and cone shaped protrusion 130001v into the cone shaped cavity 11000v and the round hole 11000w, thereby allowing for easy mounting of the nipple 11000 in the clamp 130000, while still providing sufficient pressure between the lower surface of the arm 130001A and the upper surface of the round protrusion 11000p, to prevent any air escaping therebetween. At this time (i.e. when the round shaft 130001w and cone shaped protrusion 130001v are inside the cone shaped cavity 11000v and the round hole 1000w) the shaft portion 11000c of the nipple 11000 is adjacent to the end of the slot 130001z in the clamp 130000. Accordingly, with this clamp 130000 and nipple 11000, very easy alignment can be achieved during the mounting of the clamp 130000 on the nipple 11000.

The outer diameter of the cone shaped protrusion 130001v and the round shaft 130001w are slightly bigger than the respective inner diameters of the cone shaped cavity 11000v and round hole 11000w. Accordingly, when the clamp 130000 is mounted on the nipple 11000, the round shaft 130001w and cone shaped protrusion 130001v penetrate the cone shaped cavity 11000v and the round hole 11000w, respectively, and cause the cone shaped cavity 11000v and the round hole 11000w to slightly spread (i.e. the cone shaped cavity 11000v and round shaft 11000w are stretched wider), thereby providing a very good air seal therebetween. Furthermore, due to the spreading of the round hole 11000w in the nipple 11000 by the round shaft 130001w of the clamp 130000, the flap 11000f spreads apart at the cut 11000x, causing a gap 11000p to open up in the flap 11000f, whereby air passing through the holes 130001h1 and 130001h2 is able to pass through the gap 11000p and through the nipple 11000 into the air bag in which the nipple 11000 is mounted in.

Stainless Steel Clip

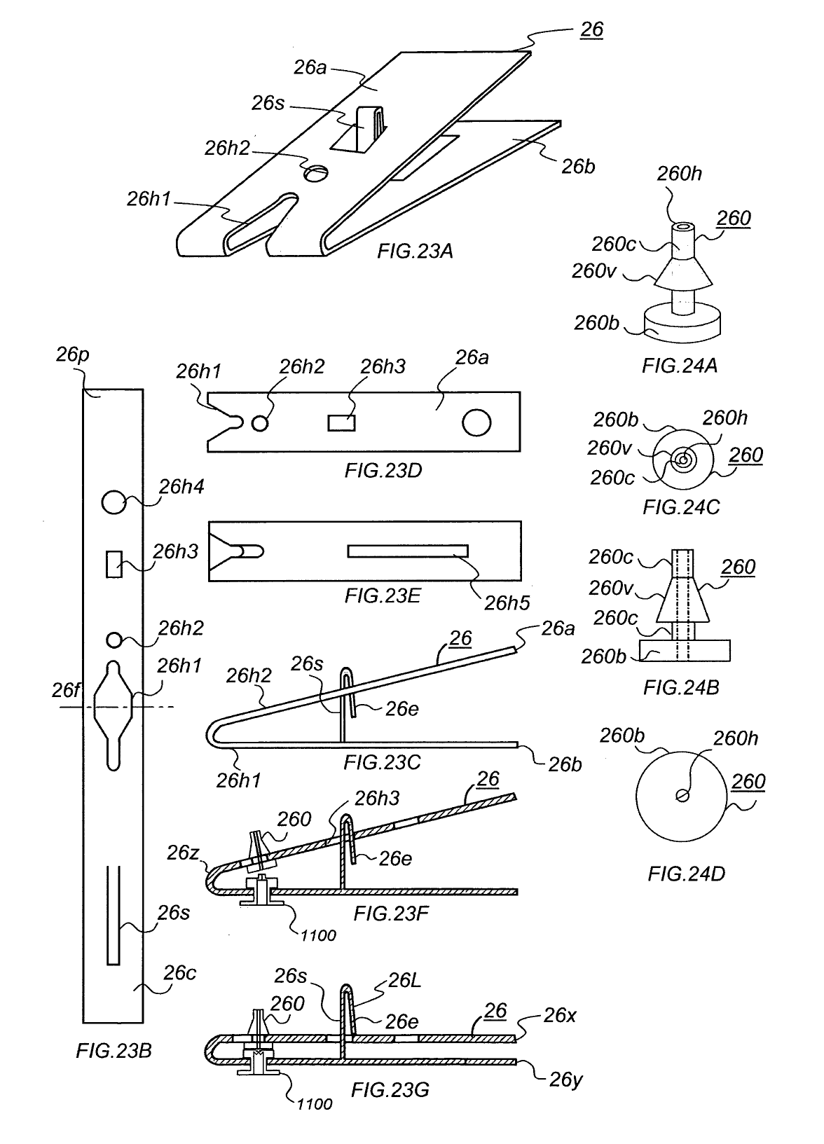

FIG. 23A show a perspective view of a stainless steel clip 26 according to another embodiment of the present invention. FIG. 23B shows a front view of a rectangular piece of sheet steel 26P before being bent into the shape of the clip 26. FIGS. 23C-23E show a side view, a top view and a bottom view of the clip 26. FIGS. 23F-23G show side view of the clip 26 in the open and closed states with an air hose attaching means 150 (i.e. shown in FIG. 24) mounted therein and with the nipple 1100 mounted therein.

Referring to the FIGS. 23A-23I, numeral 26 generally designates the clip made of resilient stainless steel. To manufacture the stainless steel clip 26, first a sheet of stainless steel (not shown) is cut using a pressing machine (not shown) in the shape shown in FIG. 23B The cut piece 26p is substantially rectangular in shape and has apertures h1-h4 punched out of the sheet rectangular strip 26p.

Numeral 26s designates a rectangular strip which is partially cut out of the inside the piece 26p. Next, the rectangular strip 26s is bent upwards by 90 degrees so that it is perpendicular to the rest of the piece 26c. Next, the piece 26p is folded at line 26f by 150 degrees so that the upper and lower folded parts (hereinafter referred to as upper and lower arms 26U and 26L) are at a 30 degree incline with respect to each other. At this time, the extending end of the strip 26s extends through the center of the square aperture 26h3. Next the extending end 26e of the strip 26s is bent 170 degrees backward so that it forms a triangular shaped latch 26L for locking the upper and lower arms 26U and 26L in a parallel position with respect to each other. Namely, when the extending ends 26x, 26y of the arms 26U and 26L are manually pressed towards each other, the latch 26L locks in the edge of the aperture h3 (i.e. the extending end 26e of the strip 26s moves past the side of the aperture h3 and then, once the extending end 26e passes by the edge of the aperture 26h3, the end 26c bounces over the edge of the aperture 26h3. To release the arms 26U and 26L, the latch 26L is pushed forward (i.e. towards the hole 26h1 hereinafter referred to as the front end 26z of the clip 26), whereby the arms 26U, 26L return to their original OPEN position, due to the resilient nature of the stainless steel material. Not only will this clip 26 last for ever, but it is very easy and cheap to manufacture.

Air Hose/Clip Connector

FIGS. 24A-24D show a perspective view, a side view, a top view and a bottom view of an air hose/clip connector according to the present invention.

Referring to the FIGS. 24A-24D, numeral 260 generally designates the air hose/clip connector 260 (hereinafter referred to as hose connector 260).

The hose connector 260 comprises a round cylindrical portion 260c having a through hole 260h formed through the center thereof, a cone shaped protrusion 260v formed on the outer central surface of the cylindrical portion 260c and a disc like base portion 260b formed on the cylindrical portion along one end thereof. The larger diameter portion of the cone shaped protrusion faces the disc like protrusion. The outer diameter of the cylindrical portion 260c is the same as the diameter of the hole 26h2 in the clip 26.

To assemble the hose connector 260 in the clip 26, the extending portion of the cylindrical portion 260c is pulled through the hole 26h2 until the cone shaped protrusion pups out of the top of the hole 26h2 (as shown in FIGS. 23F and 23G) to permanently lock the hose connector 260 in the clip 26. The air hose 14 can be mounted on the extending end of the cylindrical portion 260c.

The hose connector is made of resilient plastic, rubber, latex, silicone or any other suitable material.

Stretchable Single Decker Air Bag

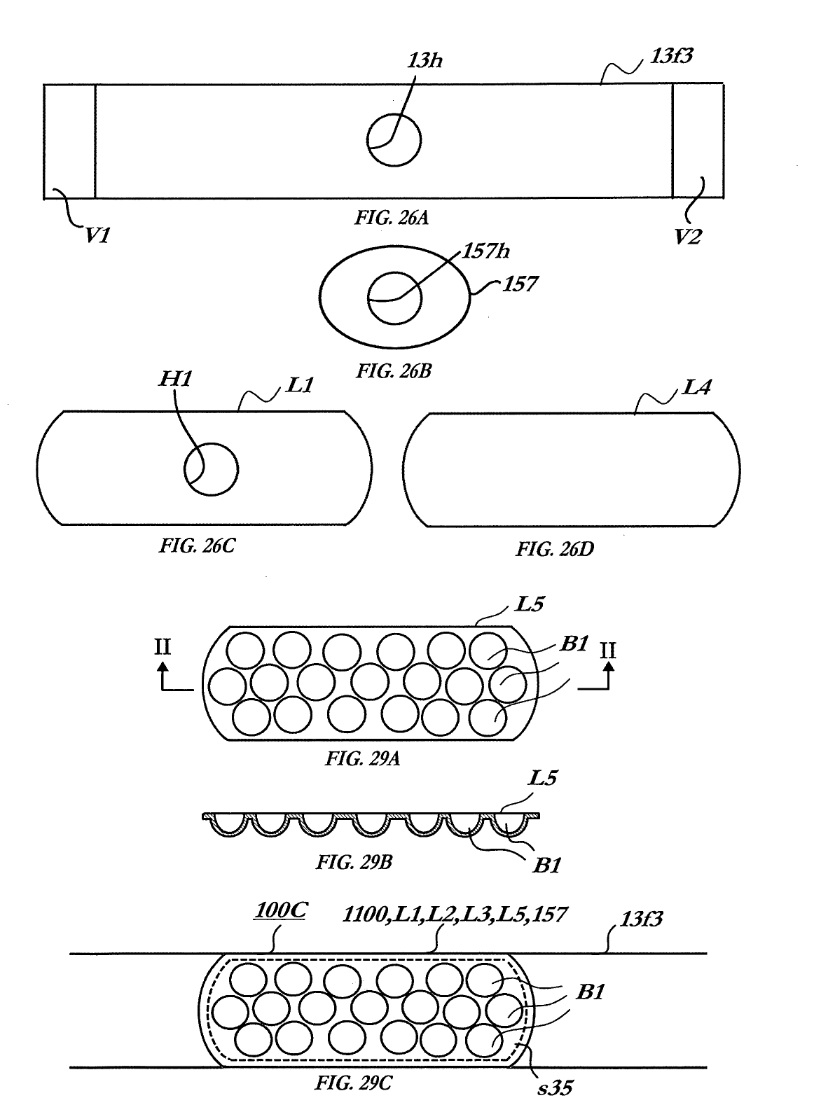

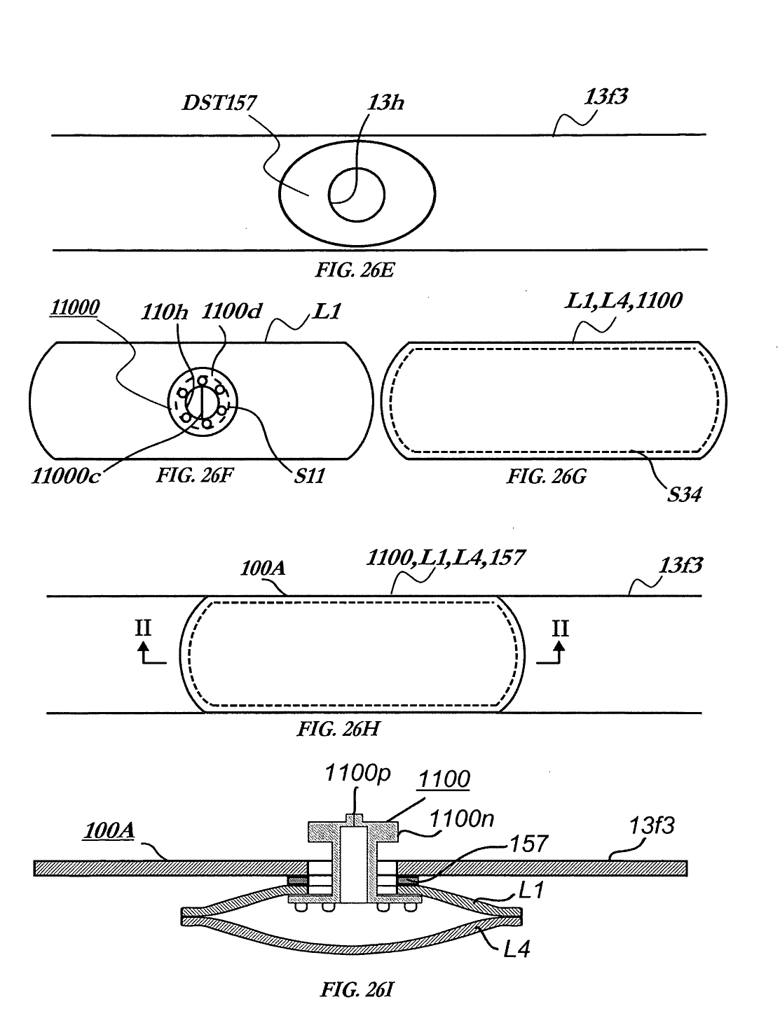

FIGS. 26A-26D show front view of the parts needed to make the single decker air bag 100A. FIGS. 26E-26H show the steps required to manufacture the single deck air bag 100A according to the present invention. FIG. 26I shows a side cross sectional view of the single decker air bag 100A at line II-II in FIG. 26H.

Referring to FIGS. 26A-26I, numeral 13f3 designates a strap made of a bendable but not stretchable plastic film such as polyethylene and has a male and a female. Velcro strips V1, V2 bonded to the respective ends of the strap 13f3, on opposite sides of the strap 13f3, so that they can be connected to each other when the band 22 is wound around a persons wrist. Numeral 13h designates a round hole punched out or cut out of the center of the band 13f1 for accommodating the head 1100n of the nipple 1100 therethrough.

FIG. 26B shows a DST 157 having a hole punched out through the center thereof for accommodating the head 1100n of the nipple 1100 therethrough. The DST157 is used to bond the double deck air bag to the strap 13f3.

The double deck air bag comprises two layers of stretchable and bendable plastic film sheets, L1 and L4. The film sheets L1, L4 are rectangular or oval in shape and are about 4-8 cm. long and 3-6 cm wide. The sheets L1, L4 are made of polypropylene, silicone, latex or any other material which is stretchable. Preferably, the sheets L1, L4 are about about 20 percent stretchable.

To manufacture the air bag 1000, first 2 identical oval shaped pieces of thin polypropylene sheets L1, L4 are punched out or cut out of a large polypropelene sheet. The sheet L1 also has a hole H1 punched or cut out of the center thereof. The size of the hole H1 is the same as the size of the head 1100n of the nipple 1100.

Next, as shown in FIG. 26F, the head portion 1100n of the nipple 1100 is passed through the hole H1 in the layer L1 and then the base portion 1100d of nipple 1100 is bonded to the layer L1 using conventional heat sealing techniques as shown by dash lines S11. Alternatively, any conventional boding techniques, such as heat boding, plastic adhesives, double sided tape, etc. may be used to bond the base 1100n to the sheet L1.

Next, as shown in FIG. 26G, the two layers L1 and L4 are bonded to each other along the peripheries thereof using conventional heat sealing techniques, as shown by dash line S34. Next, as shown in FIG. 26E, the DST157 is bonded to the strap 13f3 with the holes 157h in the DST157 and the hole 13h in the strap 13f3 aligned with each other. Next, the head portion 1100n of the nipple 1100 is passed through the holes 13h and 157h and the sheet L1 is bonded the other side of the DST 157.

Stretchable Double Decker Air Bag

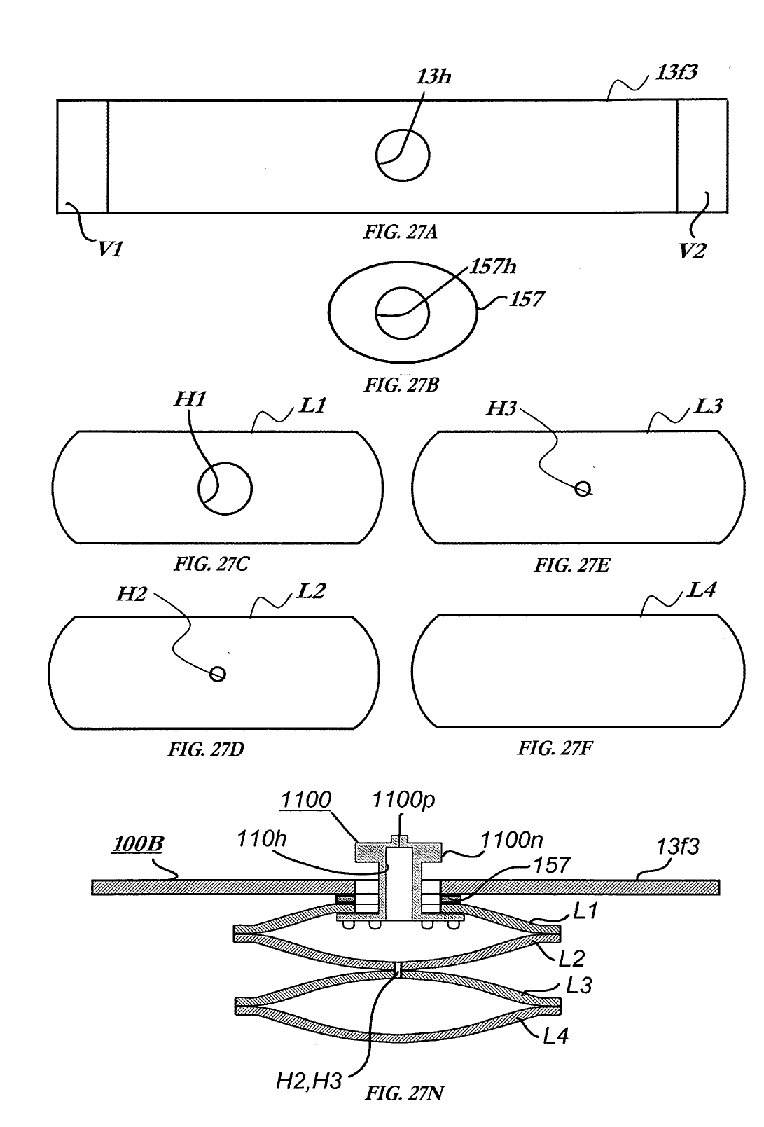

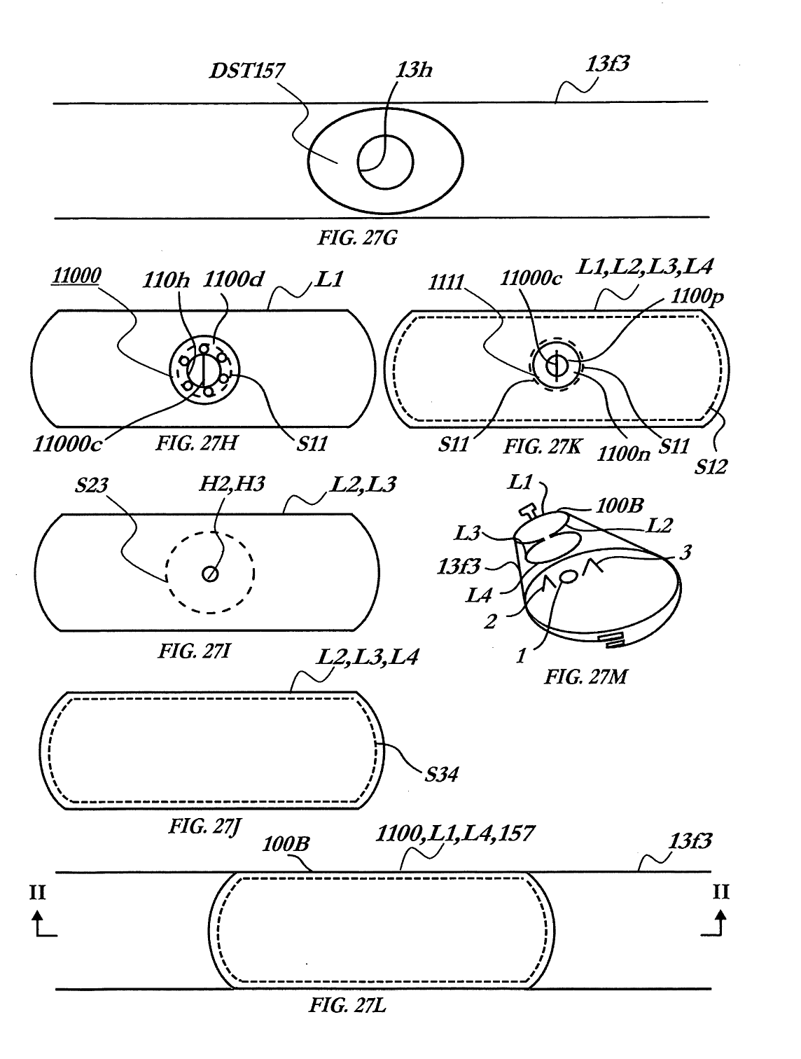

FIG. 27M shows a schematic view of a double-deck air bag 100B for a blood pressure measuring device according to another embodiment of the present invention. The double decker air bag 100B is similar to the single deck air bag 100B and only the differences therebetween will be described herebelow. The main advantage of having the double decker air bag 100B over the single deck air bag 100A is that the double decker air bag 100B can take up more slack in the strap 13f3. Accordingly, even if the band 100B is mounted very loosely around a persons arm, the double deck air bag 100B can take up the slack.

FIGS. 27A-27F show front views of all the parts needed to make the double deck air bag 100B. FIGS. 27G-27L show the steps required to manufacture the double deck air bag 100B according to the present invention. FIG. 27N shows a side cross sectional view of the double deck air bag 100B at line II-II of FIG. 27L.

Referring to FIGS. 27A-27L, numeral 13f3 designates a strap made of a bendable but not stretchable plastic film such as polyethylene and has a male and a female Velcro strips V1, V2 bonded to the respective ends of the strap 13f3, on opposite sides of the strap 13f3, so that they can be connected to each other when the strap 13f3 is wound around a persons wrist. Numeral 13h designates a round hole punched out or cut out of the center of the strap 13f3 for accommodating the head 1100n of the nipple 1100 therethrough.

FIG. 27B shows a DST 157 having a hole punched out through the center thereof for accommodating the head 1100n of the nipple 1100 therethrough. The DST 157 is used to bond the double deck air bag to the strap 13f3.

The double deck air bag comprises four layers of stretchable and bendable plastic film sheets, L1, L2, L3 and L4. The film sheets L1-L4 are rectangular or oval in shape and are about 4-8 cm. long and 3-6 cm wide. The sheets L1-L4 are made of polypropylene, silicone, latex or any other material which is stretchable. Preferably, about 20 percent stretchable.

To manufacture the air bag 100B, first 4 identical oval shaped pieces of thin polypropylene sheets L1-L4 are punched out or cut out of a large polypropelene sheet. Next, as shown in FIGS. 27D, 27E two of the sheets L2 and L3 have a small hole H2, H3 punched or cut out along the center thereof. Next, as shown by the dash line S23 in FIG. 27I, the two layers L2 and L3 are bonded to each other around the holes H2, H3 using conventional heat sealing techniques. Next, as shown in FIG. 27C, a hole H1 is punched or cut into the center of the sheet L1, the diameter of the hole H1 being the same as or slightly larger than the outer diameter of the head portion 1100n of the nipple 1100. Next, as shown in FIG. 27H, the head portion 1100n of the nipple 1100 is passed through the hole H1 in the layer L1 and then the base portion 1100d of nipple 1100 is bonded to the layer L1 using conventional heat sealing techniques as shown by dash lines S11. Alternatively, any conventional boding techniques, such as heat boding, plastic adhesives, double sided tape, etc. may be used to bond the base 1100n to the sheet L1. Next, as shown in FIG. 27J, the sheets L3, L4 are heat bonded to each other along the peripheries thereof (as shown by the dash line S34). At this time, the outer portion of the layer L2 are folded inward or just kept out of the way of the layers L3 and L4. Next, as shown in FIG. 27K, the layers L1 and L2 are heat welded to each other along the peripheries thereof (as shown by dash lines S12).

Next, as shown in FIG. 27G, the DST157 is bonded to the strap 13f3 with the holes 157h in the DST157 and the hole 13h in the strap 13f3 aligned with each other. Next, the head portion 1100n of the nipple 1100 is passed through the holes 13h and 157h and the sheet L1 is bonded the other side of the DST 157.

Accordingly, with this double sided air bag 100B, when the air bag 100B is blown up with air, the bag 100B expands twice as far towards the artery 1 (shown in the schematic drawing FIG. 27M) to take up more slack in the band than otherwise possible.

Stretchable Double Decker Air Bag with Bubble Surface

FIG. 29A show a front view of a rectangular shaped film sheet of plastic material L5 having a plurality of semi spherical protrusions B1 (hereinafter referred to as bubbles B1) formed along the surface thereof. FIG. 29B shows a cross sectional view at line II-II in FIG. 29A of the film L5. FIG. 29C shows a front view of a double decker air bag 100C having the bubble sheet L5 as the outermost sheet (i.e. the sheet L4 has been replaced by the bubble sheet L5) according to another embodiment of the present invention. The double decker air bag 100C is the same as the double decker air bag 100B, with the only difference being that instead of using the sheet L4 described above, the bubble sheet L5 is used by heat sealing the bubble sheet L5 to sheet L3 along the peripheries thereof, as shown by dash line S35.

Since the air bubbles are staggered along the front surface of the sheet L5, there is a very strong chance that at least one of the bubbles will push directly on the artery 1 when the air bag 100C is inflated with air. The bubbles should be about 5-10 mm in diameter. With this air bag 100C, the sheets L1, L2, L3 and L5 can be made using bendable material and either stretchable or non-stretchable material. Since the bubbles B1 are less than 10 mm in diameter, they can easily fit between the radius bone 2 and the digital tendon 3 and push against the artery 1.

The bubble sheet L5 is commonly used for wrapping electrical goods for the physical protection of the electrical goods.

Stretchable Air Bag 1000A

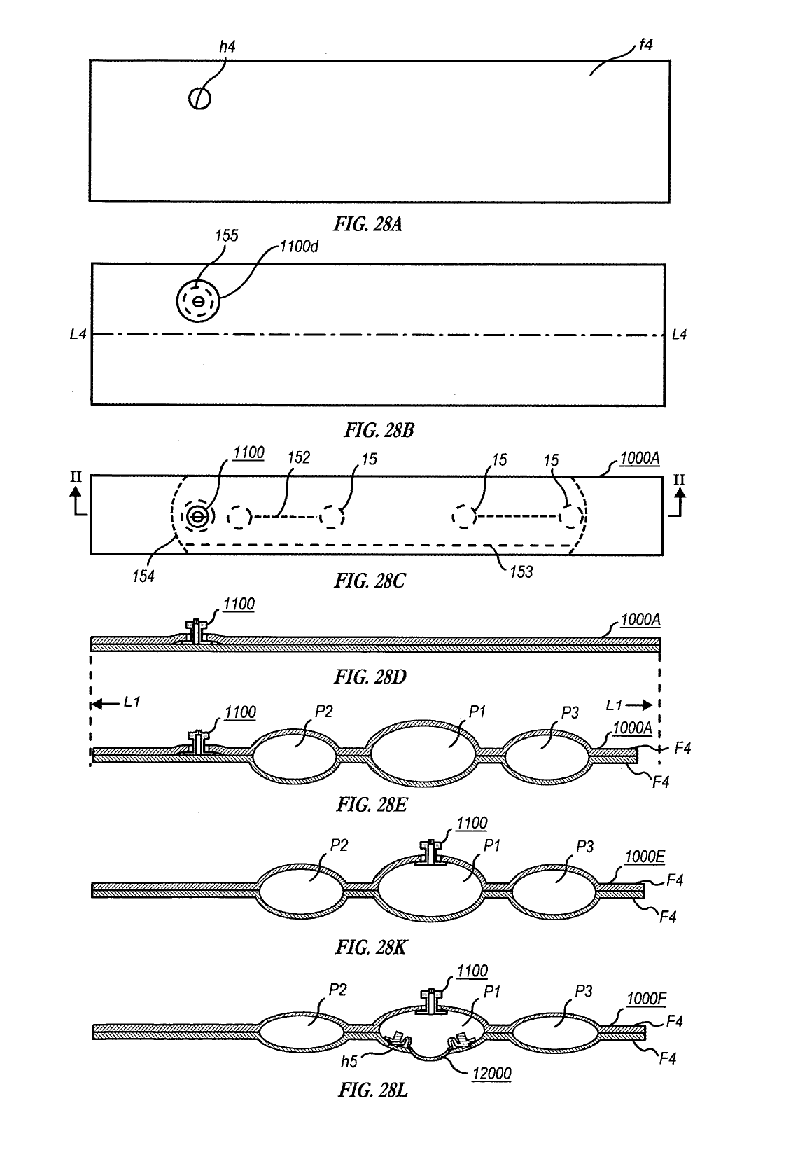

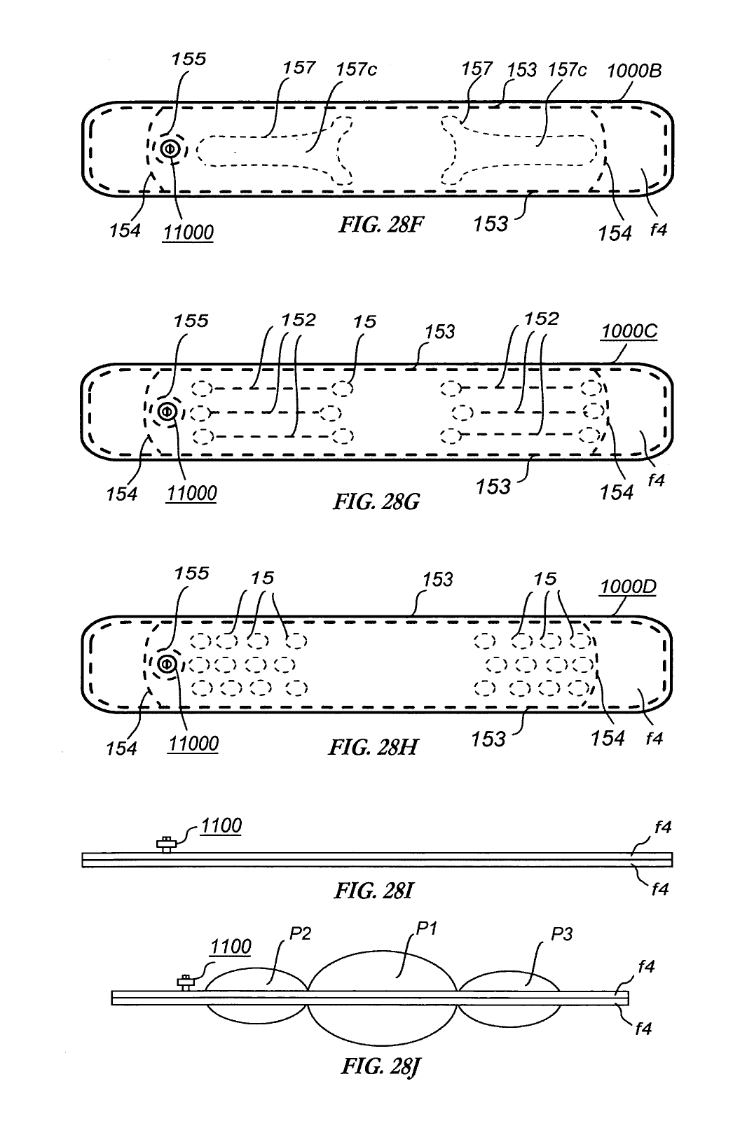

FIGS. 28A-28C show the parts used in the manufacture of a stretchable air bag 1000A and the steps to manufacture the same according to another embodiment of the present invention. Referring to the Figs., numeral f4 designates a film of stretchable and bendable plastic material such as polypropylene, rubber, latex, silicone, etc. The film f4 is cut in the shape of a rectangle and is about 30 cm. long (i.e. long enough to fit around a person's wrist) and 6-10 cm. wide. Numeral h4 designates a round hole punched out of the film f4 along one end thereof. The diameter of the hole h4 is the same as the diameter of the head 1100n of the nipple 1100. The hole h4 is also formed towards one of the sides of the film h4, so that when the film is folded in half, the hole h4 is at the center of the folded film with respect to the width thereof. Next, as shown in FIG. 28B, the head of the nipple 1100 is placed through the hole h4 and then the base portion 1100d of the nipple 1100 is heat sealed, as shown by dash lines 155, to the film f4. Next, the film f4 is folded in half along the length thereof at the dot and dash line L4-L4 with the head 1100n of the nipple 1100 facing outwards. Next, the thus folded film is heat sealed along the surface thereof as indicated by dash lines 152, 153 and 154. Numerals 15 designate round heat welds formed at both ends of each of the heat welds 152 and are provided for distributing the air pressure stress forces produced when the air bag 1000A is inflated.

FIG. 28D and 28E show cross sectional views of the air bag 1000A at line II-II in FIG. 28C with no air and with air therein, respectively. It can be seen that the air bag 1000A when inflated forms three pockets of air P1, P2 and P3 along the length thereof. The air pocket P3 is much larger than the air pockets P2, P3, due to the way the heat seals 152-154 are formed. This is desirable because the pocket P1 is the one that is placed over the radial artery. Furthermore, as the air volume inside the air bag 1000A is increased, the length L of the air bag 1000A decreases, resulting in the air bag 1000A automatically tightening around the persons wrist. This is desirable in order to take up any slack which may exist between the persons wrist and the air bag 1000A. The extending ends of the air bag 1000A may have male and female Velcro parts mounted thereon, or any other conventional means of joining the ends to each other.

FIGS. 28F, 28G and 28H show three more embodiments of air bags 1000B, 1000C and 1000D according to the present invention. FIGS. 28I and 28J show side views of the air bags 1000B-1000D with no air and with air inside the bags, respectively.

The air bags 1000B-1000C are similar to the air bag 1000A and only the differences therebetween will be described herebelow.

Referring to FIGS. 28F-28H, instead of using one sheet f4 and folding it, as in the case of the air bag 1000A, two sheets f4 are placed on top of each other and heat welded along the peripheries thereof as shown by dash lines 153. With this embodiment, the outer sheet f4 in which the nipple 155 is mounted can be made thicker than the inner sheet f4 which is the sheet coming into contact with the persons arm, so that a more sensitive systolic and diastolic measurement can be obtained. Furthermore, with the outer layer f4 being thicker than the inner layer f4, the air bags 1000B-1000C would not inflate as much outwards and accordingly, less air would be required to obtain the systolic and diastolic blood pressures.

The air bag 1000B shown in FIG. 28C has two substantially oval shaped heat welds 157 formed on either side thereof with respect to the center thereof. The heat welds 157 not only produce the two smaller air pockets P2, P3, but also the center portions thereof 157c can be cut out to provide air circulation to avoid patients from sweating.

The air bag 1000C shown in FIG. 28G has a plurality of heat weld 152 formed on either side of the air bag 1000C which produce a plurality of small air pockets P2 on either side of the air bag 1000D.

The air bag 1000D has a plurality of rows of round heat welds 15 formed on either side of the air bag which produce a plurality of smaller air pockets P2 on either side of the large air pocket P1.

Each of the air bags 1000A-1000D described above provide advantages such as air ventilation, comfort, ease of use and manufacture, as well as being very simple cheap and easy to use, and accordingly, can be used as disposable air bags in hospitals. A small pocket for entering the name of a patient can easily be incorporated into the air bags 1000A-1000B as described above with respect to the air bag 10B (FIG. 2J)

FIGS. 28I-28J show side views of the air bags 28F-28H with no air and in the inflated mode.

FIG. 28K shows a side cross sectional view of an air bag 1000E according to another embodiment of the present invention. The air bag 1000E is the same as any of the air bags 1000A-1000D with the exception of having the nipple 11000 mounted in the middle of the air bag where the big air pocket P1 is formed.

FIG. 28L shows a side cross sectional view of an air bag 1000F according to another embodiment of the present invention. The air bag 1000F is the same as the air bags 1000E with the exception of having the diaphragm 12000 mounted in a oval hole in the inner film f4 formed in the middle of the air bag where the big air pocket P1 is formed.

RFID Clip

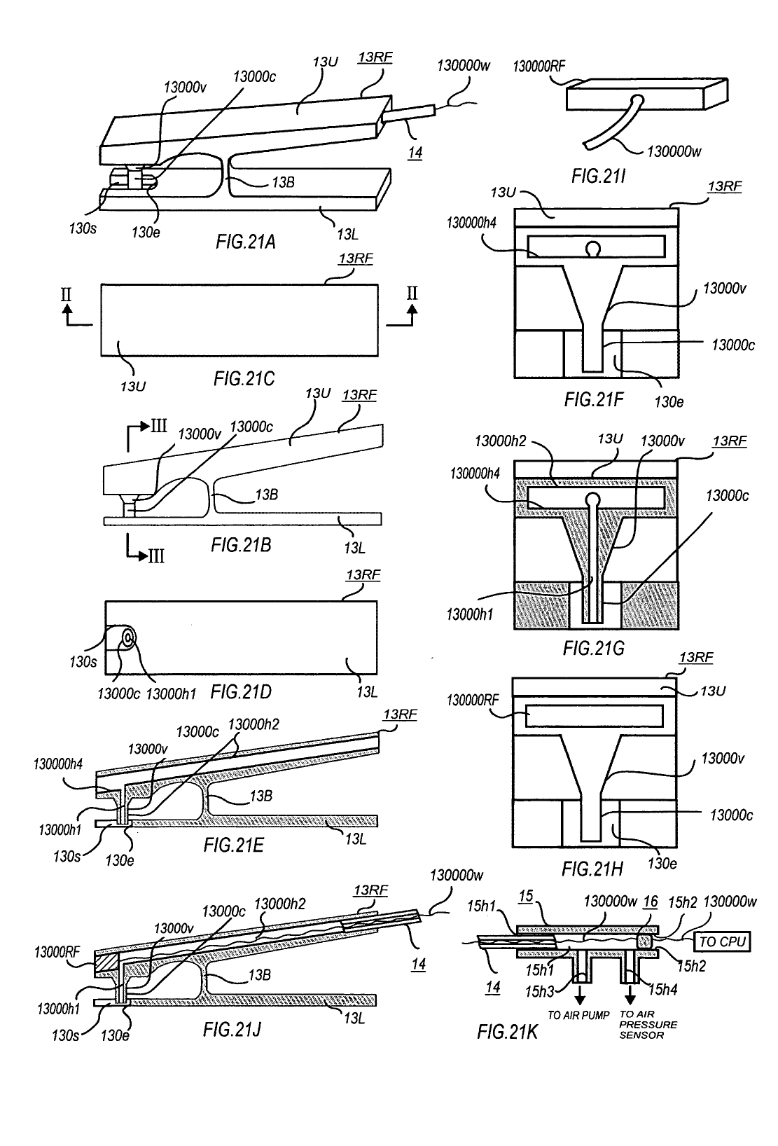

FIG. 21A shows a perspective view of a clip 13RF (hereinafter referred to as RF clip 13RF) having a radio frequency reader (hereinafter referred to as RFR 130000RF) mounted therein for sending patient identification information to the electronic blood pressure measuring device to which the clip 13RF is connected to.

FIG. 21B-21E show a side view, a top view, a bottom view and a side cross sectional view at line II-II in FIG. 21C of the RF clip 13RF.

FIG. 21F, shows a front view of the RF clip 130000.

FIG. 21G shows a cross sectional view at line III-III of FIG. 21B of the RF clip 13RF.

FIG. 21H shows a front view of the RF clip 13RF with the RFR 130000 RF mounted therein.

FIG. 21I shows a side cross sectional view at line III-III in FIG. 21C of the RF clip 130000 having the RFR 130000RF mounted therein.

FIG. 21I shows a perspective view of a RFR 13000RF according to the present invention. FIG. 21J shows a side cross sectional view at line II-II in FIG. 21C of the RF clip 13RF with the RFR 130000RF mounted therein.

FIG. 21K shows a side cross sectional view of a plastic coupling device 15 for connecting the air hose 14 and the electrical wires 13000w to the electronic blood pressure measuring device (not shown) according to the present invention.

The RF clip 13RF is similar to the clip 13000 and only the differences therebetween will be described herebelow.

Referring to the FIGS. 21A-21K, numeral 130000h4 designates a rectangular shaped cavity formed in the upper arm 13U. The cavity 130000h4 extends from the front central portion of the upper arm 13U to the point where the horizontal hole 13000h2 and vertical hole 13000h1 join. The size and shape of the cavity 130000h4 is the same as the outer size and shape of the RFR130000RF. Accordingly, the RFR130000RF frictionally fits into the cavity 130000h4, as shown in FIGS. 21H and 21J. When mounting the RFR 130000 RF inside the cavity 130000h4, first the electrical wires 130000w coming out of the back of the RFR 130000RF are inserted into the cavity 130000h4 and then pulled through the horizontal hole 13000h2, through the inside of the air hose 14 and then into a plastic coupling device 15 (hereinafter referred to as PCD 15). The PCD 15 is mounted inside an electronic blood pressure measuring device (not shown). The outer surface of the RFR 13000RF is then pushed into the square cavity 130000h4 to form a hermetic seal therebetween. The outer surface of the RFR 130000RF can have an adhesive applied thereto to glue the RFR 130000RF to the inner surface of the cavity 130000h4, thereby creating a hermetic seal therebetween.

The PCD 15 comprises a plastic body having four round holes 15h1-15h4 formed therein, the four holes being connected to each other inside the PCD 15. The other end of the hose 14, having the electrical wire 130000w inserted therein is frictionally inserted into a first round hole 15h1 in the PCD 15. The electrical wires 13000w are passed through inside of the PCD 15 and out of the PCD 15 through the hole 15h2. Then a silicone sealant 16 is inserted into the hole 15h2 to hermetically seal the wires 13000w inside the hole 15h2. An air pump (not shown) and an air pressure sensor (not shown) of an electronic pressure measuring device (not shown) are hermetically connected to the other hole 15h3 and 15h4, respectively.

It should be noted that instead of the RFR 13000RF any other electronic identifying device may be used such as a bar code scanner, etc.

Accordingly, with the RF clip 13RF, a bar code or a RFID device can be inserted together with the paper 10ID having the name of the patient into the pocket 100p in the air bag 10B shown in FIG. 2J (i.e. on the paper 10ID having CHARLIE CHAPLIN written on it with RFID device mounted on the paper 10ID just adjacent to the nipple 11) or, as shown in FIG. 11L, the pocket p2 in the air bag 100000A (i.e. on the paper 11ID having Marilyn Monroe written on it with the RFID device mounted on the paper 10ID just adjacent to the nipple 110000). The bar code or the RFID device can be placed in the pocket 100p and p2 in the air bags 10B and 100000A at a position directly below the point where the RFR 130000RF will be when the RF clip 13RF is mounted on a unidirectional nipple 110000 which will be described herebelow.

Unidirectional Nipple

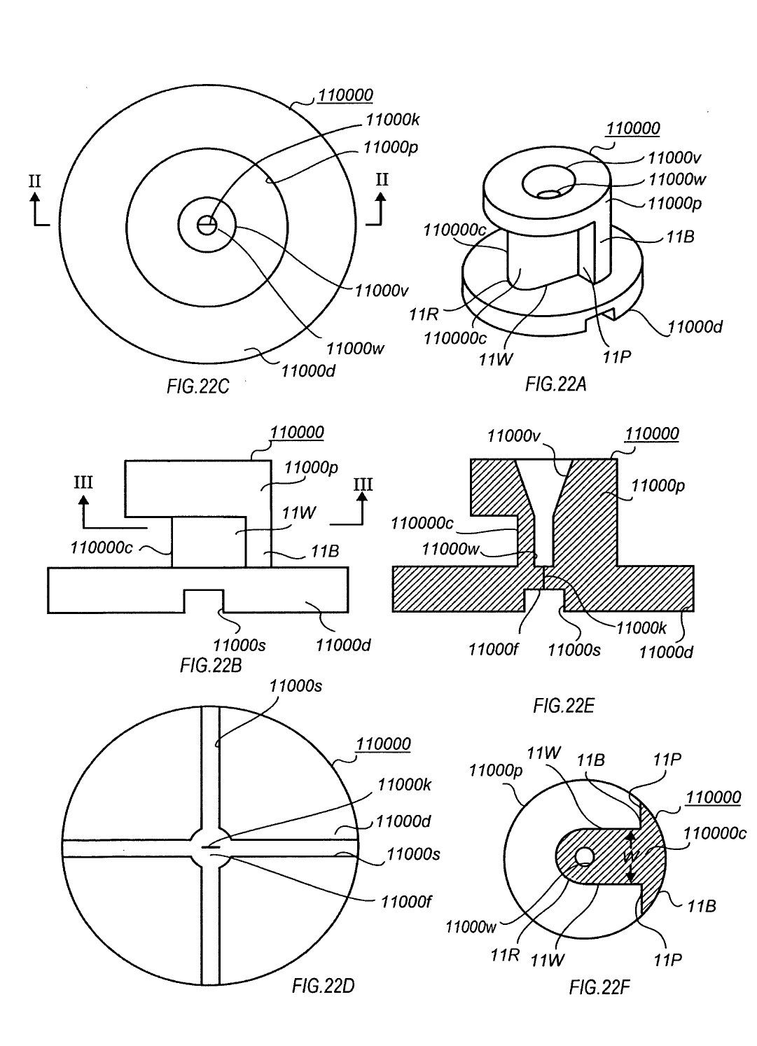

FIGS. 22A-22F show a perspective view, a side view, a top view, a bottom view, a side cross sectional view at line II-II of FIG. 22C and a cross and a cross sectional view at line III-III of FIG. 22B of a nipple 110000 according to another embodiment of a unidirectional nipple according to the present invention.

The nipple 110000 is similar to the nipple 11000 and only the difference therebetween will be described herebelow. The nipple 110000 is a unidirectional nipple which allows the clip 13RF to be mounted thereon in only one direction.

Referring to the FIGS. 22A-22F, and particularly to FIG. 22F, numeral 110000c designates a shaft portion having the head portion 11000p and the base portion 11000d integrally formed therewith at opposite ends thereof, respectively.

The shaft 110000c comprises a semi round back portion 11B having the same diameter as the head portion 11000p, a rectangular shaped middle portion 11W the with W of which is less than the diameter of the head portion 11000p and a semi-round front portion 11R the diameter of which is the same as the width W of the middle portion, the rectangular middle portion 11W and semi round front and back portions 11R and 11B being integrally formed with each other.

Numeral 11000w designates a round hole formed at the center of the shaft portion 110000c. Numeral 11P designates two perpendicularly extending walls on either side of the of the rectangular section 11W. The perpendicular walls 11P join the walls of the rectangular section 11W and the walls of the semi-round back portion 11B. The width W of the rectangular section 11W is the same as the width W of the slot 130s formed in the bottom arm 13L of the RF clip 13RF. Accordingly, with this nipple 110000 (hereinafter referred to as unidirectional clip 110000 or UD clip 110000), the RF clip 13RF can only be mounted on the UD nipple 110000 in one direction, namely, from the side where the semi-round front portion 11B is formed. The RF clip 13RF can be mounted on the UD nipple 110000 up to the point where the front end of the lower arm 13L of the RF clip 13RF comes into contact with the perpendicular walls 11P of the shaft 110000c, at which point the cone shaped portion 13000v and cylindrical portion 13000c of the RF clip 13RF are respectively aligned with the holes 11000v and 11000w in the UD nipple 110000. At this position, the front portion of the round portion 11R also buts up against the back of the slot 130s in the RF clip 13RF. Furthermore, the RF clip 13RF cannot be mounted from the back side of the UD nipple 110000 where the semi-round shaft back portion 11B is formed, since the slot 130s in the RF clip 13RF is narrower than the widest part of the back portion 11B (i.e. the extending ends of the perpendicular walls 11P).

Accordingly, the RF clip 13RF can be mounted on the UD nipple 110000 in one direction only.

Disposable Air Pressure Belt with Diaphragm and Electronic Pressure Measuring Device Mounted Therein.

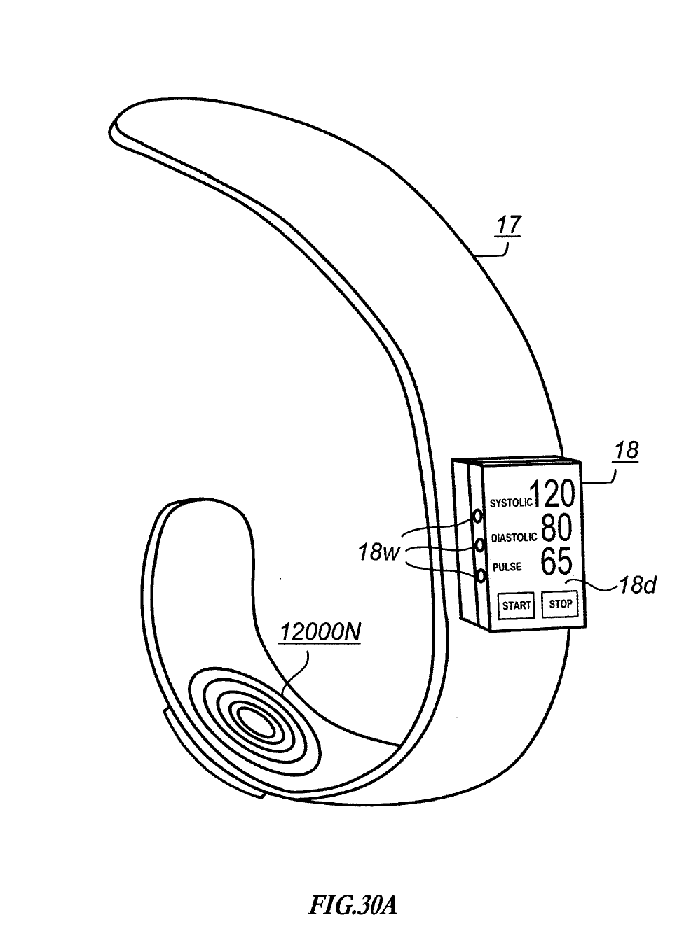

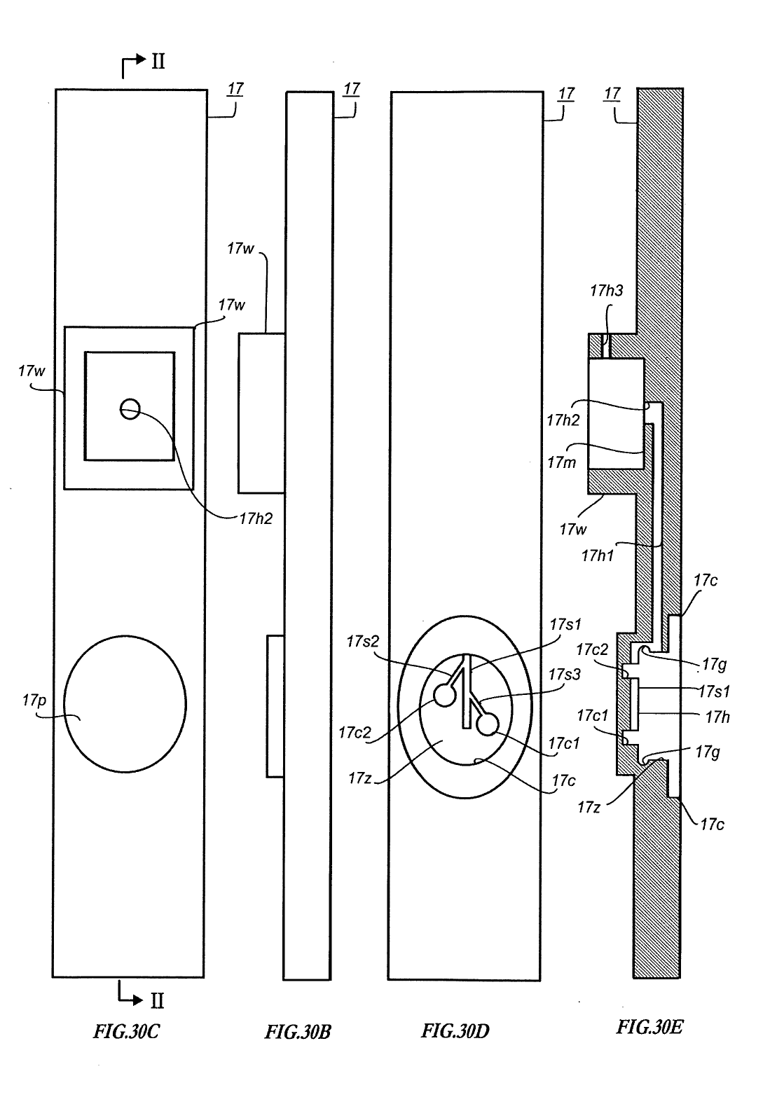

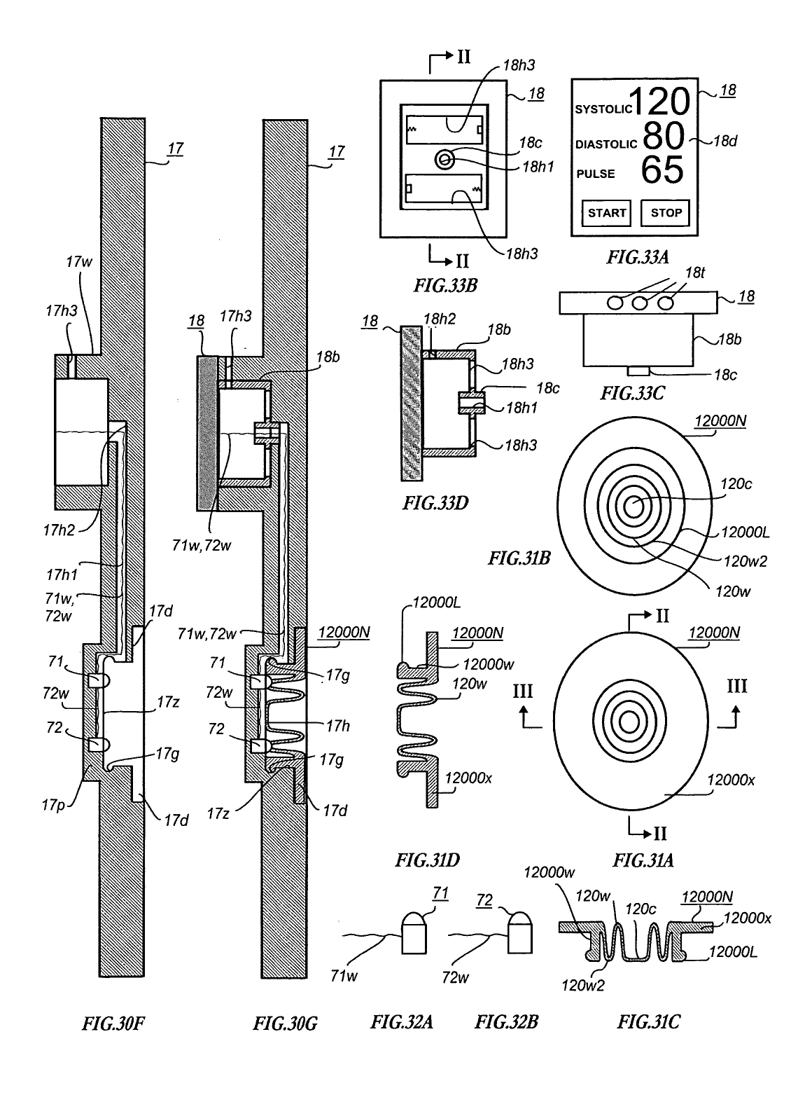

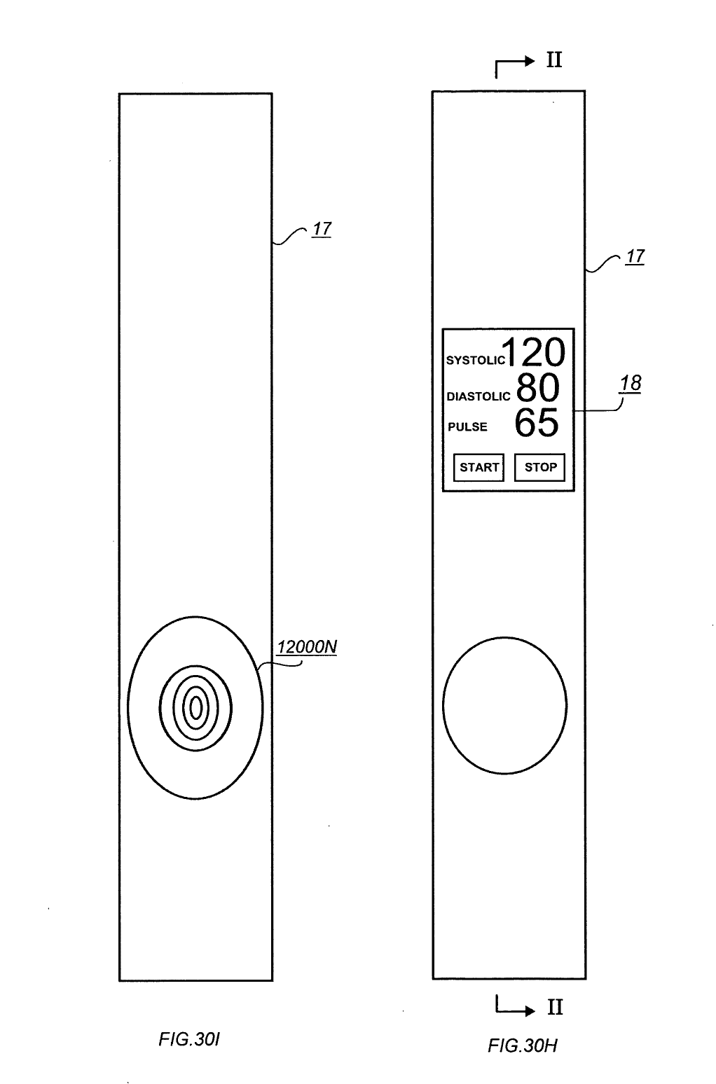

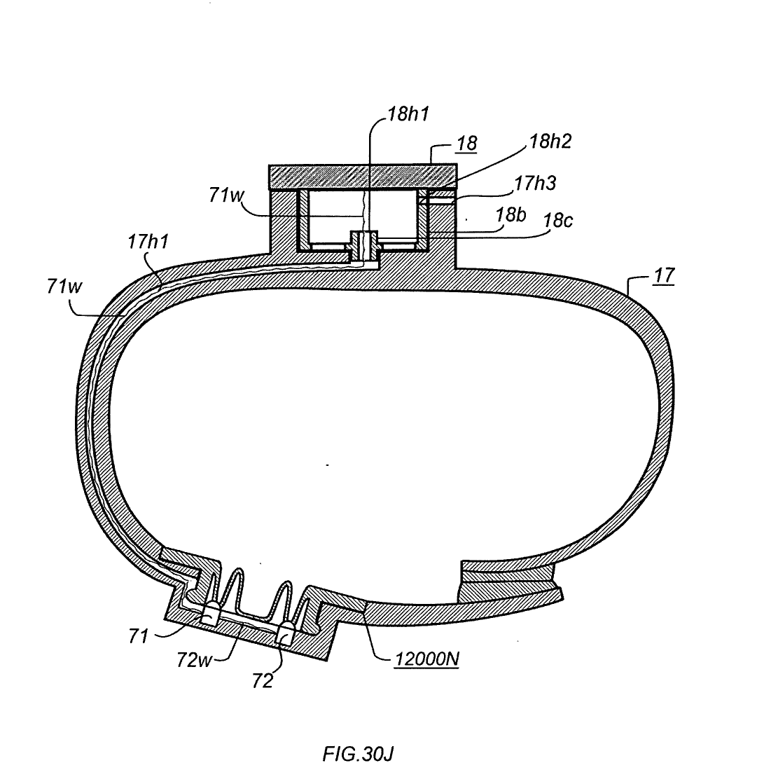

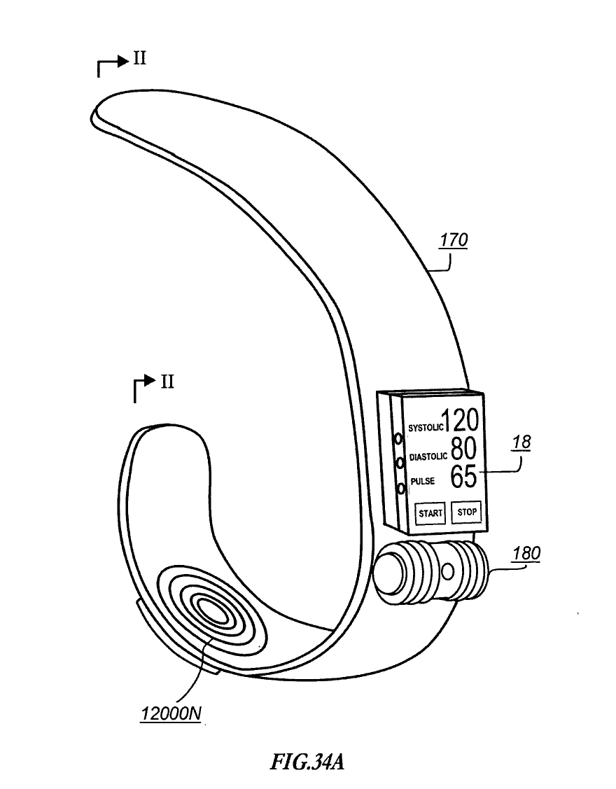

FIG. 30A shows a perspective view of a disposable air pressure belt 17 having a diaphragm 12000N and electronic pressure measuring device 18 mounted therein.

FIG. 30B-30E show a side view, a top view, a bottom view and a side cross sectional view at line II-II in FIG. 30C of a bendable but not stretchable band 17 for a blood pressure measuring device according to the present invention.

FIG. 30F shows a cross sectional view of the belt at line II-II in FIG. 30F having a light emitting diode LED 71 and a photo sensor 72 mounted therein.

FIG. 30G shows a cross sectional view of the belt at line II-II of FIG. 30C further having a diaphragm 12000N and an electronic blood pressure measuring device 18 mounted therein.

FIGS. 30H, 30I show a front view afiffnd a back view of the disposable air pressure belt 17 having a diaphragm 12000N and electronic pressure measuring device 18 mounted therein.

FIG. 30J shows a side cross sectional view of the belt at line II-II of FIG. 30C having a diaphragm 12000N and an electronic blood pressure measuring device 18 mounted therein, the belt 17 being bent in a circle.

FIG. 31A-31D show a front view, a back view and cross sectional views at lines II-II and III-III of a diaphragm 12000N according to another embodiment of a diaphragm according to the present invention.

FIG. 32A, 32B show side views of a conventional light emitting diode LED 71 and a photo sensor 72.

FIG. 33A-33D show a front view, a side view, an end view and a cross sectional view at line II-II in FIG. 33B of a plastic box for containing an electronic blood pressure measuring device.

Blood Pressure Belt

Referring to FIGS. 30A-30J, numeral 17 generally designates an arm band made of rubber, nylon, or any other flexible material. The band 17 is thin and wide and long enough to go around a persons arm. Accordingly, the band 17, although being easily bendable around a persons arm, is substantially not stretchable under normal forces. Numeral 17w designates a square wall integrally formed with the band 17 along the outer surface of the band 17. The wall 17w and the top surface of the band 17 inside the wall 17 together produce a first cavity which serves to accommodate an electronic blood measuring device 18 therein. Numeral 17c designates an oval shaped second cavity formed on the inner surface of the band 17. The oval shaped second cavity 17c serves to support a diaphragm 12000N therein.

It should be noted that the wall 17w can be formed in an oval shape in the case where the electronic blood pressure measuring device 18 is oval in shape. Namely, the wall 17w can be formed to have the size and shape of any electronic blood pressure measuring device.

Numeral 17h2 designate a vertical hole extending from the outer surface of the band 17 to a point between the inner and outer surfaces of the band 17. The hole 17h2 is located inside the square wall 17w at a central point thereof. Numeral 17h1 designates a round hole which extends from the inner side of the hole 17h2 horizontally along the inside the belt 17 along the length direction of the belt 17 to the inner side wall 17z of the oval cavity 17c. Accordingly, air from a central point inside the wall 17w can easily pass to the inside of the oval cavity 17c.

Numeral 17s1, 17s2 and 17s3 designate three slots formed in the inner wall 17b of the cavity 17c. Numeral 17c2 and 17c3 designate two round cavities for mounting the respective back end of the LED 71 and the photo sensor 72 therein. The electrical wires 71w and 72w from the LED 71 and photo sensor 72 are inserted in the slots 17s2 and 17s3 and 17s1, and then through the holes 17h1, 17h2 into the center of the bottom surface 17m inside the square wall 17w. Then the wires 71w and 72w are fed through the hole 18h1 into the box 18b, where the electronic pressure measuring device (not shown) is housed. The LED 71 and photo sensor 72 are well know in the art and are commonly used for measuring pulse rate, etc.

Referring to FIGS. 31A-31D, the diaphragm 12000N is similar to the diaphragm 12000 and only the differences therebetween will be described herebelow. Numeral 12000L designates a oval shaped lip integrally formed along the outer surface of the diaphragm wall 12000w at the extending end thereof. The diaphragm 12000N and the diaphragm 12000 are the same in all other aspects.

The size and shape of the outer side of the wall 12000w of the diaphragm 12000N is the same as the inner size and shape of the side walls of the 17c, so that the wall 1200w snugly fits against the side walls of the cavity 17c, thereby forming an air tight seal therebetween.

Numeral 17g designates a oval shaped groove formed in the side wall 17z along the inner side of the wall 17z. The size and shape of the groove 17g in the wall 17z is the same as the size and shape of the lip 12000L along the outer surface of the wall 12000w of the diaphragm 12000N.

To mount the diaphragm 12000N in the cavity 17c in the band 17, the diaphragm 12000N is pushed into the cavity 17c until the lip 12000L at the bottom of the diaphragm 12000N snaps into the groove 17g in the cavity 17c of the band 17, thereby hermetically locking and sealing the diaphragm 12000N in the cavity 17c.

To ensure that the diaphragm 12000N is secured in the cavity 17c, glue may be applied to the side surfaces 17z of the cavity 17c as well as to the depression 17d of the band 17. Next, the diaphragm 12000N is inserted into the cavity with the diaphragm wall 1200w facing into the cavity 17c. However, it may be desirable to change the diaphragm in case of tearing or damage, and accordingly, glue may not be desirable. In the case of damage to the diaphragm 12000N, it can be easily pulled out of the cavity 17c and replaced with a new diaphragm.

The shape and size of the outer wall 12000w of the diaphragm 12000N are the same as the inner side walls of the cavity 17c, so that the walls 12000w of the diaphragm 12000N frictionally fit therein.

The diaphragm 12000N should be made of transparent stretchable material such as rubber, silicone latex, etc., so that light from the LED 71 can pass therethrough and, after being reflected in the arm of a person, be detected by the photo sensor 72.

Referring to FIGS. 33A-33D, the electronic blood measuring device 19 comprises a outer square plastic case 18, a conventional display 19d which is mounted on the case 18 using any conventional method (i.e. glue, screws, etc) and a conventional electronic control system (not shown) inside the case 18. The case 18 is substantially square and has outer dimensions which are identical to the inner dimensions of the inner side of the wall 17w of the band 17, so that the box 18 snugly fits inside the walls 17w. The bottom surface of the case 18 has a round short cylindrical portion 18c formed therewith, the cylindrical portion 18c partially extends into the case 18 and partly out of the case 18.

The cylindrical portion 18c has an outer diameter which is slightly bigger than the inner diameter of the cylindrical hole 17h2 formed in the belt 17, so that when the case 18 is mounted inside the wall 17w in the belt 17, the outer end of the cylindrical portion 18c snugly fits inside the hole 17h2 in the band 17, and accordingly, forms a hermetic seal therebetween.

The inner end of the cylindrical portion 18c inside the case 18 is hermetically connected to a conventional air pump (not shown), a pressure sensor (not shown) and the wires 71w, 72w are connected to the CPU of the electronic blood pressure measuring device (not shown), by using the connector 15 (shown in FIG. 21K).

Accordingly, when the START button on the surface of the display 18d of the electronic blood pressure measuring device 18 is pressed, air from the air pump (not shown) passes through the hole 170h in the cylindrical portion 18c1 through the holes 17h2, 17h1,through the slot 17s1 and into the cavity 17c, and accordingly, causes the diaphragm 12000N to fill with air to cause the waves 120w and 120w2 to unfurl and force the central portion 120c of the diaphragm 12000N to press outwards towards the radial artery.

Numeral 18h2 designates a small hole formed in one of the sides of the square box 18 and numeral 17h3 designates a hole formed in the side of the wall 17w of the band 17. When the box 18, having the electronic blood pressure measuring device (not shown) mounted therein, is inserted into the space inside the wall 17w of the band 17, the cylinder portion 18c is pressed into the hole 17h2 to form a hermetic seal therebetween and the holes 18h2 and 17h3 align with each other to allow air from the outside to flow into the box 18 and wise versa. This ensures that air can be supplied to the air pump (not shown) inside the box 18, and wise versa (i.e. when the diaphragm is inflated or deflated after starting or finishing to measure a persons' blood pressure, respectively.

The distance between the square wall 17w and the oval cavity 17c along the length of the band should be the same as the distance between the center of the top of the arm and the radial artery 1 along the bottom of a persons arm, so that when the square wall is positioned on top of the a persons arm (i.e. the way a normal watch is usually worn around a persons arm), the oval cavity 17c is exactly over the radial artery.

The band 17 is injection molded using conventional means which are well know in the art of injection molding. Since the band 17 is made of flexible material, such as silicone, rubber, latex, etc., the band 17 becomes possible to injection mold.

It should be noted that people have different wrists sizes, and, accordingly, the band 17 can be injection molded, for example, in three different length designated as SMALL, MEDIUM AND LARGE, or even more sizes, such as the diameter of a persons wrist, to allow the cavity 17c to always be positioned over the radial artery when the square wall 17w is positioned over the top of a persons' wrist, no matter the size of the wrist. However, regardless of the size of the band (i.e. the length of the band and the distance between the square hole 17w and the cavity 17c), the inner dimensions of the square wall 17w and the inner dimensions of the cavity 17c should always be the same, so that the same size box 18 for housing the electronic blood measuring device 19 as well as the same size diaphragm 12000N can be used in all the different size bands 17. In this way, every person can have a specifically designed band 17 for measuring blood pressure, while maintaining the cost of manufacturing to a minimal (i.e., since the same diaphragm 12000N and the same box 18b can be used with all of the bands 17 regardless of their length's being small, medium or large).

Accordingly, the cost of the band can be made very cheap, the cost of the diaphragm 12000N can be made very cheap as well, since they are both injection molded using conventional injection molding techniques.

Numeral 18t designates three round bottoms which when pressed change the time, date and other functions on the display. In other words, when the electronic blood pressure measuring device is not being used for measuring blood pressure, the display 18d is used to show the present time, date, etc.

It should be noted that preferably, the electronic blood pressure measuring device further comprises;

Storing means (i.e. RAM) for storing a table of information representative of the systolic, diastolic, blood pulse rate as well as the corresponding time and date each of these set of measurements was made;

Information transmitting means for transmitting the information stored in said table of information to a cell telephone (i.e. via blue tooth often available in cell phones), so that this information stored in the electronic blood pressure measuring device can be sent to a hospital via a persons cell phone. In this way, a doctor can constantly monitor a patients condition, while not having to have the patient hospitalized.

Blood Pressure Band with Manual Air Pump

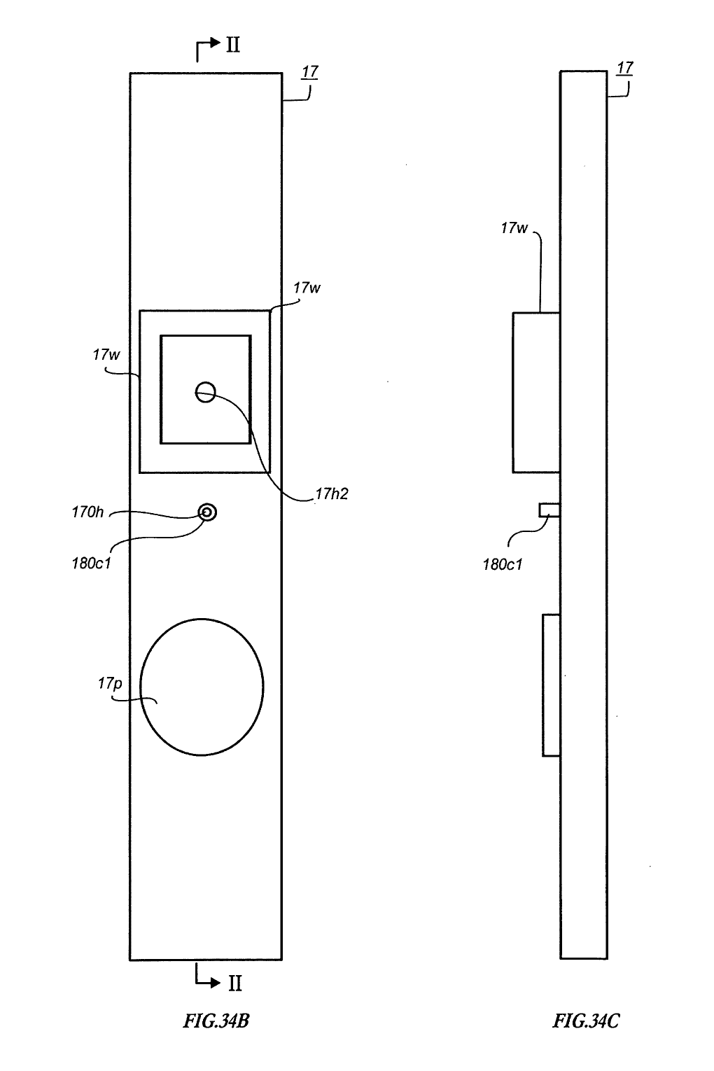

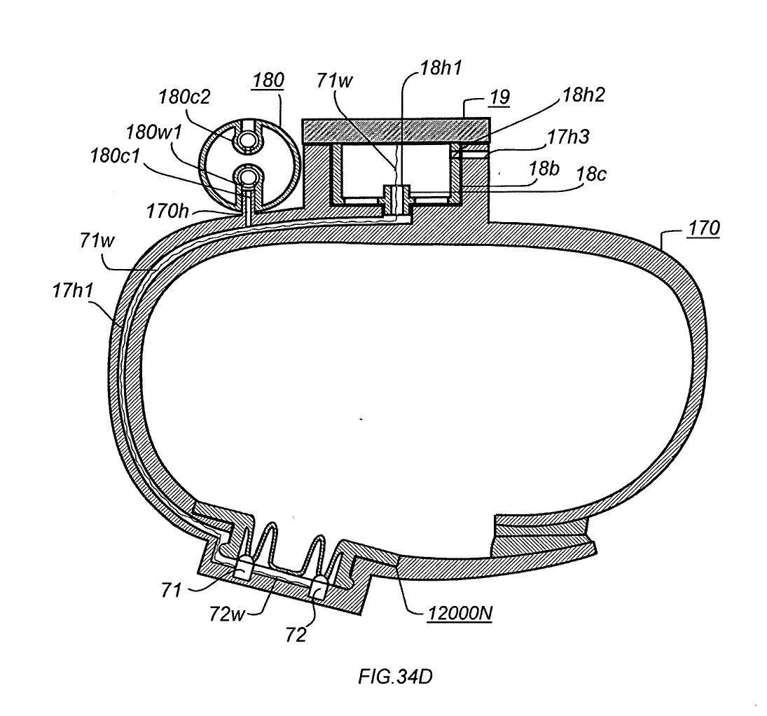

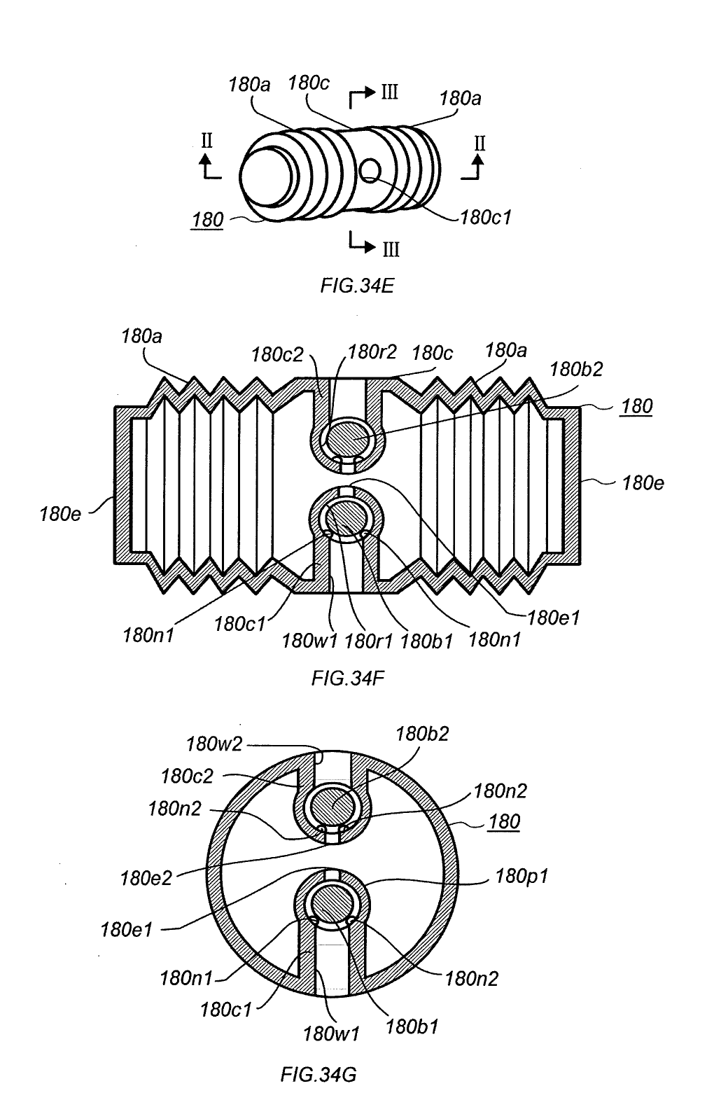

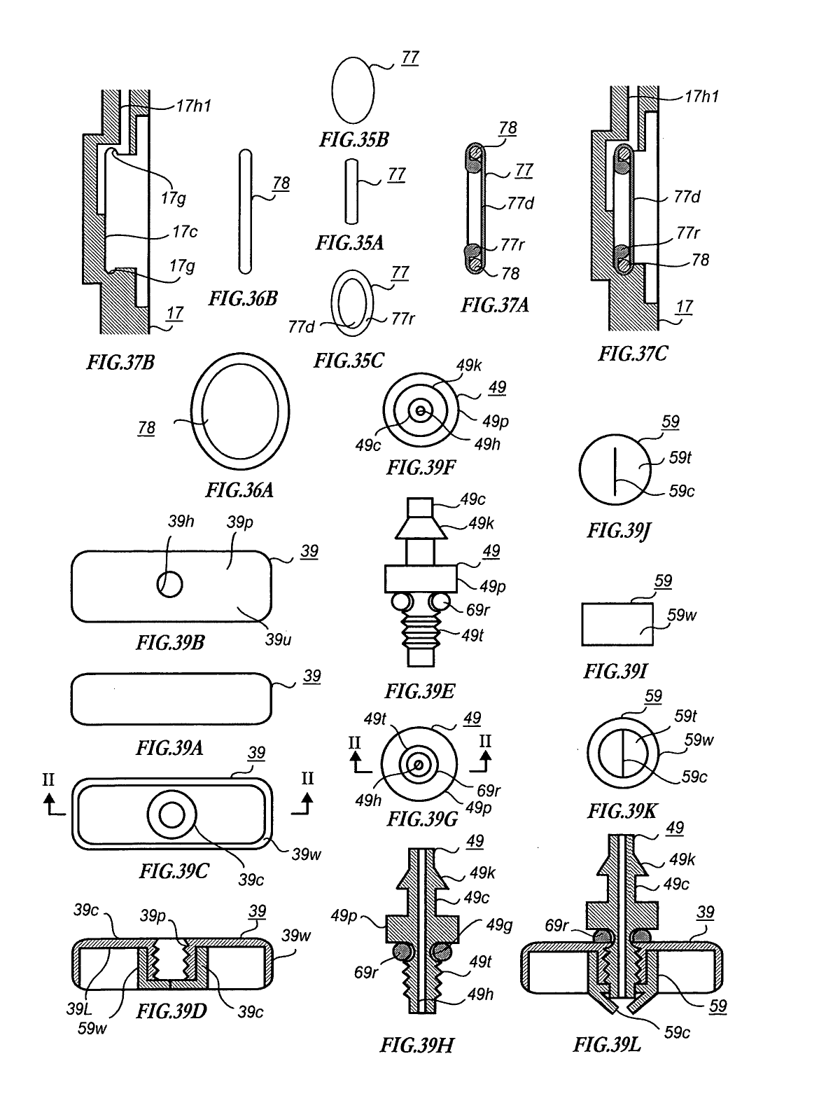

FIG. 34A shows a perspective view of an band 170 having a blood pressure measuring device 18, a diaphragm 12000N and a manual air pressure pump 180 mounted therein according to another embodiment of the present invention.

FIGS. 34B-34D show, a front view, a side view and a side cross sectional view at line II-II in FIG. 34B of a bendable but not stretchable band 170 for a blood pressure measuring device according to another embodiment of the present invention. The band 170 is similar to the band 17 and only the differences therebetween will be described herebelow.

The band 170 further comprises a round cylinder 180c1 integrally formed with the band 170 along the outer surface thereof. The hole 170h inside the cylinder 180c1 and the hole 17h2 inside the band 170 are joined, so that air can freely flow between them. The cylinder 180c1 is formed near the side of the square wall 17w which is nearest to the oval cavity 17c. Accordingly, the cylinder 180c1 is formed on the outer surface of the band 170 and is located between the square wall 17w and the oval cavity 17c.

FIGS. 34E-34G show a perspective view, and side cross sectional views at lines II-II and III-III in FIG. 35E of a manual rubber air pump 180 according to the present invention. Referring to the Figs., the air pump 180 comprises a cylindrical central portion 180c and two cylindrical end portions 180a. Each of the end portions 180e is integrally formed with the central portion 180c on either end of the cylindrical central portion 180c. Numeral 180e designates end walls integrally formed with the cylindrical end portions 180a along the extending ends of the end portions. The outer surface of the cylindrical outer portions 180a is shaped in an accordion like fashion, so that when the end walls 180e are manually pushed towards each other, the cylindrical end portions 180a easily compress and move towards each other (this compressed state will hereafter be referred to as the air pumping state). Then, when the end walls 180e are released, the air pump 180 returns to its original extended shape (hereafter referred to as the air pump air filling state or air filling state).

The accordion portions 180a and the central portion 180c are hollow inside. Numerals 180c1180c2 respectively designate round cylindrical portions each of which is formed in the center of the central portion 180c. The cylindrical portions 180c1, 180c2 each extend inwardly from the outer surface of the central portion 180c in a perpendicular direction with respect to the accordion end portions 180a. Each of the cylindrical portions 180c1 and 180c2 partly extends into the center of the central portion 180c.

Each of the cylindrical portion 180c1, 180c2 has a round protrusion (i.e. round opening 180r1, 180r2 integrally formed therewith along the inner ends thereof, respectively, for receiving a round ball 180b1, 180b2 therein. Since the pump 180 is formed using resilient material, it is easy to form the pump 180 using conventional vacuum injection molding techniques.

The pump 180 is formed of any resilient material such as rubber, silicone, polypropylene, etc. The round balls 180b1, 180b2 may be formed using resilient material such as rubber, silicone, etc. or using hard materials such as plastic using conventional injection molding techniques.

The balls 180b1, 180b2 are identical in size and shape and have an outer diameter which is slightly greater than the inner diameter of the cylindrical inner walls 180wl, 180w2. Further the outer diameter of the balls 180b1, 180b2 is slightly smaller than the inner diameter of the round portions 180rl, 180r2, and accordingly, the balls 180b1, 180b2, can freely move inside the round portions 180rl, 180r2.

The inner facing ends of the round protrusions 180rl, 180r2, have round openings 180e1, 180e2 for allowing air to pass therethrough. The respective outer facing surface and inner facing surface of the round protrusions 180rl, 180r2 each has little bumps 180rl, 180r2 respectively formed along the inner surface thereof for respectively allowing air to pass between the outer surfaces of the balls 180b1, 180b2 and the side walls of the inner and outer round portions 180r1, 180r2. In this way, the round protrusions 180rl, 180r2 and the respective balls 180b1, 180b2 each provide one way air valves for pumping air through the pump 180.

To mount the manual air pump 180 on the band 170, first some adhesive material is applied to the outer surface of the cylindrical portion 180c1 and then the cylindrical portion 180c1 in the band 170 is inserted into the cylindrical hole 180w1 in the cylindrical portion 180c1 in the central portion 180c of the pump 180. The outer diameter of the cylindrical portion 180c1 and the inner diameter of the inner cylindrical surface 180w1 are the same, and accordingly, the two surfaces can be glued to each other using an adhesive material (not shown).

FIG. 34D shows a side cross sectional view of the manual air pump 180 mounted on the band 170. Referring to the Fig., it can be seen that the cylinder 190c1 snugly fits inside the cylindrically shaped hole 180w1 and extends into the hole 180w1 to the point where the round opening 180r1 is formed. Further, that the outer surface of the wall 17w closest to the diaphragm opening 17c is adjacent to the outer wall of the accordion shaped cylindrical portions 180a and the outer wall of the central portion 180c of the air pump 180. Furthermore, that the length of the air pump 180 is substantially the same as the width of the air band 170, and accordingly that the wall 17w nearest the side of the air pump 180 provides a physical barrier for protecting the air pump 180 from accidentally being ripped off.

With this structure, it is very easy for the person wearing this band 170 to use their thumb and index finger of their other hand (i.e. the hand which the band 170 is not on) to push the end walls 180e of the pump 180 towards each other, thereby pumping air into the cavity 17c and inflating the diaphragm 12000N mounted therein.

Namely, when the end walls 180e are pushed towards each other, the ball 180b2 moves up (i.e. due to air flow inside the pump 180) causing the ball 180b2 to block air from flowing out of the inside of the pump 180 through the cylinder portion 180c2. On the other hand, the ball 180b1 gets pushed down inside the cylinder against the bumps 180n1, which allow air to flow around the outer surface of the ball 180b1 into the cylindrical hole 170h2 in the band 170 causing the diaphragm to inflate. Next, when the end walls 180e of the pump 180 are released, the accordion like portions return to their original extended form (due to the elastic nature of the material used in forming the air pump 180) and the opposite of what was described above with respect to the balls 180b1, 180b2 happens, whereby air in the diaphragm 12000 is blocked from flowing back into the pump 180 by the ball 180b1 and air is sucked back into the inner chamber in the pump 180 (i.e. the ball 180b2 allows air to flow around it due to the bumps 180n2) to ready the air pump 180 for the next pumping mode.

With this embodiment, first the START bottom is pushed on the electronic blood pressure measuring device 18, and then the manual air pump is pressed and released several times until the pressure on the display show 180 mmHg. Even though the air pump only pumps a few ml every time it is pressed, the volume of air in the cavity 17c is very small, and accordingly, with less than 10 presses, the desired pressure can be achieved.

By having the manual pump 180, the electronic blood pressure measuring device 170 can be substantially reduced in size, since the manual air pump 180 can be used in parallel to an electric air pump 32 inside the device 18 or the electric pump 32 can be eliminated altogether, whereby a substantial reduction in battery power and size as well as an overall reduction in size of the device can be achieved.

Furthermore, the battery inside the electronic blood measuring device (not shown) would be much smaller and, accordingly, the electronic blood measuring device can be made smaller.

Alternatively, the electronic air pump inside the device 18 can be kept and the user can choose whether to pump up the diaphragm 12000N manually or electronically.

The present invention is not intended to be limited to the manual air pump described above and any conventional air pump may be used without departing from the scope and spirit of the present invention.

Pre-Stretched Diaphragm

FIG. 35A-35D show a side view, a front view, a back view and a side cross sectional view at line II-II in FIG. 35C of a pre-stretch diaphragm 77 according to another embodiment of the present invention. The diaphragm 77 comprises an outer oval shaped ring portion 77r and a flat diaphragm portion 77d integrally formed therewith along the central portion thereof. The diaphragm portion 77d is much thinner than the ring portion 77r.

The diaphragm 77 is formed using conventional injection molding techniques from rubber, latex, etc.

FIG. 36A and FIG. 36B show a front view and a side view of an oval shaped ring 78.

The outer diameter and the shape of the ring 78 is the same as the inner diameter and shape of the oval groove 17g in the band 17 or 170, so that the oval ring 78 snugly fits therein. The oval shaped diaphragm 77 is smaller than (about half size) the outer diameter of the oval ring 78.

To mount the diaphragm 77 on the ring 78, the ring portion 77r of the diaphragm 77 is manually pulled around the outer circumference of the ring 78.

FIG. 37A shows a side cross sectional view of the diaphragm 77 mounted on the ring 78.

FIG. 37B shows a side cross sectional partial view of the band 17 where the cavity 17c is formed with no diaphragm inserted therein.

FIG. 37C shows a side cross sectional view of the diaphragm 77 mounted on the ring 78 which are then together mounted inside the oval groove 17g in the cavity 17C in the band 17.

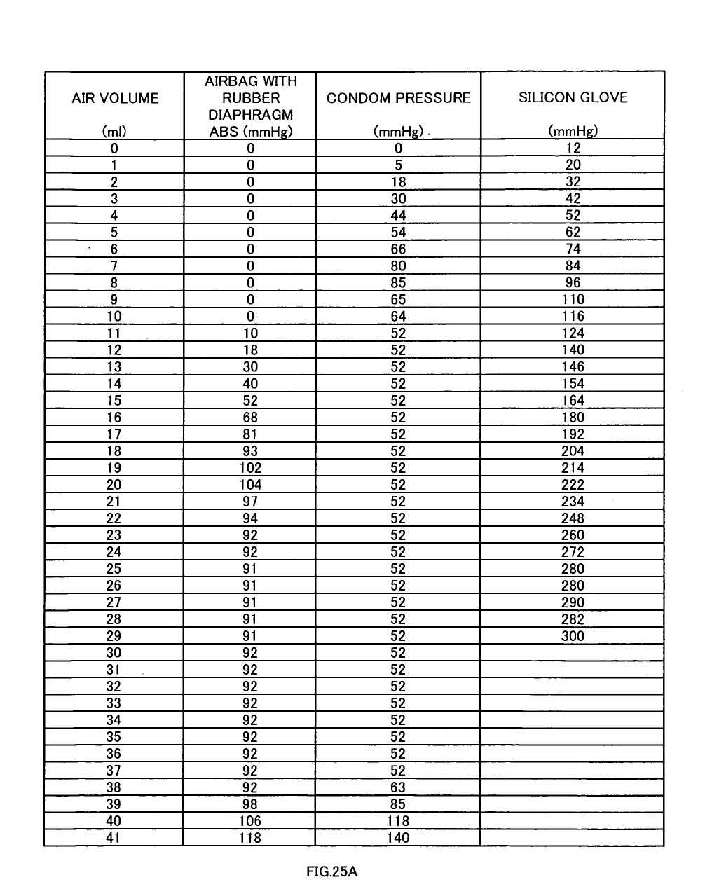

Accordingly, with this diaphragm 77 and ring 78, the diaphragm 77 can be pre-stretched to a point just past the section C in the diagram shown in FIG. 25B, and, accordingly, the diaphragm 77 exhibits only the flat characteristics shown by section D in FIG. 25B. With this embodiment, a very simple algorithm can be used to calculate the AABPOA. Furthermore, since the diaphragm 77 is pre-stretched, it is also much thinner than before stretching, and, accordingly, will further enhance the sensitivity and fidelity the diaphragm exhibits to detecting blood pressure and blood pulses.

It should be noted that the thickness of the diaphragm portion 77d of the diaphragm 77 can be varied. Namely,the central portion of the diaphragm portion 77d can be made thinner along the central portion thereof and the thickness of the diaphragm portion 77d can be increased along the outer portion thereof (i.e. nearest to the ring portion 77r), so that the central portion of the diaphragm 77d stretches out first towards the artery 1 as the diaphragm portion 77d is filled with air while the outer portion of the diaphragm portion 77d only stretches out at higher air pressures, thereby further enhancing the DLEBM characteristics of the diaphragm 77.

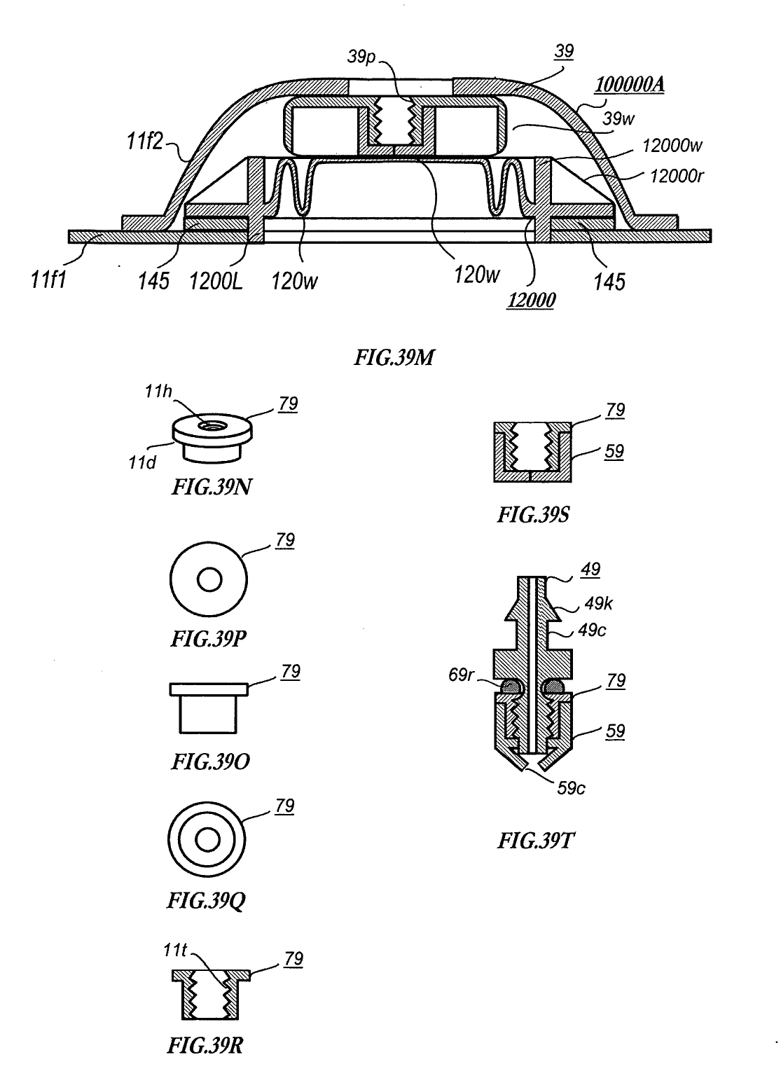

FIGS. 39A-39D show a side view, a top view, a bottom view and a cross sectional view at line II-II in FIG. 39C of a nipple 39 of for an air bag according to another embodiment of the present invention.

FIGS. 39E-39H show a side view, a top view, a bottom view and a cross sectional view at line II-II in FIG. 39G of a connector 49 for connecting and disconnecting an air hose 14 to and from the nipple 39 according to another embodiment of the present invention.

FIGS. 39I-39K show a side view, a top view and a bottom view of a rubber cap 59 for blocking water from entering through the nipple.

FIG. 39L shows a side cross sectional view of the connector 49 mounted in the nipple 59.

Referring to FIGS. 39A-39D, the nipple 39 comprises a rectangular shaped plate 39p and having downwardly facing side walls 39w integrally formed therewith along the perimeter of the plate 39p. Numeral 39h designates a round hole formed through the center of the plate 39p.

Numeral 39c designates a round cylindrical portion formed on the lower surface 39L of the plate 39p. Numeral 39t designates a female thread formed on the inner surface of the cylindrical portion 39c. The length of the cylindrical portion 39c is less than the length of the side walls 39w.

The nipple 39 is made from hard plastic and the size of the plate 39p is ⅔ the size of the film 11f2.

The rubber cap 59 comprises a round top portion 59t and side walls 59w. The cap 59 can is formed using conventional injection molding techniques.

Numeral 59c designates a straight cut formed by a knife in the center of the top portion 59t.

The cap 59 frictionally fits on the cylindrical portion 39c of the nipple 39.

The connector 49 comprises a cylindrical portion 49c having a cone shaped protrusion 49k formed on the outer upper surface thereof, a thread portion 49t formed on the lower end thereof and a round protrusion 49p integrally formed on the cylindrical portion 49c along the center of the cylindrical portion. Numeral 69r designates a round rubber O ring mounted in an round groove 49g formed in the outer surface of the cylindrical portion 49.

FIG. 39M shows the nipple 39 mounted in an air bag 100000A according to another embodiment of the present invention. The air bag 100000A is the same as the air bag 100000 shown in FIG. 11K, with the exception of changing the nipple 11000 with the nipple 39. All the other parts are the same and the manufacturing steps of the air bag 100000A are the same as the air bag 100000 as described above.

FIG. 39N-39R show a perspective view, a side view, a top view, a bottom view and a cross sectional view at line II-II of FIG. 39P of a nipple 79 according to another embodiment of the present invention;

FIG. 39S shows a cross sectional view of the nipple 79 shown in FIG. 39P having the rubber cap 59 mounted thereon;

FIG. 39T shows a cross sectional view of the nipple 79 shown in FIG. 39P having the rubber cap 59 mounted thereon and the connector 49 mounted therein.

Referring to FIG. 39K-39Q, the nipple 79 comprises a round disc 11d having a smooth upper surface 11U and lower surface 11L. Numeral 11h designates a through hole formed at the center of the disc 11d, the through hole 11h extending from the upper surface 11U to the lower surface 11L of the disc 11d. Numeral 11t designates a thread formed on the inner surface of the through hole 11h.

FIG. 39P shows a side cross sectional view of the connector 49 and the rubber cap 59 mounted in the nipple 79 Accordingly, the connector 49 can be connectively disconnected to the nipple 79 in the same way as described above with respect to the nipple 39.

The nipple 79 is mounted in any of the airbags disclosed in this application using double sided tape or any other conventional means.

Flow Chart

FIG. 25A shows a table of the measured air pressure inside an air bag (i.e. hereinafter referred to as MAP) as a function of the volume of air inside the air bag (hereinafter referred to as AV). These values where actually derived from the air bag 100000 shown in FIG. 11, but instead of the diaphragm 120000, the diaphragm 12 shown in FIGS. 5A-5B was used

Normally, just using the diaphragm 12 would not yield accurate results in measuring blood pressures. The reason for this is due to the fact that with the disposable air band of the present invention there is no way of knowing exactly how tight or loose a patient mounts the air band around their wrist. If the air band is wound around the wrist very tightly, then less air would be required inside the air bag to determine the systolic and diastolic blood pressures. this case, less air in the air bag means less stretching of the rubber diaphragm 12 and accordingly less force to stretch the rubber diaphragm 12. In other words, the tighter the air band is wound around a patients wrist, the more accurate the systolic and diastolic readings would be. The reason for this is that presently there is no way of distinguishing between how much of the air pressure inside the air bag is required to stretch the diaphragm 12 and how much air pressure inside the air bag is actually exerted on the radial artery 1 (hereafter referred to as AABPOA). However, it is very uncomfortable to constantly wear the air band tightly around a persons wrist and, accordingly, it is very desirable to be able to wear the air band loosely around the wrist and still be able to determine the correct systolic and diastolic blood pressures.

Furthermore, in case the clip 13 and the hose 14 (shown in FIG. 15) are used to join the air bag 31 to the electronic blood pressure measuring device, it was experimentally noted that the air hose also expanded and contracted as the pressure therein increased and decreased, and that the air in the air hose 14 increased in a direct proportion to the air pressure in the air hose. Accordingly, to achieve the most accurate systolic and diastolic blood pressures, it would be necessary to include experimental measurements of the air bag air pressure as a function of the air bag air volume (hereinafter referred to as ABAP/ABAV characteristics or air bag stretching characteristics, hereinafter referred to as ABS characteristics) when the air bag is not pressed against the arm.

Furthermore, it was experimentally noted that the air hose 14 used to connect the clip 13 to the electronic blood pressure measuring device also expanded as the air pressure therein increased from 0 mmHG to 300 mmHg, and accordingly, measurements of the air hose air pressure vs the air hose air volume (hereinafter referred to as AHAP/AHAV or air hose stretching characteristics AHS characteristics) should be taken into consideration in determining the systolic and diastolic blood pressures.

The air hose used was made of silicone and had a 3 mm inner diameter, a 5 mm outer diameter and was 400 mm long. The silicone hose used is very flexible, so that it is very easy and comfortable to handle and even when the air hose is moved around, it does not interfere with or disturb the position of the clip and/or air bag.

This objective is achieved by using the following embodiment and associated flow chart.

FIG. 25B shows a graph representative of the rubber diaphragm ABS characteristics values in the table of FIG. 25A

Referring to the table in FIG. 25A, and the corresponding graph in FIG. 25B, it can be seen that from 0 ml to 10 ml the air pressure required is 0 (hereinafter designated as air volume area A). Then, when the air bag is full and the rubber diaphragm starts expanding (i.e. stretching) outwards of the air bag, the pressure increases linearly from 0 mmHg to 93 mmHg as the air in the air bag increases from 10 ml to 18 ml (hereinafter referred to as linear area B) and then the air pressure increases further to 104 mmHg as the air volume is increased from 20 ml (hereinafter referred to as non linear area C) and then as the air volume increases to from 25 ml to 36 ml, the pressure stays constant at 93 mlHg (hereinafter referred to as linear area D). Then, the air pressure goes up exponentially as the rubber reaches the limit of its elasticity where it finally breaks (hereinafter called area E). Accordingly, an object of the present invention is to have the rubber diaphragm inflated only in the A and B areas, i.e. the linear areas, where the ABS stretching characteristics of the rubber are stable and predictable.

The electronic blood pressure measuring device according to the present invention comprises:

an air bag;

an air pump;

means for measuring the air volume being pumped into the air bag;

MAP means for measuring the air pressure inside the air bag;

ABS characteristics storing means for storing a table of air pressures inside the air bag as a function of the air in said air bag when the air bag is not subjected to any external forces;

AABPOA calculating means for determining the actual air pressure applied by the air bag on the artery as a function of air volume in the air bag;

means for determining the systolic/diastolic blood pressure as a function of the AABPOA.

In one embodiment of the present invention, said AABPOA determining means comprises:

Means for measuring the volume of air in said air bag:

Means for measuring the air pressure in the air bag (hereinafter referred to as MAP) as a function of the volume of air in said air bag;

an air bag stretching characteristics table (hereinafter referred to as ABS) representative of the air pressure in the air bag (hereinafter referred to as ABAP) as a function of the volume of air in said air bag (i.e. ABAV) while said air bag is not applied to a persons arm; and

subtracting means for subtracting said respective values of ABS pressures from respective MAP pressures as a function of respective quantities of air in said air bag (i.e. AVAB).

Another embodiment of the present invention, instead of said chart, said ABS chart comprises an a mathematical algorithm for determining the ABS pressures as a function of air volume in said air bag.

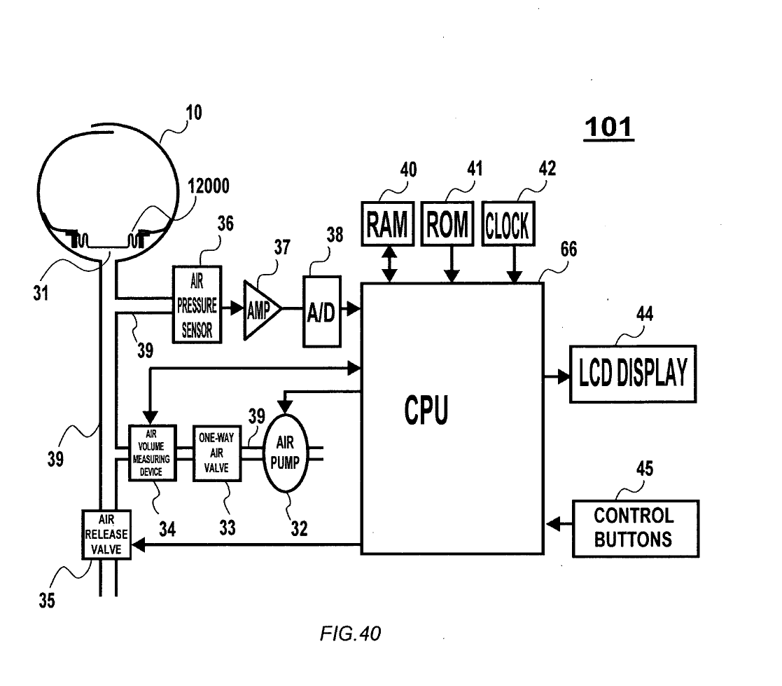

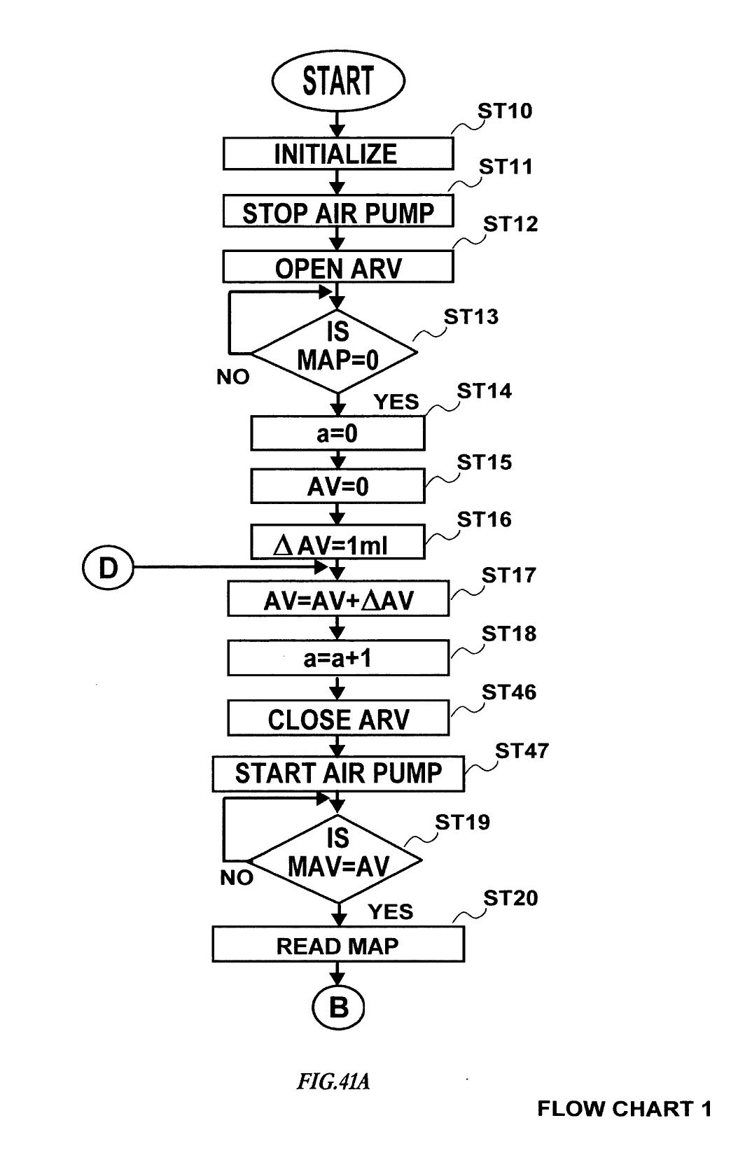

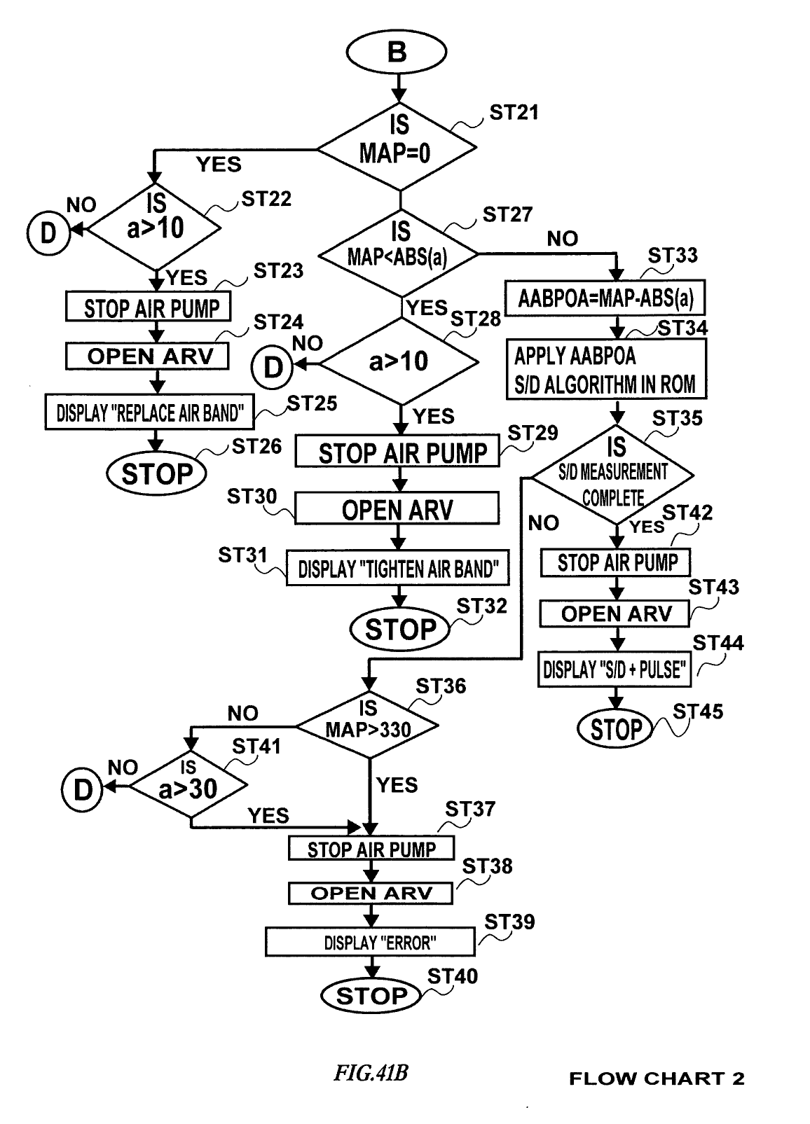

FIG. 40 shows a block diagram of an electronic blood pressure measuring device according to the present invention.

The electronic blood pressure measuring device comprises:

ABS storing means for storing the air bag stretching pressure as a function of the air volume in the air bag;

AABPOA calculating means for calculating the actual air bag pressure on the artery as a function of the air volume in said air bag;

means for calculating the systolic and diastolic blood pressure as a function of said AABPOA; and

means for displaying the thus calculated systolic and diastolic blood pressures.

The AABPOA determining means comprises:

means for measuring the volume of air inside an air bag;

means for measuring the air pressure (hereinafter referred to as MAP) inside the air bag as a function of the air volume inside the air bag;

air bag stretching characteristics storing means for storing data representative of the air pressure required to blow up the air bag as a function of the volume of air in said air bag (i.e. hereinafter referred to as the ABS characteristics)

subtracting means for subtracting said AABPOA from said MAP as a function of air volume in said air bag;

means for storing a conventional algorithm for determining the systolic and diastolic blood pressures based on said calculated AABPOA and the shape of the blood pressure pulse provided by a pressure sensor as a function of time.

Referring to FIG. 40, numeral 10 generally designates an air band having an air bag 31 integrally formed therewith, numeral 32 designates an air pump, numeral 33 a one way air valve for allowing pressurized air to only flow from the air pump 32 to an air volume measuring device 34 (hereinafter referred to as an AVMD 34). Numeral 35 designates an air release valve (hereinafter referred to as an ARV 35), and numeral 36 an air pressure sensor.

Numeral 39 designates an air passage way which connects the air bag 31, the pressure sensor 36, the ARV 35, the AVMD 34, the one way air valve 33 and the air pump 32 to each other for allowing air to flow therebetween.

Numeral 37 designates an amplifier for amplifying the signal from the air pressure sensor 36, numeral 38 designates an analog to digital converter for converting the analog signal from the amplifier 37 to a digital signal, numeral 66 designates a central processing unit (hereinafter referred to as CPU 46) numeral 40 designates a random access memory (hereinafter referred to as RAM 40) numeral 41 designates a read only memory (hereinafter referred to as ROM 41) for storing data representative of the ABS stretching characteristics of the air bag 31 (i.e. the information in table 25A) as well as flow charts for calculating the actual air bag pressure on the artery (hereinafter referred to as AABPOA) as will be described hereinafter. Numeral 42 designates a clock, numeral 45 designates the control buttons such as START, STOP, etc., and numeral 44 designates a display for displaying the systolic and diastolic blood pressure as well as the blood pulse rate. The RAM 40 is used to temporarily store air volume and measured air pressures (hereinafter referred to as MAP) provided by the A/D converter 38.

FIG. 41A, 41B show a FLOW CHART 1 for determining the systolic and diastolic blood pressures as a function of the air bag ABS characteristics and actual (real) air bag pressure on the radial artery (hereinafter referred to as the AABPOA) by the air bag 31 according to present invention.

Referring to FIGS. 41A and 41B, step ST10 designates an initialization step for resetting the clock to 0, and the registers to 0. Next, at step ST11, the CPU 40 instructs the air pump 32 to stop (i.e. just in case it is ON when the START button 45 is manually pressed). Next, at step ST12 the ARV 35 is instructed to OPEN to allow any air left in the air bag 31 from a previous BP measurement to flow out of the air bag 31. Next, at step ST13, it is repeatedly checked if the measured air pressure (MAP) is equal to 0 (i.e. if all the air in the air bag 31 is out of the air bag 31). When it is determined that all the air in the air bag 31 is out of the air bag 31, the program proceeds to step ST14 where a register is set to 0 (i.e. a=0). Next, at step ST15 a register AV is set to 0 (i.e. AV=0). The register AV represent the amount of air to be pumped into the air bag 31. Next, at step ST16, a register DAV is set at 1 (representative of 1 ml. of air). Steps ST10 to step ST16 are initialization steps which are only executed once during the systolic and diastolic blood pressure calculating program (i.e. FLOW CHART 1 AND FLOW CHART 2).

Next, at step ST17, the present air volume AV is increased by the incremental air volume DAV (i.e. AV=AV+DAV). Next, at step 18, the value in the register a is increased by 1 (i.e. a=a+1). Next, at step ST46, the CPU 66 instructs the ARV 35 to close and then at step ST47 instructs the air pump 32 to be turned ON. Next, at step ST19 it is repeatedly determined if the measured air volume by the AVMD 34 is equal to the value in the AV register (MAV<AV). If the answer is NO, step ST19 is repeatedly executed. If the answer is YES (i.e. implying that the measured air volume (hereinafter also referred to as MAV) by the air volume measuring device 34 (hereinafter also referred to as AVMD 34) in the air bag 31 is equal to the air value in the register AV), the program proceed to step ST20 where the measured air pressure MAP inside the air bag 31 (i.e. air pressure measured by the pressure sensor 36) is read. Next, at step ST21, it is determined whether or not the measured air pressure is equal to 0. If the answer is YES, the program proceeds to step ST22. At step ST22 it is determined whether or not the value in the register a is greater than 10. If at step ST22 the answer is NO, the program returns to step ST17, where the value in the AV register is increased by 1 ml (i.e. AV=AV+DAV).

If the answer at step ST22 is yes, (implying that there must be an air leak in the air bag 31, since if there was no air leak, the measured air pressure MAP should be greater than 0 according to the ABS characteristics table shown in FIG. 25A), the program proceeds to step ST23 where the air pump 32 is instructed to STOP. Next, at step ST24, the ARV 35 is opened and then at step ST25 the message “FAULTY AIR BAG” is displayed on the display 44.

On the other hand, if the answer at step ST21 is NO, (i.e. the MAP by the pressure sensor 36 is not 0), the program proceeds to step ST27, where it determined whether or not the measured air pressure MAP inside the air bag is less than or equal to the air pressure required to stretch the air bag for the air volume presently in the air bag (i.e. MAP<ABS(a)). If the answer is YES, the program proceeds to step ST28 where it is determined whether or not the value in the register “a” is greater than 10. If the answer at step ST28 is NO, the program returns to step ST17, where the AV is increase to AV+DAV. If at step ST28 the answer is YES, the program proceeds to step ST29 where the CPU 40 instructs the air pump 32 to STOP. Then, at step ST30, the CPU 40 instructs the air release valve ARV 35 to open. Next, at step ST31, the CPU instructs the display 44 to display “TIGHTEN THE AIR BAND”. Next at step ST32, the program is STOPPED.

If at step ST27 the answer is NO, the program proceeds to step ST33, where the AABPOA is calculated by subtracting the ABS(a) from the presently measured MAP. Next, at step ST34, the presently calculated AABPOA in step ST33 as well as the output from the A/D converter 38 (i.e. a digital signal coming from the air pressure sensor 36 is provided to a conventional systolic/diastolic calculating algorithm pre-stored in the ROM 41). Next, at step ST35,it is determined whether or not the systolic/diastolic algorithm in the ROM 41 has finished calculating the systolic/diastolic blood pressures from the data thus far provided.

If at step ST35 the answer is NO, the program proceeds to step ST36 where it is determined weather or not the MAP is greater than 330 mmHg? If the answer is YES, the program proceeds to step ST37 where the CPU instructs the air pump to STOP. Next, at step ST38, the ARV35 is instructed to OPEN. Next, at step ST39, the display unit 44 is instructed to display “ERROR” and then at step ST40 the program is instructed to STOP.

If at step ST36 the answer is NO, the program proceeds to step ST41, where it is determined whether or not “a” is greater than 30. If the answer is YES the program proceeds to step ST37 previously described. If the answer at step ST41 is NO, the program returns to step ST17, where the value of AV is increase by the increment DAV.

If at step ST35 the answer is YES, the program proceeds to step ST42 where the air pump is instructed to stop. Next, at step 43 the ARV35 is instructed to open. Next, at step ST44, the display 44 is instructed to display the systolic and diastolic values calculated by the systolic/diastolic algorithm stored in the ROM 41 as well as the blood pulse rate. Next, at step ST45. the program is instructed to STOP.

It should be noted that the present invention is not intended to be limited to the above described embodiments, and that numerous variations are possible without departing from the scope and spirit of the present invention.

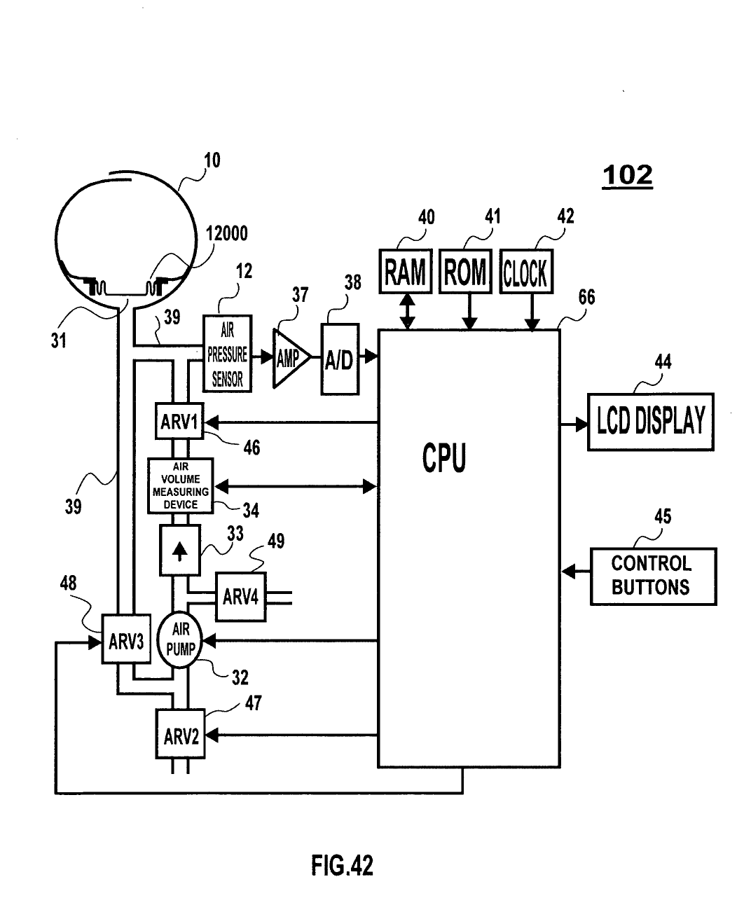

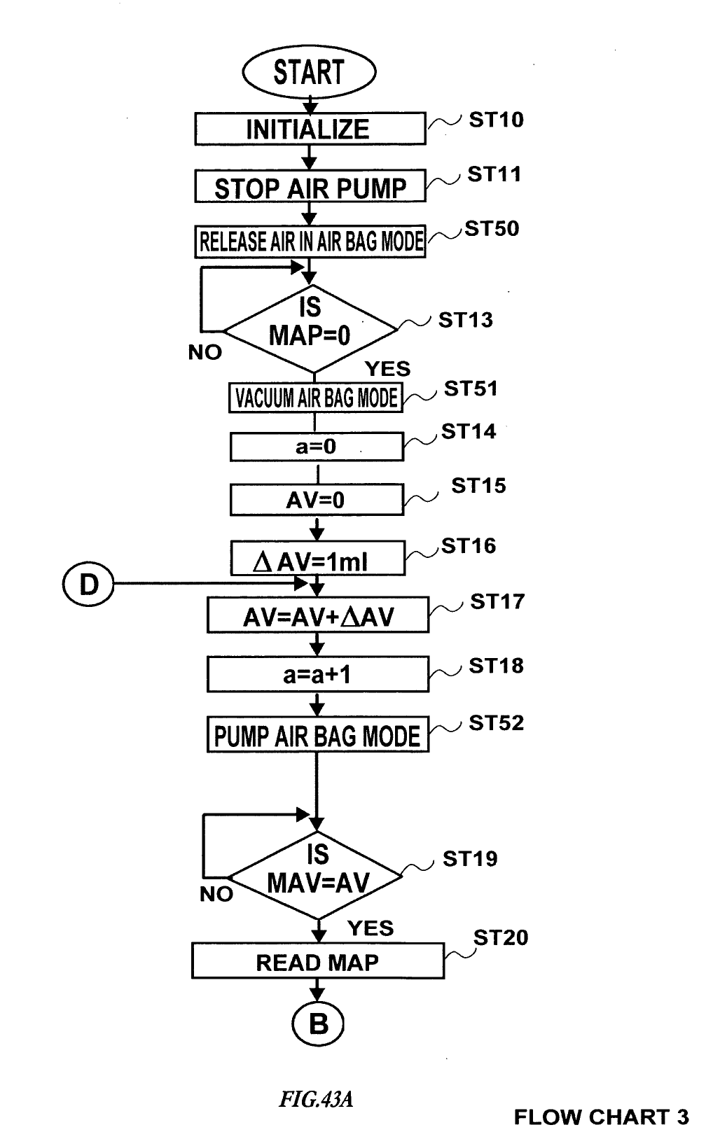

FIG. 42 shows a block diagram of an electronic blood pressure measuring device 102 according to another embodiment of the present invention. The electronic blood pressure measuring device 101 did not ensure that the initial air volume before starting to pump air into the air bag 12 was 0. With this embodiment of the device 102, it is ensured that the blood pressure measuring device 102 sucks out all the air out of the air bag 31 first, before proceeding to do ABAV measurements with the air volume measuring device 34.

The electronic blood pressure measuring device 102 comprises:

ABS storing means for storing the air bag stretching characteristics as a function of the air volume in the air bag;

an AABPOA calculating means for calculating the actual air bag pressure on the artery as a function of the air volume in said air bag;

means for calculating the systolic and diastolic blood pressure as a function of said AABPOA; and

means for displaying the thus calculated systolic and diastolic blood pressures.

The AABPOA determining means comprises:

means for measuring the volume of air inside an air bag;

means for measuring the air pressure (hereinafter referred to as MAP) inside the air bag as a function of the air volume inside the air bag;

air bag stretching characteristics storing means for storing data representative of the air pressure required to blow up the air bag as a function of the volume of air in said air bag (i.e. hereinafter referred to as the ABS characteristics)

subtracting means for subtracting the respective ASB pressure from the respective MAP as a function of air volume in said air bag;

means for storing a conventional algorithm for determining the systolic and diastolic blood pressures based on said calculated AABPOA and the shape of the blood pressure pulse provided by a pressure sensor as a function of time.

Wherein, said means for measuring the volume of air inside an air bag comprises the steps of

1. vacuuming all the air out of said air bag;

2. pumping air into said air bag; and

3. measuring the volume of air in said air bag while air is being pumped into said air bag.

Referring to FIG. 42, the electronic blood pressure measuring device 102 is very similar to the electronic blood pressure measuring device 101 and only the differences therebetween will be described herebelow.

The device 102 comprises four air release valves ARV46, ARV47, ARV48 and ARV49 instead of one ARV 35. These valves, enable:

1. air to be pumped into the air bag 31;

2. air to be released out of from the air bag 31; and

3. air to be vacuumed out of the air bag 31.

The ARV46, 47, 48 and 49 are normally in the open state (hereinafter referred to N/O state). In the N/O state, no electricity is applied to the ARVs, and accordingly, require no electricity most of the time, and accordingly, saves energy.

To pump air into the air bag 31, ARV1 and ARV2 are OPEN and ARV3 and ARV4 are closed. In this state, when the air pump 32 is switched ON, the air flows through the ARV2, the air pump 32, the one way valve 33, the AVMD 34, the ARV1 and into the air bag 31.

To release air from the air bag 31, ARV2 and ARV3 are OPEN while ARV1 and ARV4 are CLOSED or OPEN (either state will work). In this state, air flows out of the air bag 31 through the ARV3 and ARV2.

To vacuum air out of the air bag 31, ARV3 and ARV4 are OPEN, while ARV2 and ARV1 are CLOSED. In this state, when the air pump 32 is turned ON, air flows through the ARV3, the air pump 32 and ARV4, thereby sucking any air left in the air bag 31 out of the air bag 31.

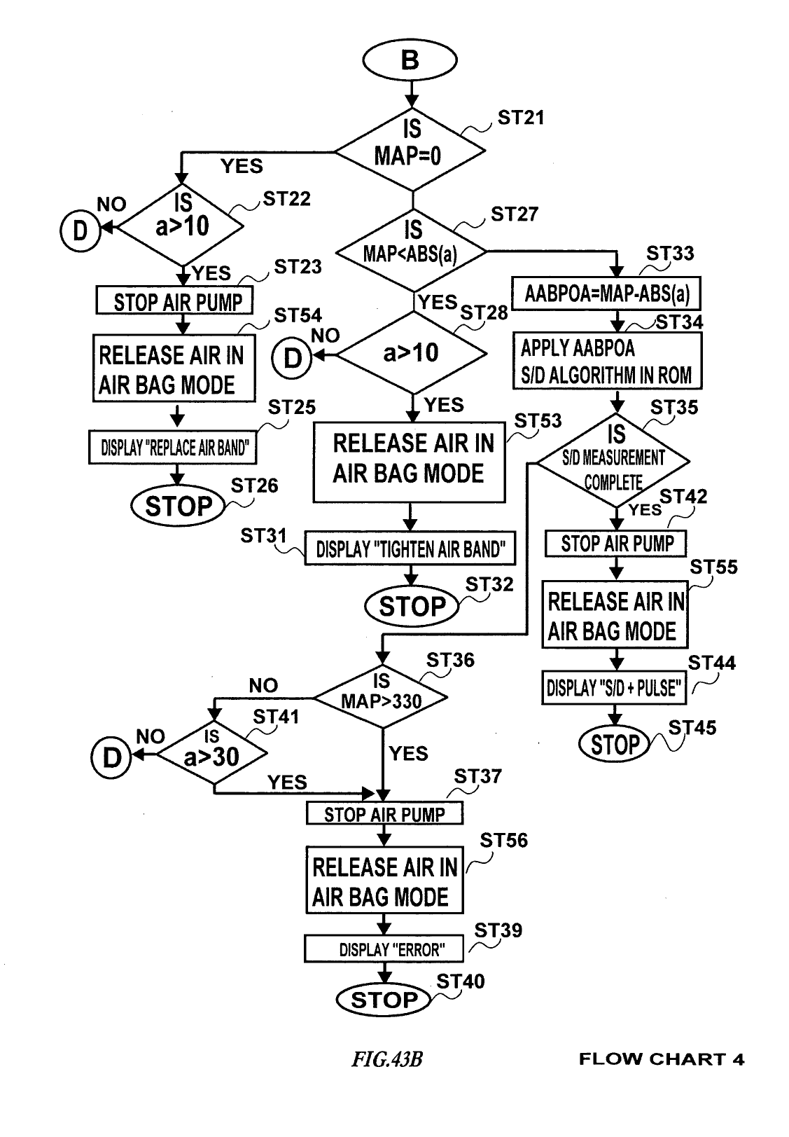

FIG. 43A and 43B show another embodiment of a FLOW CHART 3 and FLOW CHART 4 for determining the systolic and diastolic blood pressures according to the present invention. The FLOW CHART 3-4 is similar to the FLOW CHART 1-2 and only the differences therebetween will be describe herebelow.

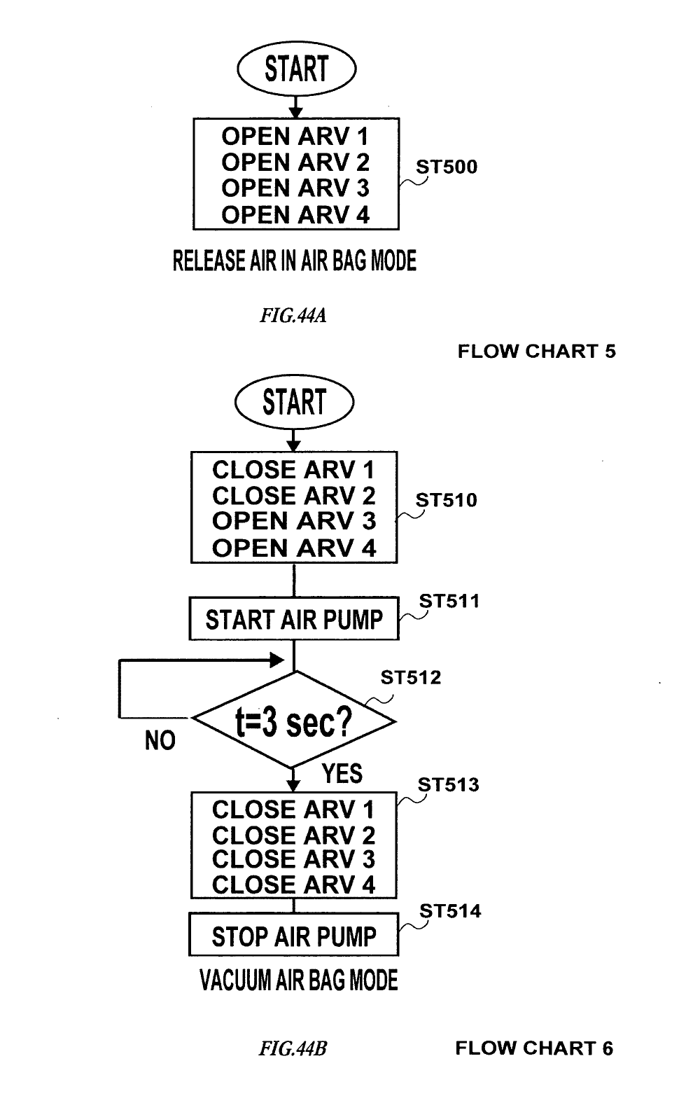

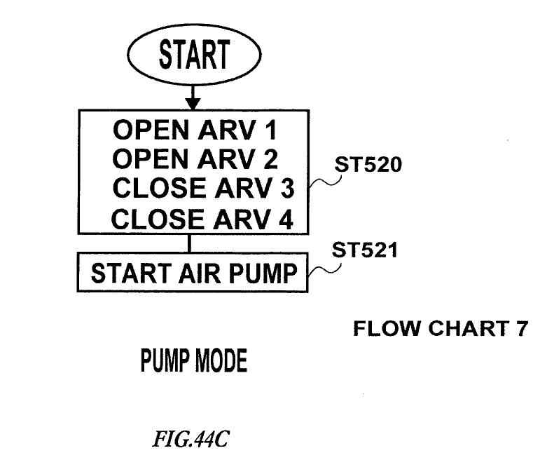

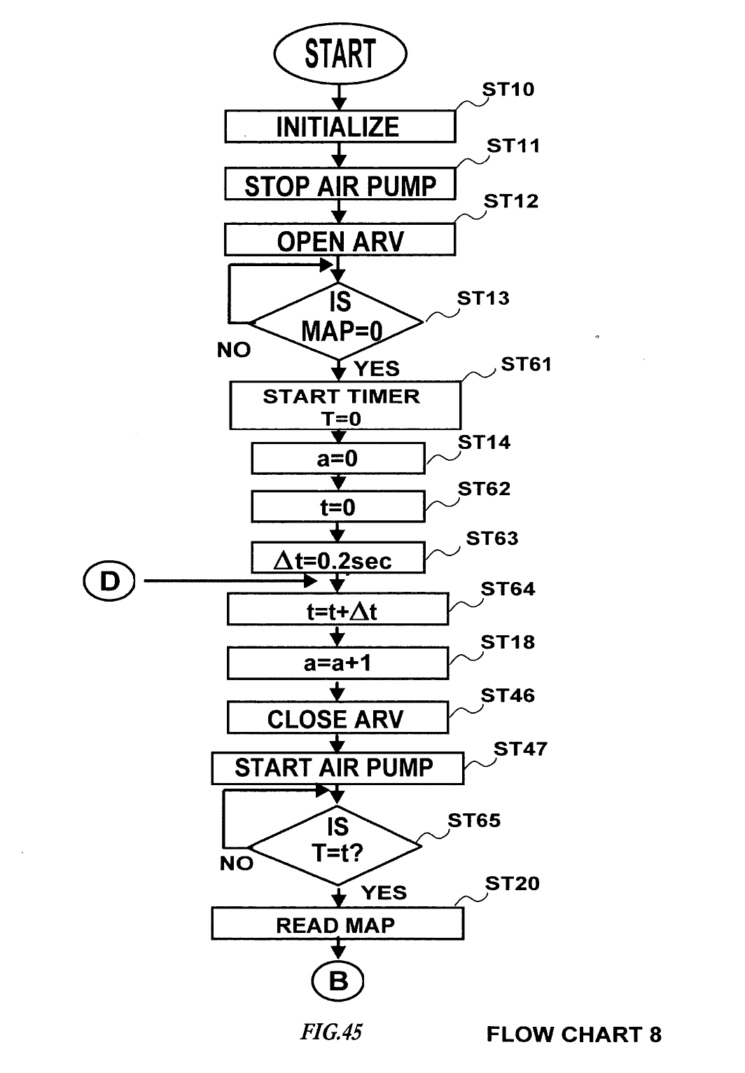

FIGS. 44A, 44B and 44C show subroutines for “RELEASE AIR IN THE AIR BAG MODE”, “VACUUM AIR BAG MODE” and “PUMP MODE” of operation.

Referring to FIGS. 43A, 43B, 44A, 44B and 44C, it can be seen that after step ST11, at step ST50, the device 102 instructs the “RELEASE AIR IN AIR BAG MODE of operations. In this state, the subroutine shown in FLOW CHART 5 is carried out. Referring to the FLOW CHART 5, the CPU 66 instructs the ARV1, ARV2, ARV3 and ARV4 to stay OPEN (which is their normal state requiring to electricity), thereby providing an air flow pass through the ARV3 and ARV2.

Next, between steps ST13 and step ST14 an additional step ST51 is inserted. Step ST51 is a subroutine shown in FLOW CHART 6, where the VACUUM AIR BAG MODE of operation is executed, namely, the CPU 66 instructs the device 102 to remove all the air out of the air bag 31. This step is necessary to ensure that no residual air remains in the air bag, so that the starting initial volume of air in the air bag 31 is always the same. Referring to the FLOW CHART 6, at step ST510 the ARV1 and ARV2 are instructed to CLOSE while ARV3 and ARV4 are instructed to OPEN. Next, at step 511, the air pump 32 is instructed to start pumping. Next, at step 512 it is determined whether or not a time of 3 seconds has passed from the time the air pump was instructed to start pumping. The step ST512 is constantly executed until it is determined that 3 seconds have elapsed at which time the program proceeds to step ST513, where ARV1, ARV2, ARV3 and ARV4 are instructed to CLOSE. Next, at step ST514, the air pump 32 is instructed to STOP.

Next, the program returns to step ST14 where the register “a” is set to 0 (i.e. a=0). Accordingly, with this embodiment it is possible to vacuum the air out of the air bag 31 ensuring that the starting point of measuring air volume in the air bag 31 is always the same.

Between steps ST18 and ST19, the device is instructed to start the “PUMP AIR BAG MODE” of operation. As shown in FLOW CHART 7, at step ST520, the ARV1 and ARV2 are instructed to OPEN, while ARV3 and ARV4 are instructed to CLOSE. Next, at step ST521, the air pump is instructed to turn ON and start pumping. The rest of the program should be obvious to those familiar in the art.

Flow Chart 8

Since the pumping characteristics (i.e. specifications) of air pumps are well documented, the air pump 32 can be chosen to be one that has a constant rate of pumping, for example 5 ml/second. Since the air bags of this invention require about 30 ml of air to be full, it would take about 6 seconds to fill the air bag which is an acceptable length of time to determine the systolic and diastolic blood pressures.

Accordingly, by choosing an air pump for air pump 32 which has a constant air pumping rate, it is possible to determine the amount of air in the air bag as a function of the time the pump is pumping. In this way, the air volume measuring device 34 can be eliminated, thereby, reducing the cost and the size of the electronic blood pressure measuring devices 101 or 102.

FIG. 45 shows FLOW CHART 8 for measuring the systolic and diastolic blood pressure while not requiring the air volume measuring device 34; The FLOW CHART 8 is similar to the FLOW CHART 1 and only the differences therebetween will be described herebelow.

Referring to FLOW CHART 8, if at step ST13, the answer is YES, the program proceeds to step ST61, where a timer (i.e. clock 42) is reset to 0. Next, at step ST14 the register “a” is set to 0. (i.e. a=0). Next, at step ST62, a register “t” is set to 0 (i.e. t=0). Next at step ST63, a register Dt is set to 0.2 seconds (i.e. Dt=0.2). Next at step ST64, the value in the register “t” is increased by 0.2 seconds (i.e. t=t+Dt). Next, the program proceeds to step ST18, where the value in register “a” is increased by 1 (i.e. a=a+1). Next, at step ST46, the air release valve 35 is instructed to CLOSE. Next at step ST47, the air pump 32 is instructed to start pumping. Next at step ST65, it is determined whether or not the time in the clock 42 has reached the time in the register “t” (i.e. T=t?) If the answer is NO, the step ST65 is repeated until it is determined that T=t, at which time the program proceeds to step ST20 which was described above with respect to FLOW CHART 1. The rest of the flow chart is identical to that of FLOW CHART 1.

Accordingly, with this FLOW CHART 8, since the air pump pumping rate is 5 ml per second and since the Dt is set at 0.2 seconds, the amount of air pumped by the air pump every second is 1 ml.(i.e. 5 ml/sec×0.2=sec 1 ml). This value of 1 ml coincides with the DAV=1 ml increment changes set at step ST16. in FLOW CHART 1, and, accordingly, the same results in calculating the systolic and diastolic blood pressures can be achieved.

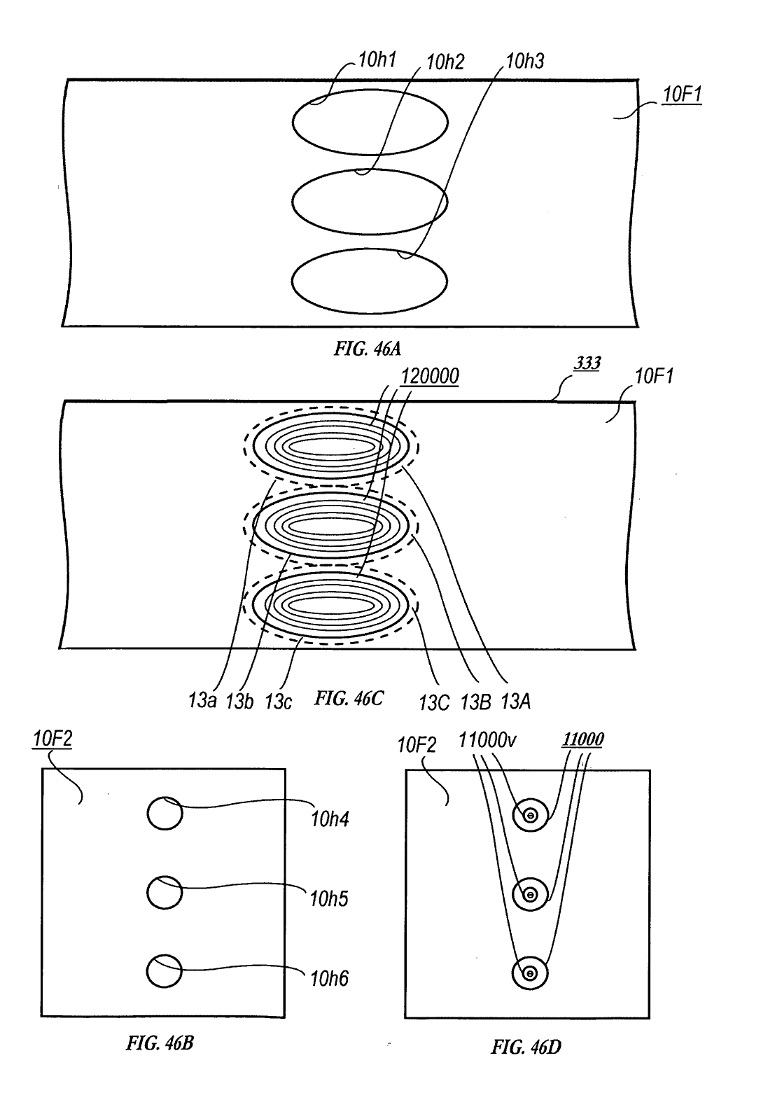

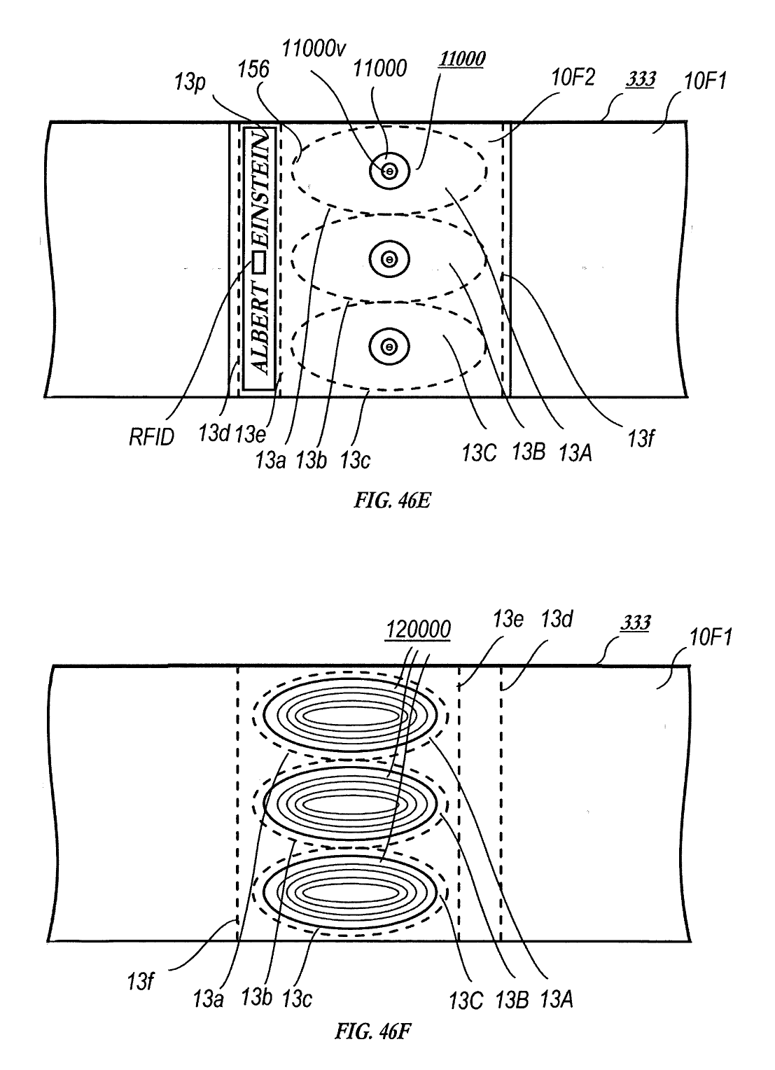

FIGS. 46A-46F show the parts and the steps to manufacture a multi-air-bag-band 333 according to another embodiment of the present invention. The multi-air-bag-band 333 is very similar to the air band 100000 shown in FIGS. 11K and 11L and the only difference is that instead of having only one air bag 100000, there are three identical air bags 13A, 13B and 13C integrally formed with each other. With this embodiment, much more information can be retrieved regarding the physical condition (i.e. physical state) of the patient. Namely, the speed of the blood pulse through the artery, the hardness of the artery, etc. Furthermore, a more accurate systolic and diastolic measurement can be achieved. Still further, the results measured in the three air bags 13A, 13B and 13C can be compared to each other and if the results measured by the three air bags 13A, 13B and 13C do not correlate with each other in real time, the measurement is stopped and an ERROR message is displayed, thereby providing for more accurate and reliable measurements.

FIG. 46A shows a front view of a first film 10F1. The first film 10F1 is long enough to go around a persons arm (i.e. about 30 cm.) and wide enough to support three diaphragms 120000 in parallel with each other along the width of the film 10F1. The first film 10F1 has three oval holes 10h1, 0h2 and 10h3 punched therethrough. Each hole 10h1, 0h2 and 10h3 is provided for supporting a diaphragm 120000 therein (i.e. similarly to the way the diaphragms 120000 are mounted on the film 11f1 of air bag 100000 shown in FIGS. 11A-11J). The oval holes 10h1, 10h2 and 10h3 are directly next to each other in the width direction of the film 10F1, with the narrower sides of the diaphragms 120000 facing the width direction of the film 10F1 and the longer sides of the diaphragm 120000 facing the long direction of the film 10F1. The shape and size of the holes 10h1, 10h2 and 10h3 are the same as the shape and size of the outer dimensions of the lip 12000L of the diaphragm 120000.

FIG. 46B shows a front view of a second film 10F2. The second film 10F2 is slightly longer than the length of the diaphragm 12000 and has the same width as the film 10F1. The film 10F2 has three holes 10h4, 10h5 and 10h6 punched therethrough. The holes 10h4, 10h5 and 10h6 are punched in a straight line in the width direction of the film 10F2, so that when the film 10F2 is placed on top of the film 10F1, each of the holes 10h4, 10h5 and 10h6 lies directly above each of the holes 10h1, 10h2 and 10h3, in the film 10F1, respectively. Each of the holes 10h4, 10h5 and 10h6 is provided for supporting a nipple 11000 therein (i.e. similarly to the way the nipples 11000 are mounted on the film 11f2 of air bag 100000 shown in FIGS. 11A-11J). The size of each of the holes 10h4, 10h5 and 10h6 is the same as the size of the outer diameter of the head portion 11000p of the nipple 11000.

The films 10F1 and 10F2 are made of bendable but non-stretchable film such as polyethylene, etc.

FIG. 46C shows a front view of the first film 10F1 having three diaphragms 120000, respectively mounted in a respective holes 10h1, 10h2 and 10h3 either using DST as described above with respect to the air bag 100000 shown in FIGS. 11K and 11L, or by using glue, etc.

FIG. 46D shows a front view of the film 10F2 having three nipples 11000 respectively mounted in a respective holes 10h4, 10h5 and 10h6 either using DST as described above with respect to the air bag 100000 shown in FIGS. 11H and 11I, or by using glue, heat sealing, etc.

FIG. 46E shows a top view of the multi-air-bag-band 333. Referring to the Fig., the second film 11F2 (having the three nipples 11000 mounted therein) is mounted on the first film 11F1 (having the three diaphragms 120000 mounted therein). Next, the films 11F2 and 11F1 are heat sealed along dash lines 13a, 13b and 13b, thereby forming three oval shaped air bags 13A, 13B and 13C, each of the air bags 13A, 13B and 13C having one of the nipple 11000 and one of the diaphragms 120000 mounted therein. Each of the heat seals 13a, 13b and 13b are formed just outside the periphery of each of the diaphragms 120000, respectively.

Numerals 13d and 13e designate two perpendicular heat seals formed at a distance of about 10 mm along one end of the film 11F2 in the width direction thereof. Numeral 13f designates a perpendicular heat seal formed along the other end of the film 11F2 in the width direction thereof and joins the films 11F1 and 11F2 to each other.

The heat seals 13d and 13e seal the films 11F1 and 11F2 to each other and create a small pocket between the films 11F1 and 11F2 into which a strip of paper 13p having the name ALBER EINSTEIN printed thereon is inserted. The strip of paper 13p also has a RFID (i.e. radio frequency identification device or a bar code reader) mounted thereon along a central portion thereof. Accordingly, when a clip 133 is mounted on the nipples 11000 in the multi-air-bag 333, a radio frequency reader 130000RF (or bar code reader) mounted in the clip 133 which will be described herebelow can transmit the patients I.D. to a electronic blood pressure measuring device 103 which will also be described herebelow.

FIG. 46F shows a bottom view of the multi-air-bag-band 333. Referring to the Fig., the first film 11F1, having the three diaphragms 120000 mounted therein is heat sealed along heat seals 13a-13f to the second film 11F2, the film 11F2 having the three nipples 11000 mounted therein.

The first and second films 10F1 and 10F2 are made from bendable but not stretchable material such as polyethylene, etc., and preferably should be transparent.

Referring to FIGS. 46A-46F, numerals 13a, 13b and 13c each designate an oval heat seal which hermetically seals the first and second films 10F1 and 10F2 to each other, thereby forming three air bags 13A, 13B and 13C, respectively. Each of the three air bags 13A, 13B and 13C has one nipple 11000 and one diaphragm 120000, each nipple 11000 being directly above a respective diaphragm 120000. The thus formed three air bags 13A, 13B and 13C provide a multi-air-bag-band 333.

Accordingly, when the multi-air-bag-band 333 is wound around a patients wrist, the three air bags 13A, 13B and 13C can be positioned directly above the artery 1 along the length of the artery 1. More specifically, the three air bags 13A, 13B and 13C would not only be directly over the artery 1 but also air bags 13A would be closest to the heart, air bag 13B would be next closest to the heart and air bag 13C would be furthest from the heart.

Since the air bags 13A, 13B and 13C can be filled and emptied individually, much more information regarding the physical condition of the patient can be obtained.

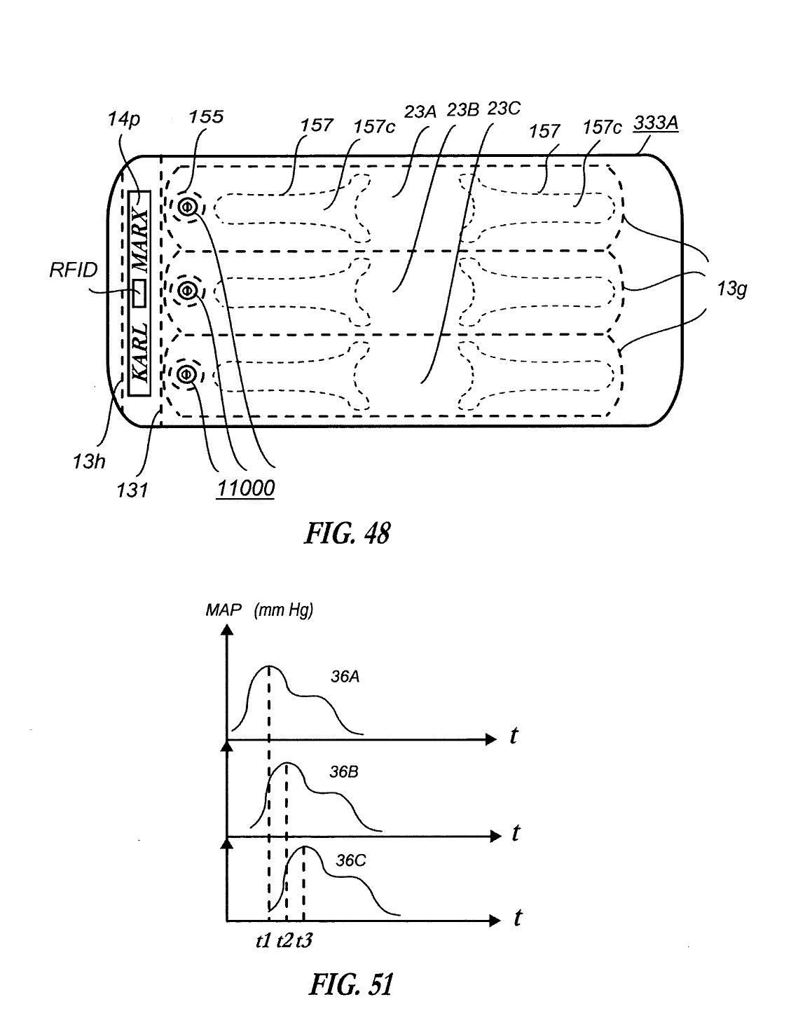

FIG. 48 shows a front view of a multi-air-bag-band 333A according to another embodiment of the present invention. The multi-air-bag-band 333A is very similar to the air band 1000B shown in FIG. 28F and the only difference therebetween will be described herebelow.

Referring to FIG. 48, the multi-air bag-band 333A comprises an upper and a lower bendable and stretchable films f4 which are wide enough to manufacture three identical air bags 23A, 23B and 23C therein. Each of the three air bags 23A, 23B and 23C is identical to the air band 1000B shown in FIG. 28F.

Numerals 13g (shown by dash lines) designate oval shaped heat welds formed around each of the air bags 23A, 23B and 23C, thereby separating the air flowing in air bags 23A, 23B and 23C while allowing the air bags 23A, 23B and 23C to be physically attached to each other. The air bags 23A, 23B and 23C of the air band 333A are identical to each other and also identical to the air band 1000B shown in FIG. 28F.

Numerals 13h and 13i designate two perpendicular heat seals formed at a distance of about 10 mm from each other. The heat seals 13h and 13i seal the films f4 to each other and create a small pocket between the films between the films f4 into which a strip of paper 14p having the name KARL MARX printed thereon is inserted. The strip of paper 14p also has a RFID (i.e. radio frequency identification device or a bar code reader) mounted thereon along a central portion thereof. Accordingly, when a clip 133 is mounted on the nipples 11000 in the multi-air-bag band 333A, a radio frequency reader 130000RF (or bar code reader) mounted in the clip 133 which will be described herebelow can transmit the patients I.D. to a electronic blood pressure measuring device 103 which will also be described herebelow.

FIG. 49A shows a perspective view of a multi-clip 133 according to the present invention.

FIGS. 49B-49D show a bottom view, a back view and a front view of the multi clip 133.

The clip 133 is very similar to the clip 13000 shown in FIG. 20A-20H and only the differences therebetween will be described herebelow.

Referring to FIG. 49A-49C, the multi-clip 133 comprises three clips 133A, 133B and 133C which are integrally formed with each other along the respective sides thereof. Each of the clips 133A, 133B and 133C are identical in size and structure to the clip 1300O shown in FIGS. 20A-20H.

Accordingly, when the back ends 13D, 13E of the multi-clip 133 are pressed towards each other, the front ends 13A, 13B of the multi-clips 133 move away from each other and wise versa.

The distance between the three round shafts 13000c in the multi-clip 133 is the same as the distance between the three round cylindrical holes 11000w in the three nipples 11000 mounted in the multi-air-bag-band 333 or the multi-air-bag-band 333A. Accordingly, when the multi-clip 133 is mounted on the head portions 11000p of the nipples 11000, the shaft portions 11000c of the nipples 11000 slide into the slots 130s in the multi clip 133 until the each of the shaft portions 11000c of each of the nipples 11000 buts up against the back surface 130e of the slots 130s, at which time the three round shaft portions 13000c of the three clips 133A, 133B and 133C of the multi-clip 133 are perfectly aligned with the three round cylindrical holes 11000w in the three nipples 11000 mounted in the multi-air-bag-band 333A, thereby allowing for the easy mounting of the multi-clip 133 on the multi-air bag-band 333 or the multi-air-bag-band 104.

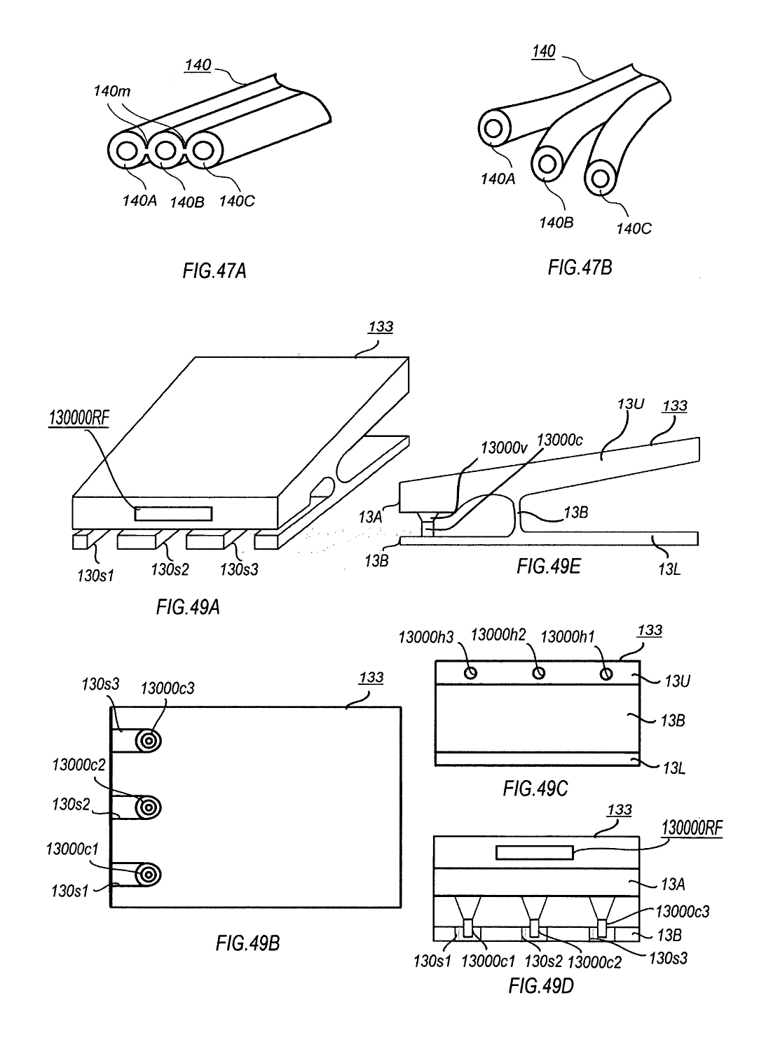

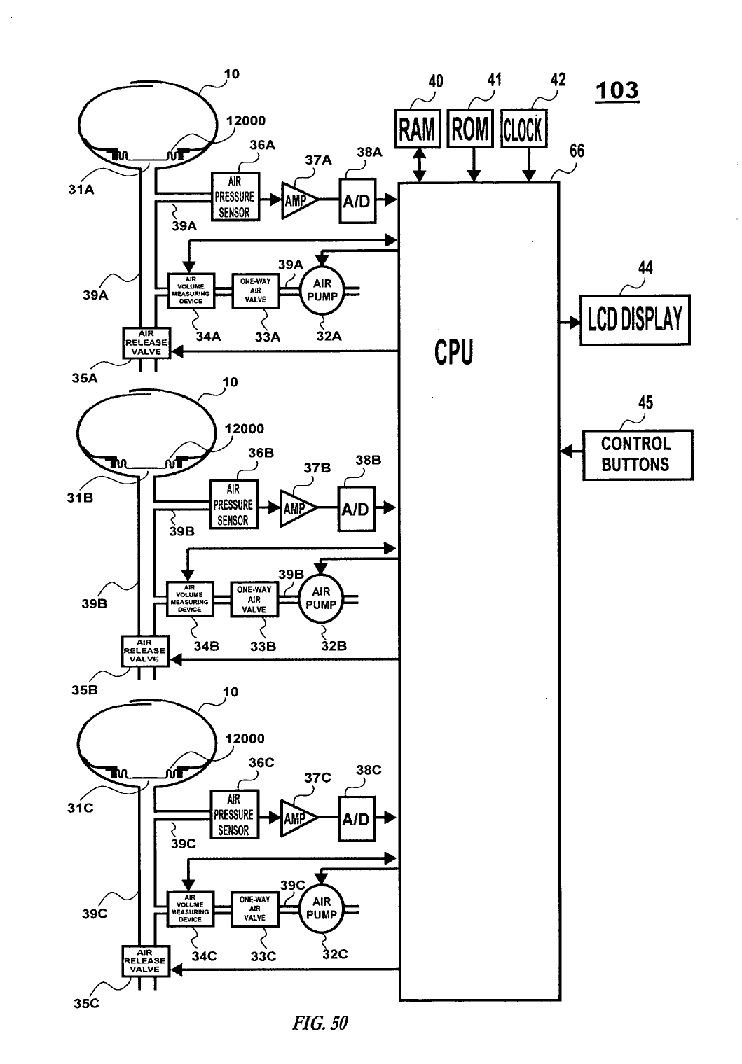

FIG. 47A, 47B show perspective views of an air hose 140 comprising three air hoses 140A, 140B and 140C integrally formed with each other. Referring to FIGS. 47A and 47B, numeral 140 generally designates a three air hoses 140A, 140B and 140C which are attached to each other by a thin membrane 140m along the respective sides thereof. The air hose 140 is made of flexible silicone. The hoses 140A, 140B and 140C can be easily separated from each other by ripping or cutting the membranes 140m therebetween (i.e. as shown in FIG. 47B). The outer diameters of the air hoses 140A, 140B and 140C are the same or slightly bigger than the air holes 13000h3 formed in the back ends of the upper arms 13U of the multi-clip 133, so that the extending ends of each of the hoses 140A, 140B and 140C can be frictionally inserted into a respective hole 13000h3 in the multi-clip 133. The other ends of the air hoses are similarly inserted into a respective cylindrical hole 15h1 of one of three plastic coupling devices 15 (identical to the one shown in FIG. 21K). The respective other holes 15h3, 15h4 of each of the three plastic coupling devices 15 is hermetically coupled to a respective one of three air pump and one of three air pressure sensors, as will be described herebelow with respect to FIG. 50.

FIG. 50 shows a block diagram of a multi-air-bag electronic blood pressure measuring device 103 according to another embodiment of the present invention. The multi-air-bag blood pressure measuring device 103 (hereinafter referred to as the multi-air-bag-device 103) is very similar to the blood pressure measuring device 101 and only the differences therebetween will be described herebelow. Similar parts will be designated by the same numerals.

Referring to FIG. 50, it can be seen that all the parts designated 31 to 39 in FIG. 40 have been re-designated as parts 31A-39A and that the parts 31A-39A provide the same functions as the respective parts 31-39, respectively. Furthermore, that the parts 31B-39B and the parts 31C-39C are respectively identical to the respective parts 31A and 39A. Furthermore that the parts 31A-39A, 31B-39B and 31C-39C are connected to each other in the same manner as the parts 31-39 are connected to each other as shown in FIG. 40. In other words, the multi-air-bag-device 103, comprises three identical air bags 31A, 31B and 31C, three identical air pumps 32A, 32B and 32C, three identical one way air valves 33A, 33B and 33C, three identical air valve measuring devices 34A, 34B and 34C, three identical air release valves 35A, 35B and 35C, three identical air pressure sensors 36A, 36B and 36C, three identical amplifiers 37A, 37B and 37C, three identical AID converters 38A, 38B and 38C and three identical air passage ways 39A, 39B and 39C as well as the parts mentioned above with respect the device 101 or 102.

Accordingly, with the multi-air-bag-device 103, each of the air bags 13A, 13B and 13C can be inflated and deflated independently from each other in response to respective commands issued by the CPU 66. The ROM 41 contains pre-stored programs and algorithms for determining the systolic and diastolic blood pressures by separately controlling the inflation and deflation of each of the three air bags 31A, 31B and 31C as a function of time. Furthermore, the rate of flow of blood as well as the physical state of the arteries can be determined by the shape of the air pulses in the air bags 31A, 31B and 31C.

FIG. 51 shows a graph of air pressure (MAP) in the air bags 31A, 31B and 31C as a function of time when the air bags 31A, 31B and 31C are filled with air to a point where the largest MAP is provided by each of the three air bags 31A, 31B and 31C. At this time the artery 1 is pressed down by the partially filled air bags 31A, 31B and 31C to a point where the largest MAP air pressure signal is obtained. (i.e. as measured by each of the respective air pressure sensors 36A, 36B and 36C and. as shown in FIG. 50), respectively, the outputs of said air pressure sensors 36A, 36B and 36C are respectively connected to A/D converters 38A, 38B and 38C which then provide in digital form the respective values of measured air pressures MAP in the three air bags of the multi-air bag band 333 or 333A to the CPU 66. As can be seen from the graph 51, the pressure pulse measured by the air pressure sensor 36C legs behind the pressure pulse measured by the air pressure sensor 36B and that the pressure pulse measured by the air pressure sensor 36B legs behind the pressure pulse measured by the air pressure sensor 36A (i.e. as indicated by times t3, t2 and t1, respectively in the graph shown in FIG. 51). Accordingly, with this information, it is very easy to determine the rate of blood flow in the artery 1.

Furthermore, to more accurately measure the systolic and diastolic blood pressures, the three air bags 36A, 36B and 36C can be simultaneously inflated to a point where the greatest amplitude air pulse is detected by the three respective air pressure sensors 36A, 36B and 36C, and then the air bag 13A (i.e., the one closest to the heart) is inflated until no blood pulse is detected by the air bags 13B and 13C.

The multi-air-bag-device 103 can be provided with additional air release valves 35 in a configuration that would allow each of the air bags 31A, 31B and 31C to be filled and emptied individually while only using one air pump 32 to do so.

The air bags 31A, 31B and 31C in FIG. 50 should be replaced with numerals 13A, 13B and 13C in case the multi-air-bag 333 is being used, or by numerals 23A, 23B and 23C in case the multi-air-bag 333A is being used.

What is claimed is:

1. A diaphragm for an air bag for measuring blood pressure which comprises:
a central portion; and
at least one wave portion integrally formed with said central portion along the periphery said central portion.

2. A diaphragm as defined in claim 1, wherein:
said central portion is thinner than the thickness of said wave portion.

3. A diaphragm as defined in claim 1, wherein:
the thickness of said diaphragm is thinnest along a central area of said central portion.

4. A diaphragm as defined in claim 1, wherein:
the thickness of said diaphragm is thinnest along a central area of said central portion and gradually increases in thickness from said central area of said central portion outwards towards said wave portion.

5. A diaphragm as defined in claim 1, wherein:
the thickness of said diaphragm varies along the surface thereof, the thickness of said diaphragm gradually increasing from a central area in said central portion towards said wave portion, so that when an air bag in which said diaphragm is mounted in is inflated with air, said central area in said central portion expands outwardly first followed by the central portion around said central area followed by the unfurling of said wave portion, so that said wave portion not only allows said central portion to easily move outwards of said air bag, but also prevents said central portion from escaping laterally along a patients' arm when said central portion is pressed against an artery in said arm.

6. A diaphragm as defined in claim 1, wherein:
said at least one wave portion comprises a plurality of concentric waves formed around each other, so that when an air bag in which said diaphragm is mounted in is inflated with air, said central portion expands outwardly first followed by the unfurling of said concentric waves.

7. A diaphragm as defined in claim 1, wherein:
said diaphragm is formed of an elastic material.

8. A diaphragm as defined in claim 1, wherein
said diaphragm is formed of rubber.

9. A diaphragm as defined in claim 1, wherein
said diaphragm is formed of latex.

10. A diaphragm as defined in claim 1, wherein
said diaphragm is formed of silicone.

11. A diaphragm as defined in claim 1, wherein:
said central portion and said wave portion are oval in shape, the longer side of said oval central portion being longer than the distance between the radius bone and the digital tendon in a persons writs.

12. A diaphragm as defined in claim 1, wherein:
said central portion and said wave portion are substantially rectangular in shape, the longer side of said rectangular central portion being longer than the distance between the radius bone and the digital tendon in a persons wrist.

13. A diaphragm as defined in claim 1, wherein:
said wave portion is 5 mm high and has a pitch of 1-5 mm.

14. A diaphragm as defined in claim 1, wherein:
the thickness of the thinnest part of said central portion is 0.03 mm.

15. A diaphragm as defined in claim 1, wherein said diaphragm further comprises:
an outer portion integrally formed with said diaphragm along the outer side of said wave portion.

16. A diaphragm as defined in claim 1, wherein said diaphragm further comprises:
an outer portion integrally formed with said diaphragm along the outer side of said wave portion; and
a wall portion integrally formed with said outer portion on an inner surface of said outer portion,
said outer portion being provided for mounting said diaphragm in an air bag.

17. A diaphragm as defined in claim 1, wherein said diaphragm further comprises:
an outer portion integrally formed with said diaphragm along the outer side of said wave portion;
a wall portion integrally formed with said outer portion on an inner surface of said outer portion; and
a lip portion integrally formed with said wall portion on an outer surface thereof for frictionally supporting said diaphragm in a groove formed in a band.

18. A diaphragm as defined in claim 6, wherein:
said central portion and said concentric waves are oval in shape, the longer side of said oval central portion being longer than the distance between the radius bone and the digital tendon in a persons wrist.

19. A diaphragm as defined in claim 1, wherein:
said diaphragm is formed using conventional injection molding techniques.

20. An air bag for measuring blood pressure which comprises:
an inner film;
an outer film, said outer film having a first hole formed therein; and
a first nipple mounted in said outer film, said nipple having a through hole formed therein for allowing air to pass therethrough, said holes being aligned with each other,
said films being hermetically heat sealed to each other, so that when air is pumped through said nipple, said first and second films form at least one air bag, said air bag being long enough to traverse the distance between the radius and the digital tendon in a persons wrist.

21. An air bag as defined in claim 20, wherein:
said films are heat sealed to each other in a pattern, so that when air is pumped through said nipple, said films form a central air bag along the central portion thereof and at least one side air bag on either side of said central air bag, said side air bags being smaller in diameter than said central air bag when said air bags are inflated.

22. An air bag as defined in claim 20, wherein:
said hole in said outer film is formed near one end of said outer film, so that when said air bag is inflated, said nipple is located on one side of said air bag.

23. An air bag as defined in claim 20, wherein:
said films are formed of bendable and stretchable material.

24. An air bag as defined in claim 23, wherein:
said films are long enough to go around a persons wrist.

25. An air bag as defined in claim 20, wherein, said air bag further comprises:
a strap, said strap being long enough to go around a persons wrist, said strap having a hole formed through a central portion thereof,
said strap being mounted over said outer film with said nipple passing through said hole in said strap,
said strap being formed of a bendable but not stretchable material and said inner and outer films being formed of a bendable and stretchable material.

26. An air bag as defined in claim 25, wherein said air bag further comprises:
a third film having a hole formed through the center thereof; and
a fourth film;
said inner film having a round hole formed through the center thereof;
said outer film, inner film and said third and fourth film having the same size and shape and being long enough to traverse the distance between the radius bone and the digital tendon in a persons writs,
said inner film and said third film being heat sealed around the holes formed therein to each along the peripheries thereof,
said third film and said fourth film being heat sealed to each other along the peripheries thereof,
whereby said outer film, said inner film, and said third and fourth film together form a double decker air bag, so that more slack can be taken up by said double decker air bag, when said strap is loosely fitted around a persons wrist.

27. An air bag as defined in claim 20, wherein:
said inner film comprises a plurality of semi spherically shaped bubbles formed along the surface thereof.

28. An air bag as defined in claim 26, wherein:
said inner film comprises a plurality of semi spherical shaped bubbles formed along the surface thereof.

29. An air bag as defined in claim 20, wherein said air bag further comprises:
a second and third nipple and said outer film comprises a second and third hole, said first, second and third nipples being respectively mounted in said first, second and third holes formed in said outer film,
said inner and outer films being heat sealed to each other
, so that when air is pumped through said first nipple
a first set of air bags is inflated,
when air is pumped through said second nipple a second set of air bags is inflated, and
when air is pumped through said third nipple a third set of air bags is inflated,
said air bags being long enough to traverse the distance between the radius and the digital tendon in a persons wrist.

30. An air bag as defined in claim 20 wherein:
said nipple comprises:
a shaft portion, said shaft portion having a through hole formed through the center thereof for allowing air to pass therethrough,
one end of said shaft being mounted on an inner surface of said outer film with said holes being aligned with each other.

31. An air bag as defined in claim 20 wherein:
said nipple comprises:
a shaft portion, said shaft portion having a through hole formed through the center thereof, said through hole having a female thread formed therein for allowing a male connector to be hermetically coupled thereto, while allowing air to pass through a central hole in said connector through said nipple into said air bag,

32. An air bag as defined in claim 20 wherein:
said nipple comprises:
a shaft portion;
a base portion formed along one end of said shaft portion; and
a head portion formed along the other end of said shaft portion,
said shaft portion, base portion and head portion having a through hole formed through the center thereof for allowing air to pass therethrough,
said base portion having a smooth upper surface so that it may be hermetically connected to the inside surface of said outer film,
said head portion being cylindrical in shape and having an outer diameter bigger than the outer diameter of said shaft portion, so that a clip can be hermetically mounted on said head portion.

33. An air bag as defined in claim 20 wherein:
said nipple is mounted in said outer film using conventional heat sealing techniques

34. An air bag as defined in claim 20 further comprising:
a double sided tape portion having a through hole formed in the center thereof, said double sided tape portion hermetically attaching said nipple to said outer film with said holes being aligned with each other.

35. An air bag as defined in claim 32 wherein said nipple further comprises:
means for preventing water from entering said nipple when said air bag is not being used to measure blood pressure.

36. An air bag as defined in claim 35, wherein:
said water preventing means comprises:
a flap integrally formed with said head portion along a central portion thereof; and
a round protrusion integrally formed with said flap portion on a central outwardly facing portion of said flap portion,
said flap portion and said round protrusion being cut through the centers thereof for allowing said flap and said round protrusion to deform and create an air passage therethrough when a clip is mounted on said nipple, thereby allowing air to pass through said nipple,
said nipple being formed of a resilient material.

37. An air bag as defined in claim 35, wherein said water preventing means comprises:
a flap integrally formed with said inside said shaft portion along a central portion thereof;
said flap potion being cut through the center thereof for allowing said to deform and create an air passage therethrough when a clip is mounted on said nipple, thereby allowing air to pass through said nipple,
said nipple being formed of a resilient material.

38. An air bag as defined in claim 32, wherein, said nipple further comprises:
uni-directional mounting means for allowing a clip to be mounted thereon in only one direction, so that a bar code scanner or RFR mounted in said clip can be properly aligned with a bar code or RFID mounted in said air bag, so that the identity of the patient on which said air bag is mounted on can be transmitted to said clip and to an electronic blood pressure measuring device coupled to said clip.

39. An air bag as defined in claim 38, wherein, said uni-directional mounting means comprises:
a blocking wall integrally formed with said shaft portion along one side thereof, said wall being larger than a slot in said clip.

40. An air bag for measuring blood pressure, which comprises:
a first film, said first film having a hole formed through a central portion thereof, said first film being long enough to go around a persons wrist;
a second film, having a hole formed through a central portion thereof, said second film being long enough to traverse the distance between the radius and the digital tendon of a persons' wrist:
a diaphragm mounted in said central hole in said first film, said diaphragm being long enough to traverse the distance between the radius and the digital tendon of a persons' wrist; and
a nipple mounted in said second film, said nipple having a through hole formed therein for allowing air to pass therethrough, said hole in said nipple and said hole in said second film being aligned with each other,
said first and second films being heat sealed to each other along the periphery of said diaphragm, said films being formed of a bendable and not stretchable material.

41. An air bag for measuring blood pressure, which comprises:
a first film, said first film having three holes formed through a central portion thereof, said first film being long enough to go around a persons' wrist;
a second film, having three holes formed through a central portion thereof, said second film being long enough to traverse distance between the radius and the digital tendon of a persons' wrist:
three diaphragms, each of which is mounted in a respective hole in said first film said diaphragms being long enough to traverse the distance between the radius and the digital tendon of a persons' wrist; and
three nipples each of which is mounted on said second film, the hole in each of said nipples being aligned with a respective hole in said second film;
said first and second films being heat sealed to each other along the periphery of each of said three diaphragms, thereby forming three air bags, each air bag having one of said diaphragms and one of said nipples on an upper and lower surface thereof, said films being formed of a bendable and not stretchable material.

42. An air bag as defined in claim 40, further comprising:
patient identification means for identifying the patient on which said air bag is mounted on: and
means for storing said patient identification means.

43. An air bag as defined in claim 42 wherein:
said storing means comprises a narrow pocket formed in said air bag, and
said identification means comprises a strip of paper having the name of the patient printed thereon.

44. An air bag as defined in claim 43 wherein:
said identification means further comprises an RFID device or bar code inserted in said pocket formed in said air bag for electronically identifying the patient on which said air bag is mounted on.

45. An air bag as defined in claim 40 wherein, said nipple comprises:
a shaft portion;
a head portion integrally formed with said shaft portion along one end of said shaft portion: and
a base portion integrally formed with said shaft portion along the other end of said shaft portion,
said shaft portion, head portion and base portion having a through hole formed therethrough for allowing air to pass through said nipple,
said base portion being hermetically connected to said second film.

46. A method of forming an air bag comprising the steps of:
a) cutting a first film, said first film being long enough to go around a persons hand, and having a width of about 40 mm.;
b) punching a round hole in a central portion of said first film;
c) cutting a second film, said second film being long enough to go around a persons hand, and having a width of about 40 mm.;
d) punching a round hole in a central portion of said second film;
e) attaching a double sided tape having a hole punched out of the center thereof to said first film the holes in said double sided tape and said first film being aligned with each other;
f) mounting a diaphragm on said double sided tape, so that the wave portion and the central portion of said diaphragm is outwardly exposed of said hole in said first film;
g) mounting a nipple, having a through hole through the center thereof, on said second film, with said through hole in said nipple and said hole in said second film being aligned with each other;
g) heat sealing said nipple to said second film; and
h) heat sealing said first and second films to each other in a pattern so that when air is pumped through said nipple, said diaphragm is caused to inflate.

47. A connector for connecting an air hose to an air bag for measuring blood pressure which comprises:
a cylindrical shaft portion, one end of said shaft having an air hose mounted thereon and the other end of said shaft having a male thread formed thereon, so that said male thread portion can be screwed into a female thread portion formed in a nipple mounted in an air bag, so that air being pumped from a pump in a electronic blood pressure measuring device can pass through said connector and through said nipple into said air bag.

48. A clip for connectively disconnecting an air bag from one end of an air hose comprising:
an upper rectangular arm portion;
a lower rectangular arm portion; and
biasing means for pressing the front ends of said upper and lower arms towards each other and the back ends of said arms away from each other;
air passage means for allowing air in said air hose to pass through said upper arm portion and into said air bag, so that air pumped into said air hose can pass through said upper arm into said air bag; and
air hose attaching means for attaching an air hose to said upper arm.

49. A clip as defined in claim 48 wherein, said biasing means comprises:
a rectangular bar portion, the respective ends of the bar portion being integrally formed with said arm portions along central portions of said arm portions.

50. A clip as defined in claim 48 wherein;
said lower arm portion has a slot formed therein, said slot extending from a front end of said lower arm portion, the width of said slot being the same as the outer diameter of a shaft portion of a nipple, and the length of said slot being substantially the same as the size of the outer diameter of a head portion of said nipple, whereby said shaft portion of said nipple can slide into said slot in said lower arm portion and said head portion of said nipple can be clamped between said upper and lower arm portions to form a hermetic seal therebetween, while allowing air to flow through said air passage means and through hole in said nipple.

51. A clip as defined in claim 48 wherein:
said biasing means comprises a spring.

52. A clip as defined in claim 48 wherein:
said biasing means comprises a spring sheet formed in the shape of a clip.

53. A clip as defined in claim 48 wherein:
said upper arm is in the shape of a cartoon figure, thereby relaxing the patient rather than causing anxiety in the patient about to have their blood pressure measured, which would result in a higher blood pressure reading.

54. A clip as defined in claim 48, wherein said clip further comprises:
means for storing a RFR or a bar code reader.

55. A clip as defined in claim 48, wherein said clip further comprises:
means for hermetically storing a RFR or a bar code reader inside the front end of said upper arm, the electrical wires in said RFR or bar code reader being supported inside said air passage means and inside said air hose attached to said upper arm.

56. An electronic blood pressure measuring device which comprises:
means for determining the actual air bag pressure on the artery AABPOA as a function of air volume in the air bag ABAV; and
means for determining the systolic and diastolic blood pressures as a function of the AABPOA.

57. An electronic blood pressure measuring device as defined in claim 56, wherein said AABPOA determining means comprises:
means for measuring the volume of air in said air bag:
means for measuring the air pressure in said air bag MAP as a function of the volume of air in said air bag;
means for storing a table of air bag ABS stretching pressure characteristics representative of the air pressure in the air bag as a function of the volume of air in said air bag while said air bag is not applied to a persons arm; and
subtracting means for subtracting said values stored in said ABS table from respective MAP pressures as a function of respective quantities of air in said air bag.

58. An electronic blood pressure measuring device which comprises:
ABS storing means for storing the air bag stretching pressure as a function of the air volume in the air bag;
an AABPOA calculating means for calculating the actual air bag pressure on the artery as a function of the air volume in said air bag;
means for calculating the systolic and diastolic blood pressure as a function of said AABPOA; and
means for displaying the thus calculated systolic and diastolic blood pressures.

59. An electronic blood pressure measuring device as defined in claim 58, wherein, said AABPOA determining means comprises:
means for measuring the volume of air inside an air bag;
means for measuring the air pressure inside the air bag as a function of the air volume inside the air bag;
air bag stretching characteristics storing means for storing data representative of the air pressure required to blow up the air bag as a function of the volume of air in said air bag.
subtracting means for subtracting said AABPOA from said MAP as a function of air volume in said air bag; and
means for storing a conventional algorithm for determining the systolic and diastolic blood pressures based on said calculated AABPOA and the shape of the blood pressure pulse provided by a pressure sensor as a function of time.

60. An arm band for a blood measuring device which comprises:
a band made of a bendable but substantially not stretchable material, said band being long enough to traverse a persons' wrist, and being about 30 mm wide,
said band having a first cavity formed on an outer surface thereof for receiving an electronic blood pressure measuring device therein,
said band having a second cavity formed on an inner surface thereof for receiving a diaphragm therein, formed on an inner surface thereof,
said band having a through hole formed therein, said through hole communicating air flow between said first and second cavities,
said first cavity being formed in said band along a central portion thereof and said second cavity being formed at a position which is substantially above the radial artery when said first cavity is over the center of the top of the a persons' wrist,
whereby, when said electronic blood pressure measuring device mounted in said first cavity is activated, air being pumped out of said device flows through said through hole causing said diaphragm mounted in said second cavity to expand outwards of said band and press against the radial artery in the persons' wrist.

61. An arm band as defined in claim 60, wherein said band further comprises:
a square wall portion formed around said first cavity, said device frictionally fitting inside said square wall to form a hermetic seal therebetween.

62. An arm band as defined in claim 50, wherein said second cavity has a groove formed along the side walls thereof for frictionally supporting a lip portion of said diaphragm therein, thereby not only physically supporting said diaphragm inside said second cavity but also providing a hermetic seal therebetween.

63. An arm band as defined in claim 60, wherein said band is formed of plastic using conventional injection molding techniques.

64. An arm band as defined in claim 60, wherein said band is formed of latex using conventional injection molding techniques.

65. An arm band as defined in claim 60, wherein said band is formed of silicone using conventional injection molding techniques.

66. An arm band as defined in claim 60, wherein said band is formed of rubber using conventional injection molding techniques.

67. An arm band as defined in claim 60, wherein said band further comprises two round cavities formed on the inner surface of said oval second cavity, said round cavities being provided for frictionally mounting an LED and a photo detector therein.

68. An arm band as defined in claim 67, wherein electrical wires of said LED and said photo detector are supported inside said through hole in said band, so that electrical signals and power between the device in said first cavity and said LED and photo cell in said second cavity can be transmitted by said wires in said through hole, thereby providing a simple, cheap and user friendly band form measuring blood pressure.

69. An arm band as defined in claim 50, wherein said band further comprises:
a cylindrical portion integrally formed with said band along the outer surface thereof, the hole in said cylindrical portion extending to said through hole in said band so that air can flow therebetween;
an manual air pump mounted on said cylindrical portion, so that when air pump is manually activated, air from said air pump passes through said hole in said cylindrical portion and said through hole in said pump causing said diaphragm mounted in said second cavity to expand outwards.
said first and second holes being in air communication with each other so that air pass therethrough

70. An arm band as defined in claim 60, wherein said band further comprises:
an oval ring, said ring being formed of a material which is not flexible; and
a diaphragm said diaphragm having an oval central portion and a oval lip portion integrally formed with said central portion, the outer diameter of said lip portion being smaller than said oval ring, so that when said lip portion is pulled over said ring, said central portion of said diaphragm is pre-stretched, thereby providing a linear diaphragm stretching characteristics,
said oval second cavity having an oval groove formed along an inner wall thereof, the shape and size of said oval groove being the same as the outer surface of said ring, so that said ring, having said diaphragm mounted thereon, can be physically pressed into said oval groove in said second cavity, thereby hermetically locking said pre-stretched diaphragm in said second cavity of said band.
